(19) United States
(12) Patent Application Publication
(10) Pub. No.: US 2026/0217514 A1
(43) Pub. Date: Jul. 30, 2026

(54) SURGICAL VIDEO ANALYSIS FOR OPERATIVE REPORTS VALIDATION

(71) Applicant: Theator Inc., Palo Alto, CA (US)

(72) Inventors: Tamir WOLF, Miami, FL (US); Dotan ASSELMANN, Holon (IL)

(73) Assignee: Theator Inc., Palo Alto, CA (US)

(21) Appl. No.: 19/574,101

(22) Filed: Mar. 20, 2026

Related U.S. Application Data
(63) Continuation-in-part of application No. 19/210,132, filed on May 16, 2025.
Application No. 19/210,132 is a continuation of application No. 18/354,763, filed on Jul. 19, 2023, now Pat. No. 12,334,200.
Application No. 18/354,763 is a continuation of application No. 17/829,108, filed on May 31, 2022, now Pat. No. 11,763,923.
Application No. 17/829,108 is a continuation of application No. 16/983,379, filed on Aug. 3, 2020, now Pat. No. 11,380,431.
Application No. 16/983,379 is a continuation of application No. 16/796,304, filed on Feb. 20, 2020, now Pat. No. 10,729,502.
(63) Continuation of application No. PCT/US2025/019807, filed on Mar. 13, 2025.
(60) Provisional application No. 62/808,512, filed on Feb. 21, 2019.
(60) Provisional application No. 62/808,500, filed on Feb. 21, 2019.
(60) Provisional application No. 62/838,066, filed on Apr. 24, 2019.
(60) Provisional application No. 62/960,466, filed on Jan. 13, 2020.
(60) Provisional application No. 62/967,283, filed on Jan. 29, 2020.
(60) Provisional application No. 63/571,304, filed on Mar. 28, 2024.
(60) Provisional application No. 63/716,091, filed on Nov. 4, 2024.

(51) Int. Cl. B66F 9/18; A61B 1/00; A61B 1/04; A61B 5/00; A61B 5/02; A61B 5/103; A61B 17/00; A61B 34/00; A61B 34/10; A61B 34/30; A61B 34/37; A61B 90/00; A61B 90/92; B25J 9/16; B65G 1/08; G06F 16/71; G06F 16/735; G06N 20/00; G06Q 10/0631; G06Q 40/08; G06T 7/00; G06V 20/20; G06V 20/40; G06V 20/52; G06V 40/10; G11B 27/10; G16H 10/40; G16H 10/60; G16H 15/00; G16H 20/40; G16H 30/20; G16H 30/40; G16H 40/20; G16H 40/63; G16H 50/20; G16H 50/30; G16H 50/70; G16H 70/20

(52) U.S. Cl. CPC B66F 9/183; A61B 1/000094; A61B 1/000096; A61B 5/02042; A61B 5/1032; A61B 5/7267; A61B 5/7275; A61B 5/746; A61B 17/00; A61B 34/10; A61B 34/25; A61B 34/30; A61B 34/37; A61B 34/70; A61B 90/06; A61B 90/37; A61B 90/92; B25J 9/1661; B65G 1/08; G06F 16/71; G06F 16/735; G06N 20/00; G06Q 10/063112; G06Q 10/06312; G06Q 40/08; G06T 7/0012; G06V 20/20; G06V 20/40; G06V 20/41; G06V 20/49; G06V 20/52; G11B 27/10; G16H 10/40; G16H 10/60; G16H 15/00; G16H 20/40; G16H 30/20; G16H 30/40; G16H 40/20; G16H 40/63; G16H 50/20; G16H 50/30; G16H 50/70; G16H 70/20; A61B 1/04; A61B 2017/0011; A61B 2017/00119; A61B 2034/104; A61B 2034/252; A61B 2034/254; A61B 2034/256; A61B 2034/302; A61B 2090/065; A61B 2090/0807; A61B 90/361; A61B 2090/364; A61B 2505/05; B65G 2201/0258; G06T 2207/10016; G06T 2207/30004; G06V 20/44; G06V 40/10; G06V 2201/034

(57) ABSTRACT

Systems, methods and non-transitory computer readable media for analysis of videos of surgical procedures are provided. One example includes surgical video analysis to inspect operative reports. Another example includes surgical video analysis for operative report text generation. Yet another example includes surgical video analysis for synchronized browsing of surgical videos and operative reports. An additional example includes surgical video analysis for resolving inconsistencies between surgical footages and operative reports.

### CROSS-REFERENCE TO RELATED APPLICATIONS

This application claims the benefit of priority of U.S. Provisional Patent Application No. 63/571,304 , filed on Mar. 28, 2024, and U.S. Provisional Patent Application No. 63/716,091 , filed on Nov. 4, 2024. The disclosures of which are incorporated herein by reference in their entirety.

### BACKGROUND OF THE INVENTION

### Technological Field

The disclosed embodiments generally relate to systems and methods for analysis of videos of surgical procedures.

### Background Information

Correct performance of surgical procedures, including the performance of the different steps of a surgical procedure in the right time and order, depends on the surgeon performing the surgical procedure. While surgeons are highly skilled and trained to avoid errors, errors do occur, as in any other human activity. While the training reduces the number of errors, the errors that do occur in a surgical procedure may have dire consequences. Having a peer or a supervisor in the operating room while the surgical procedure is ongoing to warn before an action is about to take place at an unsuitable phase of the ongoing surgical procedure may reduce the number of errors. This is a common solution when training new surgeons. However, the time and effort required from the peers and supervisors to oversee all surgeries, even of senior surgeons, will be enormous. Therefore, it is beneficial to have an automated detection of prospective adverse actions in surgical procedures.

Typically, a surgery is a focused endeavor to achieve a desired predetermined goals. However, during surgery, opportunities to perform other unplanned actions that may benefit the patient may arise. For example, an opportunity to treat a previously unknown condition that was discovered during the surgery may arise. In another example, an opportunity to diagnose a previously unsuspected condition may arise, for example through biopsy. Unfortunately, in many cases the surgeon conducting the surgery are focused on the desired predetermined goals, and may miss the opportunities to perform other unplanned actions that may benefit the patient. It is therefore beneficial to automatically identify the opportunities to perform other unplanned actions that may benefit the patient, and to notify the surgeons about the identified opportunities.

In a surgical procedure, a surgeon may be faced with many situations that require attention simultaneously. Some of these situations may be time sensitive, where a delayed reaction may be harmful. However, notifying the surgeon about all situations that require attention, or about all time sensitive situations, may result in clutter. It is therefore desired to identify the time sensitive situations that the surgeon is likely to miss, and notify the surgeon about these situations, possibly ignoring other situations or notifying about the other situations in a different, less intensive, way.

Therefore, there is a need for unconventional approaches that efficiently and effectively analyze surgical videos to enable a medical professional to receive support during and after ongoing surgical procedures.

In modern surgical practices, operative reports serve as critical documentation of procedures performed by healthcare providers. These reports are typically generated based on the surgeon's recollection and manual note-taking, which can lead to discrepancies between the documented report and the actual surgical footage. Such inconsistencies may result in inaccuracies in patient records, potential medico-legal issues, and challenges in quality assurance. While surgical video recording is becoming increasingly common, there is a lack of automated systems to cross-reference these recordings with corresponding operative reports. Accordingly, there is a need for improved systems and methods that can analyze surgical footage to identify inconsistencies in operative reports, ensuring greater accuracy, reliability, and accountability in surgical documentation.

Accurate and comprehensive documentation of surgical procedures is essential for patient care, legal compliance, and medical research. Traditionally, operative reports are manually created by healthcare providers, often after the procedure has been completed. This manual process can be time-consuming and prone to errors or omissions due to reliance on memory and subjective interpretation. With the increasing availability of surgical video recordings, there is an opportunity to automate the generation of operative reports by extracting relevant information directly from the recorded footage. However, existing solutions lack the ability to efficiently analyze surgical video data and convert it into structured text for documentation. Therefore, there is a need for systems and methods that utilize surgical video analysis to automatically generate accurate and consistent operative reports, reducing the burden on healthcare providers while improving the quality and completeness of surgical documentation.

Reviewing surgical procedures through both operative reports and recorded footage is essential for quality control, education, and legal verification. However, manually correlating textual reports with corresponding video segments is a time-consuming and inefficient process. Traditional methods require healthcare providers to independently search through surgical footage to locate specific moments referenced in reports, leading to inconsistencies and increased cognitive burden. Despite advancements in surgical video recording, there is a lack of integrated solutions that allow seamless synchronization between video content and textual documentation. Therefore, there is a need for systems and methods that enable synchronized browsing of surgical videos and operative reports, allowing users to efficiently navigate between textual descriptions and corresponding video segments. Such a system would enhance surgical documentation, facilitate review processes, and improve accessibility for training and audit purposes.

Ensuring the accuracy of operative reports is critical for patient safety, legal documentation, and medical research. However, discrepancies often arise between recorded surgical footage and preliminary operative reports due to human error, memory limitations, or time constraints in manual documentation. Identifying and resolving these inconsistencies is typically a labor-intensive process that requires manual review of both the video footage and the report, leading to inefficiencies and potential oversight. Existing systems lack automated tools to assist in detecting and resolving such inconsistencies efficiently. Therefore, there is a need for systems and methods that can analyze surgical footage, identify discrepancies in operative reports, and provide assisted resolution through suggested modifications. Such a solution would enhance the accuracy, consistency, and reliability of surgical documentation while reducing the burden on healthcare providers.

### SUMMARY OF THE INVENTION

In some examples, systems, methods and non-transitory computer readable media for surgical video analysis to inspect operative reports are provided. In one example, inconsistencies between the preliminary operative reports and the surgical footages may be identified. For example, an input of surgical footage of a surgical procedure performed on a patient by a health care provider may be received. Further, a preliminary operative report associated with the surgical procedure may be received. The preliminary operative report and/or the surgical footage may be analyzed to identify at least one inconsistency between the preliminary operative report and the surgical footage. An indication of the identified at least one inconsistency may be provided.

In some examples, systems, methods and non-transitory computer readable media for operative report text generation via surgical video analysis are provided. In one example, image-based text may be derived, for example by analyzing surgical footage of a surgical procedure for populating an operative report of the surgical procedure. For example, an input of an identifier of a patient may be received. Further, an input of an identifier of a health care provider may be received. Further, an input of surgical footage of a surgical procedure performed on the patient by the health care provider may be received. A plurality of frames of the surgical footage may be analyzed to derive image-based text for populating an operative report of the surgical procedure. The derived image-based text may be caused to populate the operative report of the surgical procedure.

In some examples, systems, methods and non-transitory computer readable media for synchronized browsing of surgical videos and operative reports are provided. For example, an input of surgical footage of a surgical procedure performed on a patient by a health care provider may be received. Further, textual data in a natural language may be received. The textual data may be associated with the surgical procedure. Further, a data-structure associating different portions of the surgical footage with different portions of the textual data may be accessed. Further, the textual data and a first part of the surgical footage may be presented to an individual, with a visual indication of a first part of the textual data. The first part of the surgical footage may be associated in the data-structure with the first part of the textual data. Further, a selection of a second part of the textual data may be received from the individual. Further, the data-structure may be used to determine a second part of the surgical footage associated with the second part of the textual data. Further, the second part of the surgical footage may be presented to the individual. Further, a selection of a third part of the surgical footage may be received from the individual. Further, the data-structure may be used to determine a third part of the textual data associated with the third part of the surgical footage. Further, a visual indication of the third part of the textual data may be presented to the individual.

In some examples, systems, methods and non-transitory computer readable media for assisted resolution of inconsistencies between surgical footages and operative reports are provided. For example, an input of surgical footage of a surgical procedure performed on a patient by a health care provider may be received. Further, a preliminary operative report associated with the surgical procedure may be received. Further, a data-structure indicative of a plurality of inconsistencies between the surgical footage and the preliminary operative report may be accessed. For each inconsistency of the plurality of inconsistencies, a respective visual indication of the inconsistency may be presented to an individual. The preliminary operative report may be presented to the individual. A selection of a particular inconsistency of the plurality of inconsistencies may be received from the individual. The preliminary operative report and/or the surgical footage may be analyzed to generate a suggested modification to the preliminary operative report to correct the particular inconsistency. The suggested modification may be presented to the individual. An input may be received from the individual indicative of a desire of the individual to alter the suggested modification. The preliminary operative report may be updated based on the suggested modification and the input received from the individual. The updated preliminary operative report may be presented to the individual.

### BRIEF DESCRIPTION OF DRAWINGS

FIG. 1 is a perspective view of an example operating room, consistent with disclosed embodiments.

FIG. 2 is a perspective view of cameras, consistent with disclosed embodiments.

FIG. 3 is a perspective view of an example of a surgical instrument, consistent with disclosed embodiments.

FIG. 4 illustrates an example timeline overlaid on a video of a surgical procedure consistent with the disclosed embodiments.

FIG. 5 is a flowchart illustrating an example process for reviewing surgical video, consistent with the disclosed embodiments.

FIG. 6 is a schematic illustration of an example data structure consistent with the disclosed embodiments.

FIG. 7 is an example operative report containing fields, consistent with disclosed embodiments.

FIG. 8A is an example of a process, including structure, for populating an operative report, consistent with disclosed embodiments.

FIG. 8B is another example of a process, including structure, for populating an operative report, consistent with disclosed embodiments.

FIG. 9 is a flow diagram of an exemplary process for populating an operative report, consistent with disclosed embodiments.

FIG. 10 is a flow diagram of an exemplary process, consistent with some embodiments of the present disclosure.

FIG. 11 is a flow diagram of an exemplary process, consistent with some embodiments of the present disclosure.

FIG. 12A is a flow diagram of an exemplary process for synchronized browsing of surgical videos and operative reports, consistent with some embodiments of the present disclosure.

FIG. 12B is a block diagram illustrating an example user interface, consistent with some embodiments of the present disclosure.

FIG. 13 is a flow diagram of an exemplary process for assisted resolution of inconsistencies between surgical footages and operative reports, consistent with some embodiments of the present disclosure.

### DETAILED DESCRIPTION OF THE INVENTION

Unless specifically stated otherwise, as apparent from the following description, throughout the specification discussions utilizing terms such as “processing”, “calculating”, “computing”, “determining”, “generating”, “setting”, “configuring”, “selecting”, “defining”, “applying”, “obtaining”, “monitoring”, “providing”, “identifying”, “segmenting”, “classifying”, “analyzing”, “associating”, “extracting”, “storing”, “receiving”, “transmitting”, or the like, include actions and/or processes of a computer that manipulate and/or transform data into other data, the data represented as physical quantities, for example such as electronic quantities, and/or the data representing physical objects. The terms “computer”, “processor”, “controller”, “processing unit”, “computing unit”, and “processing module” should be expansively construed to cover any kind of electronic device, component or unit with data processing capabilities, including, by way of non-limiting example, a personal computer, a wearable computer, smart glasses, a tablet, a smartphone, a server, a computing system, a cloud computing platform, a communication device, a processor (for example, digital signal processor (DSP), an image signal processor (ISR), a microcontroller, a field programmable gate array (FPGA), an application specific integrated circuit (ASIC), a central processing unit (CPA), a graphics processing unit (GPU), a visual processing unit (VPU), and so on), possibly with embedded memory, a single core processor, a multi core processor, a core within a processor, any other electronic computing device, or any combination of the above.

The operations in accordance with the teachings herein may be performed by a computer specially constructed or programmed to perform the described functions.

As used herein, the phrase “for example,” “such as”, “for instance” and variants thereof describe non-limiting embodiments of the presently disclosed subject matter. Reference in the specification to features of “embodiments” “one case”, “some cases”, “other cases” or variants thereof means that a particular feature, structure or characteristic described may be included in at least one embodiment of the presently disclosed subject matter. Thus, the appearance of such terms does not necessarily refer to the same embodiment(s). As used herein, the term “and/or” includes any and all combinations of one or more of the associated listed items.

Features of the presently disclosed subject matter, are, for brevity, described in the context of particular embodiments. However, it is to be understood that features described in connection with one embodiment are also applicable to other embodiments. Likewise, features described in the context of a specific combination may be considered separate embodiments, either alone or in a context other than the specific combination.

In embodiments of the presently disclosed subject matter, one or more stages illustrated in the figures may be executed in a different order and/or one or more groups of stages may be executed simultaneously and vice versa. The figures illustrate a general schematic of the system architecture in accordance embodiments of the presently disclosed subject matter. Each module in the figures can be made up of any combination of software, hardware and/or firmware that performs the functions as defined and explained herein. The modules in the figures may be centralized in one location or dispersed over more than one location.

Examples of the presently disclosed subject matter are not limited in application to the details of construction and the arrangement of the components set forth in the following description or illustrated in the drawings. The subject matter may be practiced or carried out in various ways. Also, it is to be understood that the phraseology and terminology employed herein is for the purpose of description and should not be regarded as limiting.

In this document, an element of a drawing that is not described within the scope of the drawing and is labeled with a numeral that has been described in a previous drawing may have the same use and description as in the previous drawings.

The drawings in this document may not be to any scale. Different figures may use different scales and different scales can be used even within the same drawing, for example different scales for different views of the same object or different scales for the two adjacent objects.

Consistent with disclosed embodiments, “at least one processor” may constitute any physical device or group of devices having electric circuitry that performs a logic operation on an input or inputs. For example, the at least one processor may include one or more integrated circuits (IC), including application-specific integrated circuit (ASIC), microchips, microcontrollers, microprocessors, all or part of a central processing unit (CPU), graphics processing unit (GPU), digital signal processor (DSP), field-programmable gate array (FPGA), server, virtual server, or other circuits suitable for executing instructions or performing logic operations. The instructions executed by at least one processor may, for example, be pre-loaded into a memory integrated with or embedded into the controller or may be stored in a separate memory. The memory may include a Random Access Memory (RAM), a Read-Only Memory (ROM), a hard disk, an optical disk, a magnetic medium, a flash memory, other permanent, fixed, or volatile memory, or any other mechanism capable of storing instructions. In some embodiments, the at least one processor may include more than one processor. Each processor may have a similar construction or the processors may be of differing constructions that are electrically connected or disconnected from each other. For example, the processors may be separate circuits or integrated in a single circuit. When more than one processor is used, the processors may be configured to operate independently or collaboratively. The processors may be coupled electrically, magnetically, optically, acoustically, mechanically or by other means that permit them to interact.

Disclosed embodiments may include and/or access a data structure. A data structure consistent with the present disclosure may include any collection of data values and relationships among them. The data may be stored linearly, horizontally, hierarchically, relationally, non-relationally, uni-dimensionally, multidimensionally, operationally, in an ordered manner, in an unordered manner, in an object-oriented manner, in a centralized manner, in a decentralized manner, in a distributed manner, in a custom manner, or in any manner enabling data access. By way of non-limiting examples, data structures may include an array, an associative array, a linked list, a binary tree, a balanced tree, a heap, a stack, a queue, a set, a hash table, a record, a tagged union, ER model, and a graph. For example, a data structure may include an XML database, an RDBMS database, an SQL database or NoSQL alternatives for data storage/search such as, for example, MongoDB, Redis, Couchbase, Datastax Enterprise Graph, Elastic Search, Splunk, SoIr, Cassandra, Amazon DynamoDB, Scylla, HBase, and Neo4J. A data structure may be a component of the disclosed system or a remote computing component (e.g., a cloud-based data structure). Data in the data structure may be stored in contiguous or non-contiguous memory. Moreover, a data structure, as used herein, does not require information to be co-located. It may be distributed across multiple servers, for example, that may be owned or operated by the same or different entities. Thus, the term “data structure” as used herein in the singular is inclusive of plural data structures.

In some embodiments, machine learning algorithms (also referred to as machine learning models in the present disclosure) may be trained using training examples, for example in the cases described below. Some non-limiting examples of such machine learning algorithms may include classification algorithms, data regressions algorithms, image segmentation algorithms, visual detection algorithms (such as object detectors, face detectors, person detectors, motion detectors, edge detectors, etc.), visual recognition algorithms (such as face recognition, person recognition, object recognition, etc.), speech recognition algorithms, mathematical embedding algorithms, natural language processing algorithms, support vector machines, random forests, nearest neighbors algorithms, deep learning algorithms, artificial neural network algorithms, convolutional neural network algorithms, recurrent neural network algorithms, linear machine learning models, non-linear machine learning models, ensemble algorithms, and so forth. For example, a trained machine learning algorithm may comprise an inference model, such as a predictive model, a classification model, a data regression model, a clustering model, a segmentation model, an artificial neural network (such as a deep neural network, a convolutional neural network, a recurrent neural network, etc.), a random forest, a support vector machine, and so forth. In some examples, the training examples may include example inputs together with the desired outputs corresponding to the example inputs. Further, in some examples, training machine learning algorithms using the training examples may generate a trained machine learning algorithm, and the trained machine learning algorithm may be used to estimate outputs for inputs not included in the training examples. In some examples, engineers, scientists, processes and machines that train machine learning algorithms may further use validation examples and/or test examples. For example, validation examples and/or test examples may include example inputs together with the desired outputs corresponding to the example inputs, a trained machine learning algorithm and/or an intermediately trained machine learning algorithm may be used to estimate outputs for the example inputs of the validation examples and/or test examples, the estimated outputs may be compared to the corresponding desired outputs, and the trained machine learning algorithm and/or the intermediately trained machine learning algorithm may be evaluated based on a result of the comparison. In some examples, a machine learning algorithm may have parameters and hyper parameters, where the hyper parameters may be set manually by a person or automatically by an process external to the machine learning algorithm (such as a hyper parameter search algorithm), and the parameters of the machine learning algorithm may be set by the machine learning algorithm based on the training examples. In some implementations, the hyper-parameters may be set based on the training examples and the validation examples, and the parameters may be set based on the training examples and the selected hyper-parameters. For example, given the hyper-parameters, the parameters may be conditionally independent of the validation examples.

In some embodiments, trained machine learning algorithms (also referred to as machine learning models and trained machine learning models in the present disclosure) may be used to analyze inputs and generate outputs, for example in the cases described below. In some examples, a trained machine learning algorithm may be used as an inference model that when provided with an input generates an inferred output. For example, a trained machine learning algorithm may include a classification algorithm, the input may include a sample, and the inferred output may include a classification of the sample (such as an inferred label, an inferred tag, and so forth). In another example, a trained machine learning algorithm may include a regression model, the input may include a sample, and the inferred output may include an inferred value corresponding to the sample. In yet another example, a trained machine learning algorithm may include a clustering model, the input may include a sample, and the inferred output may include an assignment of the sample to at least one cluster. In an additional example, a trained machine learning algorithm may include a classification algorithm, the input may include an image, and the inferred output may include a classification of an item depicted in the image. In yet another example, a trained machine learning algorithm may include a regression model, the input may include an image, and the inferred output may include an inferred value corresponding to an item depicted in the image (such as an estimated property of the item, such as size, volume, age of a person depicted in the image, cost of a product depicted in the image, and so forth). In an additional example, a trained machine learning algorithm may include an image segmentation model, the input may include an image, and the inferred output may include a segmentation of the image. In yet another example, a trained machine learning algorithm may include an object detector, the input may include an image, and the inferred output may include one or more detected objects in the image and/or one or more locations of objects within the image. In some examples, the trained machine learning algorithm may include one or more formulas and/or one or more functions and/or one or more rules and/or one or more procedures, the input may be used as input to the formulas and/or functions and/or rules and/or procedures, and the inferred output may be based on the outputs of the formulas and/or functions and/or rules and/or procedures (for example, selecting one of the outputs of the formulas and/or functions and/or rules and/or procedures, using a statistical measure of the outputs of the formulas and/or functions and/or rules and/or procedures, and so forth).

In some embodiments, artificial neural networks may be configured to analyze inputs and generate corresponding outputs. Some non-limiting examples of such artificial neural networks may comprise shallow artificial neural networks, deep artificial neural networks, feedback artificial neural networks, feed forward artificial neural networks, autoencoder artificial neural networks, probabilistic artificial neural networks, time delay artificial neural networks, convolutional artificial neural networks, recurrent artificial neural networks, long short term memory artificial neural networks, and so forth. In some examples, an artificial neural network may be configured manually. For example, a structure of the artificial neural network may be selected manually, a type of an artificial neuron of the artificial neural network may be selected manually, a parameter of the artificial neural network (such as a parameter of an artificial neuron of the artificial neural network) may be selected manually, and so forth. In some examples, an artificial neural network may be configured using a machine learning algorithm. For example, a user may select hyper-parameters for the an artificial neural network and/or the machine learning algorithm, and the machine learning algorithm may use the hyper-parameters and training examples to determine the parameters of the artificial neural network, for example using back propagation, using gradient descent, using stochastic gradient descent, using mini-batch gradient descent, and so forth. In some examples, an artificial neural network may be created from two or more other artificial neural networks by combining the two or more other artificial neural networks into a single artificial neural network.

In some embodiments, generative models may be configured to generate new content, such as textual content, visual content, auditory content, graphical content, and so forth. In some examples, generative models may generate new content without input. In other examples, generative models may generate new content based on an input. In one example, the new content may be fully determined from the input, where every usage of the generative model with the same input will produce the same new content. In another example, the new content may be associated with the input but not fully determined from the input, where every usage of the generative model with the same input may product a different new content that is associated with the input. In some examples, a generative model may be a result of training a machine learning generative algorithm with training examples. An example of such training example may include a sample input, together with a sample content associated with the sample input. Some non-limiting examples of such generative models may include Deep Generative Model (DGM), Generative Adversarial Network model (GAN), auto-regressive model, Variational AutoEncoder (VAE), transformers based generative model, artificial neural networks based generative model, hard-coded generative model, and so forth.

In some embodiments, generative models may be configured to generate new content, such as textual content, visual content, auditory content, graphical content, and so forth. In some examples, generative models may generate new content without input. In other examples, generative models may generate new content based on an input. In one example, the new content may be fully determined from the input, where every usage of the generative model with the same input will produce the same new content. In another example, the new content may be associated with the input but not fully determined from the input, where every usage of the generative model with the same input may product a different new content that is associated with the input. In some examples, a generative model may be a result of training a machine learning generative algorithm with training examples. An example of such training example may include a sample input, together with a sample content associated with the sample input. Some non-limiting examples of such generative models may include Deep Generative Model (DGM), Generative Adversarial Network model (GAN), auto-regressive model, Variational AutoEncoder (VAE), transformers based generative model, artificial neural networks based generative model, hard-coded generative model, and so forth.

A Large Language Model (LLM) is a generative language model with a large number of parameters (usually billions or more) trained on large corpus of unlabeled data (usually trillions of words or more) in a self-supervised learning scheme and/or a semi-supervised learning scheme. While models trained using a supervised learning scheme with label data are fitted to the specific tasks they were trained for, LLM can handle wide range of tasks that the model was never specifically trained for, including ill-defined tasks. It is common to provide LLM with instructions in natural language, sometimes referred to as prompts. For example, to cause a LLM to count the number of people that objected to a proposed plan in a meeting, one might use the following prompt, ‘Please read the meeting minutes. Of all the speakers in the meeting, please identify those who objected to the plan proposed by Mr. Smith at the beginning of the meeting. Please list their names, and count them.’ Further, after receiving a response from the LLM, it is common to refine the task or to provide subsequent tasks in natural language. For example, ‘Also count for each of these speakers the number of words said’, ‘Of these speakers, could you please identify who is the leader?’ or ‘Please summarize the main objections’. LLM may generate textual outputs in natural language, or in a desired structured format, such as a table or a formal language (such as a programming language, a digital file format, and so forth). In many cases, a LLM may be part of a multi-modal model and/or a foundation model, also referred to as multi-modal LLM or a multi-modal foundation model, allowing the model to analyze both textual inputs as well as other kind of inputs (such as images, videos, audio, sensor data, telemetries, and so forth) and/or to generate textual outputs as well as other kinds of outputs (such as images, videos, audio, telemetries, and so forth).

Some non-limiting examples of audio data may include audio recordings, audio stream, audio data that includes speech, audio data that includes music, audio data that includes ambient noise, digital audio data, analog audio data, digital audio signals, analog audio signals, mono audio data, stereo audio data, surround audio data, audio data captured using at least one audio sensor, audio data generated artificially, and so forth. In one example, audio data may be generated artificially from textual content, for example using text-to-speech algorithms. In another example, audio data may be generated using a generative machine learning model. In some embodiments, analyzing audio data (for example, by the methods, steps and modules described herein) may comprise analyzing the audio data to obtain a preprocessed audio data, and subsequently analyzing the audio data and/or the preprocessed audio data to obtain the desired outcome. One of ordinary skill in the art will recognize that the followings are examples, and that the audio data may be preprocessed using other kinds of preprocessing methods. In some examples, the audio data may be preprocessed by transforming the audio data using a transformation function to obtain a transformed audio data, and the preprocessed audio data may comprise the transformed audio data. For example, the transformation function may comprise a multiplication of a vectored time series representation of the audio data with a transformation matrix. For example, the transformation function may comprise convolutions, audio filters (such as low-pass filters, high-pass filters, band-pass filters, all-pass filters, etc.), linear functions, nonlinear functions, and so forth. In some examples, the audio data may be preprocessed by smoothing the audio data, for example using Gaussian convolution, using a median filter, and so forth. In some examples, the audio data may be preprocessed to obtain a different representation of the audio data. For example, the preprocessed audio data may comprise: a representation of at least part of the audio data in a frequency domain; a Discrete Fourier Transform of at least part of the audio data; a Discrete Wavelet Transform of at least part of the audio data; a time/frequency representation of at least part of the audio data; a spectrogram of at least part of the audio data; a log spectrogram of at least part of the audio data; a Mel-Frequency Spectrum of at least part of the audio data; a sonogram of at least part of the audio data; a periodogram of at least part of the audio data; a representation of at least part of the audio data in a lower dimension; a lossy representation of at least part of the audio data; a lossless representation of at least part of the audio data; a time order series of any of the above; any combination of the above; and so forth. In some examples, the audio data may be preprocessed to extract audio features from the audio data. Some non-limiting examples of such audio features may include: auto-correlation; number of zero crossings of the audio signal; number of zero crossings of the audio signal centroid; MP3 based features; rhythm patterns; rhythm histograms; spectral features, such as spectral centroid, spectral spread, spectral skewness, spectral kurtosis, spectral slope, spectral decrease, spectral roll-off, spectral variation, etc.; harmonic features, such as fundamental frequency, noisiness, inharmonicity, harmonic spectral deviation, harmonic spectral variation, tristimulus, etc.; statistical spectrum descriptors; wavelet features; higher level features; perceptual features, such as total loudness, specific loudness, relative specific loudness, sharpness, spread, etc.; energy features, such as total energy, harmonic part energy, noise part energy, etc.; temporal features; and so forth. In some examples, analyzing the audio data may include calculating at least one convolution of at least a portion of the audio data, and using the calculated at least one convolution to calculate at least one resulting value and/or to make determinations, identifications, recognitions, classifications, and so forth.

In some embodiments, analyzing audio data (for example, by the methods, steps and modules described herein) may comprise analyzing the audio data and/or the preprocessed audio data using one or more rules, functions, procedures, artificial neural networks, speech recognition algorithms, speaker recognition algorithms, speaker diarization algorithms, audio segmentation algorithms, noise cancelling algorithms, source separation algorithms, inference models, and so forth. Some non-limiting examples of such inference models may include: an inference model preprogrammed manually; a classification model; a data regression model; a result of training algorithms, such as machine learning algorithms and/or deep learning algorithms, on training examples, where the training examples may include examples of data instances, and in some cases, a data instance may be labeled with a corresponding desired label and/or result; and so forth.

Some non-limiting examples of image data may include one or more images, grayscale images, color images, series of images, 2D images, 3D images, videos, 2D videos, 3D videos, frames, footages, surgical footages, frames of surgical footage, surgical images, surgical videos, or data derived from other image data. In some embodiments, analyzing image data (for example by the methods, steps and modules described herein) may comprise analyzing the image data to obtain a preprocessed image data, and subsequently analyzing the image data and/or the preprocessed image data to obtain the desired outcome. One of ordinary skill in the art will recognize that the followings are examples, and that the image data may be preprocessed using other kinds of preprocessing methods. In some examples, the image data may be preprocessed by transforming the image data using a transformation function to obtain a transformed image data, and the preprocessed image data may comprise the transformed image data. For example, the transformed image data may comprise one or more convolutions of the image data. For example, the transformation function may comprise one or more image filters, such as low-pass filters, high-pass filters, band-pass filters, all-pass filters, and so forth. In some examples, the transformation function may comprise a nonlinear function. In some examples, the image data may be preprocessed by smoothing at least parts of the image data, for example using Gaussian convolution, using a median filter, and so forth. In some examples, the image data may be preprocessed to obtain a different representation of the image data. For example, the preprocessed image data may comprise: a representation of at least part of the image data in a frequency domain; a Discrete Fourier Transform of at least part of the image data; a Discrete Wavelet Transform of at least part of the image data; a time/frequency representation of at least part of the image data; a representation of at least part of the image data in a lower dimension; a lossy representation of at least part of the image data; a lossless representation of at least part of the image data; a time ordered series of any of the above; any combination of the above; and so forth. In some examples, the image data may be preprocessed to extract edges, and the preprocessed image data may comprise information based on and/or related to the extracted edges. In some examples, the image data may be preprocessed to extract image features from the image data. Some non-limiting examples of such image features may comprise information based on and/or related to: edges; corners; blobs; ridges; Scale Invariant Feature Transform (SIFT) features; temporal features; and so forth. In some examples, analyzing the image data may include calculating at least one convolution of at least a portion of the image data, and using the calculated at least one convolution to calculate at least one resulting value and/or to make determinations, identifications, recognitions, classifications, and so forth.

In some embodiments, analyzing image data (for example by the methods, steps and modules described herein) may comprise analyzing the image data to obtain a preprocessed image data, and subsequently analyzing the image data and/or the preprocessed image data to obtain the desired outcome. Some non-limiting examples of such image data may include one or more images, videos, frames, footages, 2D image data, 3D image data, and so forth. One of ordinary skill in the art will recognize that the followings are examples, and that the image data may be preprocessed using other kinds of preprocessing methods. In some examples, the image data may be preprocessed by transforming the image data using a transformation function to obtain a transformed image data, and the preprocessed image data may comprise the transformed image data. For example, the transformed image data may comprise one or more convolutions of the image data. For example, the transformation function may comprise one or more image filters, such as low-pass filters, high-pass filters, band-pass filters, all-pass filters, and so forth. In some examples, the transformation function may comprise a nonlinear function. In some examples, the image data may be preprocessed by smoothing at least parts of the image data, for example using Gaussian convolution, using a median filter, and so forth. In some examples, the image data may be preprocessed to obtain a different representation of the image data. For example, the preprocessed image data may comprise: a representation of at least part of the image data in a frequency domain; a Discrete Fourier Transform of at least part of the image data; a Discrete Wavelet Transform of at least part of the image data; a time/frequency representation of at least part of the image data; a representation of at least part of the image data in a lower dimension; a lossy representation of at least part of the image data; a lossless representation of at least part of the image data; a time ordered series of any of the above; any combination of the above; and so forth. In some examples, the image data may be preprocessed to extract edges, and the preprocessed image data may comprise information based on and/or related to the extracted edges. In some examples, the image data may be preprocessed to extract image features from the image data. Some non-limiting examples of such image features may comprise information based on and/or related to edges; corners; blobs; ridges; Scale Invariant Feature Transform (SIFT) features; temporal features; and so forth.

In some embodiments, analyzing image data (for example, by the methods, steps and modules described herein) may comprise analyzing the image data and/or the preprocessed image data using one or more rules, functions, procedures, artificial neural networks, object detection algorithms, face detection algorithms, visual event detection algorithms, action detection algorithms, motion detection algorithms, background subtraction algorithms, inference models, and so forth. Some non-limiting examples of such inference models may include: an inference model preprogrammed manually; a classification model; a regression model; a result of training algorithms, such as machine learning algorithms and/or deep learning algorithms, on training examples, where the training examples may include examples of data instances, and in some cases, a data instance may be labeled with a corresponding desired label and/or result; and so forth.

In some embodiments, analyzing image data (for example, by the methods, steps and modules described herein) may comprise analyzing pixels, voxels, point cloud, range data, etc. included in the image data.

A convolution may include a convolution of any dimension. A one-dimensional convolution is a function that transforms an original sequence of numbers to a transformed sequence of numbers. The one-dimensional convolution may be defined by a sequence of scalars. Each particular value in the transformed sequence of numbers may be determined by calculating a linear combination of values in a subsequence of the original sequence of numbers corresponding to the particular value. A result value of a calculated convolution may include any value in the transformed sequence of numbers. Likewise, an n-dimensional convolution is a function that transforms an original n-dimensional array to a transformed array. The n-dimensional convolution may be defined by an n-dimensional array of scalars (known as the kernel of the n-dimensional convolution). Each particular value in the transformed array may be determined by calculating a linear combination of values in an n-dimensional region of the original array corresponding to the particular value. A result value of a calculated convolution may include any value in the transformed array. In some examples, an image may comprise one or more components (such as color components, depth component, etc.), and each component may include a two dimensional array of pixel values. In one example, calculating a convolution of an image may include calculating a two dimensional convolution on one or more components of the image. In another example, calculating a convolution of an image may include stacking arrays from different components to create a three dimensional array, and calculating a three dimensional convolution on the resulting three dimensional array. In some examples, a video may comprise one or more components (such as color components, depth component, etc.), and each component may include a three dimensional array of pixel values (with two spatial axes and one temporal axis). In one example, calculating a convolution of a video may include calculating a three dimensional convolution on one or more components of the video. In another example, calculating a convolution of a video may include stacking arrays from different components to create a four dimensional array, and calculating a four dimensional convolution on the resulting four dimensional array. In some examples, audio data may comprise one or more channels, and each channel may include a stream or a one-dimensional array of values. In one example, calculating a convolution of audio data may include calculating a one dimensional convolution on one or more channels of the audio data. In another example, calculating a convolution of audio data may include stacking arrays from different channels to create a two dimensional array, and calculating a two dimensional convolution on the resulting two dimensional array.

Some non-limiting examples of a mathematical object in a mathematical space may include a mathematical point in the mathematical space, a group of mathematical points in the mathematical space (such as a region, a manifold, a mathematical subspace, etc.), a mathematical shape in the mathematical space, a numerical value, a vector, a matrix, a tensor, a function, and so forth. Another non-limiting example of a mathematical object is a vector, wherein the dimension of the vector may be at least two (for example, exactly two, exactly three, more than three, and so forth). Some non-limiting examples of a phrase may include a phrase of at least two words, a phrase of at least three words, a phrase of at least five words, a phrase of more than ten words, and so forth. In some examples, a particular mathematical object may correspond to a particular word, may correspond to a particular utterance, may correspond to particular natural language data, and so forth. In one example, the particular word, the particular utterance, or the particular natural language data may be determined based on the particular mathematical object. For example, the injective function described below may be used to determine the particular word, the particular utterance, or the particular natural language data corresponding to the particular mathematical object.

In some embodiments, a mathematical object in a particular mathematical space may be determined. The mathematical object may correspond to and/or be determined based on natural language data (such as a word, a phrase, a sentence, a paragraph, an utterance, textual data, audible data including the natural language data, and so forth). In one example, the mathematical object may be determined based on the natural language data. For example, a function or an injective function mapping words and/or phrases and/or natural language inputs to mathematical objects in the particular mathematical space may be used based on the natural language data to obtain the mathematical object corresponding to the natural language data. For example, a word2vec or a Global Vectors for Word Representation (GloVe) algorithm may be used to obtain the function. In another example, a word or text embedding algorithm may be used to obtain the function.

In some embodiments, a mathematical object in a particular mathematical space may be identified based on particular information. For example, the particular information may be or include natural language data, and the mathematical object may be identified based on the natural language data as described above. In another example, the particular information may be or include the mathematical object, and the particular information may be accessed to obtain the mathematical object. In yet another example, the particular information may be or include a numerical value, and a function of the numerical value may be calculated to obtain the mathematical object. Some non-limiting examples of such function may include a linear function, a non-linear function, a polynomial function, an exponential function, a logarithmic function, a continuous function, a discontinuous function, and so forth. In some examples, a trained machine learning model may be used to analyze the particular information to determine the mathematical object. The trained machine learning model may be a machine learning model trained using training examples to determine mathematical objects based on information. An example of such training example may include sample information, together with a label indicative of a mathematical object.

In some examples, a function of two mathematical objects in a particular mathematical space to obtain a particular mathematical object in the particular mathematical space. In one example, a function of a plurality of mathematical objects (such as two mathematical objects, three mathematical objects, four mathematical objects, more than four mathematical objects, etc.) in a particular mathematical space may be calculated to obtain a particular mathematical object in the particular mathematical space. In one example, a function of at least one mathematical object (such as a single mathematical object, two mathematical objects, three mathematical objects, four mathematical objects, more than four mathematical objects, etc.) in a particular mathematical space and/or at least one numerical value (such as a single numerical value, two numerical values, three numerical values, four numerical values, more than four numerical values, etc.) may be calculated to obtain a particular mathematical object in the particular mathematical space. Some non-limiting examples of such function may include a linear function, a non-linear function, a polynomial function, an exponential function, a logarithmic function, a continuous function, a discontinuous function, and so forth. In one example, the particular mathematical object may correspond to a particular word, may correspond to a particular utterance, may correspond to particular natural language data, and so forth. In one example, the particular word, the particular utterance, or the particular natural language data may be determined based on the particular mathematical object. For example, the injective function described above may be used to determine the particular word, the particular utterance, or the particular natural language data corresponding to the particular mathematical object.

In some examples, a function of two or more pluralities of numerical values may be calculated to obtain a particular mathematical object in a particular mathematical space. The particular mathematical object may correspond to particular word, a particular utterance, or particular natural language data. Some non-limiting examples of such function may include a linear function, a non-linear function, a polynomial function, an exponential function, a logarithmic function, a continuous function, a discontinuous function, and so forth. In one example, the particular word, the particular utterance, or the particular natural language data may be determined based on the particular mathematical object. For example, the injective function described above may be used to determine the particular word corresponding to the particular mathematical object.

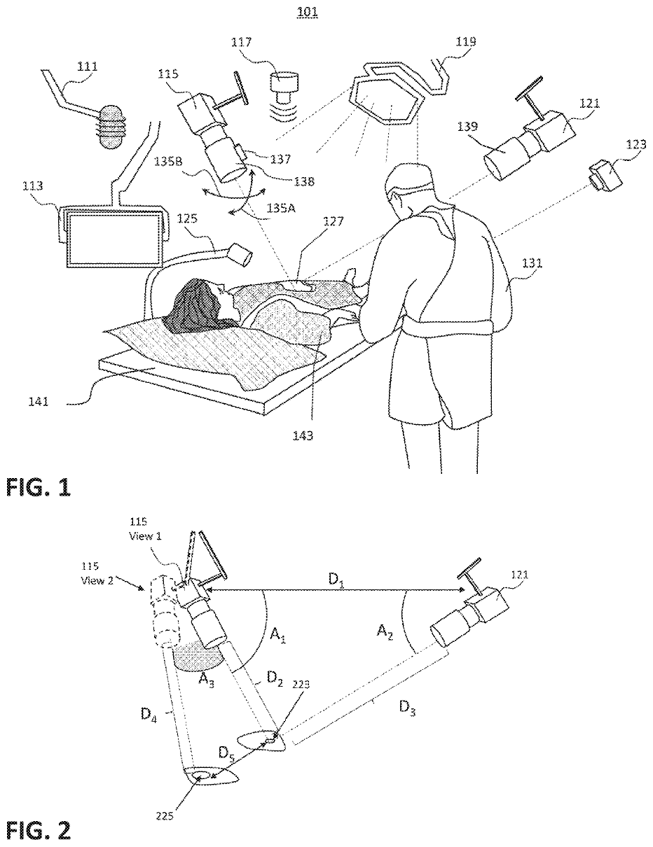

FIG. 1 shows an example operating room 101, consistent with disclosed embodiments. A patient 143 is illustrated on an operating table 141. Room 101 may include audio sensors, video/image sensors, chemical sensors, and other sensors, as well as various light sources (e.g., light source 119 is shown in FIG. 1) for facilitating the capture of video and audio data, as well as data from other sensors, during the surgical procedure. For example, room 101 may include one or more microphones (e.g., audio sensor 111, as shown in FIG. 1), several cameras (e.g., overhead cameras 115, 121, and 123, and a tableside camera 125) for capturing video/image data during surgery. While some of the cameras (e.g., cameras 115, 123 and 125) may capture video/image data of operating table 141 (e.g., the cameras may capture the video/image data at a location 127 of a body of patient 143 on which a surgical procedure is performed), camera 121 may capture video/image data of other parts of operating room 101. For instance, camera 121 may capture video/image data of a surgeon 131 performing the surgery. In some cases, cameras may capture video/image data associated with surgical team personnel, such as an anesthesiologist, nurses, surgical tech and the like located in operating room 101. Additionally, operating room cameras may capture video/image data associated with medical equipment located in the room.

In various embodiments, one or more of cameras 115, 121, 123 and 125 may be movable. For example, as shown in FIG. 1, camera 115 may be rotated as indicated by arrows 135A showing a pitch direction, and arrows 135B showing a yaw direction for camera 115. In various embodiments, pitch and yaw angles of cameras (e.g., camera 115) may be electronically controlled such that camera 115 points at a region-of-interest (ROI), of which video/image data needs to be captured. For example, camera 115 may be configured to track a surgical instrument (also referred to as a surgical tool) within location 127, an anatomical structure, a hand of surgeon 131, an incision, a movement of anatomical structure, and the like. In various embodiments, camera 115 may be equipped with a laser 137 (e.g., an infrared laser) for precision tracking. In some cases, camera 115 may be tracked automatically via a computer-based camera control application that uses an image recognition algorithm for positioning the camera to capture video/image data of a ROI. For example, the camera control application may identify an anatomical structure, identify a surgical tool, hand of a surgeon, bleeding, motion, and the like at a particular location within the anatomical structure, and track that location with camera 115 by rotating camera 115 by appropriate yaw and pitch angles. In some embodiments, the camera control application may control positions (i.e., yaw and pitch angles) of various cameras 115, 121, 123 and 125 to capture video/image date from different ROIs during a surgical procedure. Additionally or alternatively, a human operator may control the position of various cameras 115, 121, 123 and 125, and/or the human operator may supervise the camera control application in controlling the position of the cameras.

Cameras 115, 121, 123 and 125 may further include zoom lenses for focusing in on and magnifying one or more ROIs. In an example embodiment, camera 115 may include a zoom lens 138 for zooming closely to a ROI (e.g., a surgical tool in the proximity of an anatomical structure). Camera 121 may include a zoom lens 139 for capturing video/image data from a larger area around the ROI. For example, camera 121 may capture video/image data for the entire location 127. In some embodiments, video/image data obtained from camera 121 may be analyzed to identify a ROI during the surgical procedure, and the camera control application may be configured to cause camera 115 to zoom towards the ROI identified by camera 121.

In various embodiments, the camera control application may be configured to coordinate the position, focus, and magnification of various cameras during a surgical procedure. For example, the camera control application may direct camera 115 to track an anatomical structure and may direct camera 121 and 125 to track a surgical instrument. Cameras 121 and 125 may track the same ROI (e.g., a surgical instrument) from different view angles. For example, video/image data obtained from different view angles may be used to determine the position of the surgical instrument relative to a surface of the anatomical structure, to determine a condition of an anatomical structure, to determine pressure applied to an anatomical structure, or to determine any other information where multiple viewing angles may be beneficial. By way of another example, bleeding may be detected by one camera, and one or more other cameras may be used to identify the source of the bleeding.

In various embodiments, control of position, orientation, settings, and/or zoom of cameras 115, 121, 123 and 125 may be rule-based and follow an algorithm developed for a given surgical procedure. For example, the camera control application may be configured to direct camera 115 to track a surgical instrument, to direct camera 121 to location 127, to direct camera 123 to track the motion of the surgeon's hands, and to direct camera 125 to an anatomical structure. The algorithm may include any suitable logical statements determining position, orientation, settings and/or zoom for cameras 115, 121, 123 and 125 depending on various events during the surgical procedure. For example, the algorithm may direct at least one camera to a region of an anatomical structure that develops bleeding during the procedure. Some non-limiting examples of settings of cameras 115, 121, 123 and 125 that may be controlled (for example by the camera control application) may include image pixel resolution, frame rate, image and/or color correction and/or enhancement algorithms, zoom, position, orientation, aspect ratio, shutter speed, aperture, focus, and so forth.

In various cases, when a camera (e.g., camera 115) tracks a moving or deforming object (e.g., when camera 115 tracks a moving surgical instrument, or a moving/pulsating anatomical structure), a camera control application may determine a maximum allowable zoom for camera 115, such that the moving or deforming object does not escape a field of view of the camera. In an example embodiment, the camera control application may initially select the first zoom for camera 115, evaluate whether the moving or deforming object escapes the field of view of the camera, and adjust the zoom of the camera as necessary to prevent the moving or deforming object from escaping the field of view of the camera. In various embodiments, the camera zoom may be readjusted based on a direction and a speed of the moving or deforming object.

In various embodiments, one or more image sensors may include moving cameras 115, 121, 123 and 125. Cameras 115, 121, 123 and 125 may be used for determining sizes of anatomical structures and determining distances between different ROIs, for example using triangulation. For example, FIG. 2 shows exemplary cameras 115 (115 View 1, as shown in FIGS. 2) and 121 supported by movable elements such that the distance between the two cameras is D1, as shown in FIG. 2. Both cameras point at ROI 223. By knowing the positions of cameras 115 and 121 and the direction of an object relative to the cameras (e.g., by knowing angles A1 and A2, as shown in FIG. 2, for example based on correspondences between pixels depicting the same object or the same real-world point in the images captured by 115 and 121), distances D2 and D3 may be calculated using, for example, the law of sines and the known distance between the two cameras D1. In an example embodiment, when camera 115 (115, View 2) rotates by a small angle A3 (measured in radians), to point at ROI 225, the distance between ROI 223 and ROI 225 may be approximated (for small angles A3) by A3D2. More accuracy may be obtained using another triangulation process. Knowing distances between ROI 223 and 225 allows determining a length scale for an anatomical structure. Further, distances between various points of the anatomical structure, and distances from the various points to one or more cameras may be measured to determine a point-cloud representing a surface of the anatomical structure. Such a point-cloud may be used to reconstruct a three-dimensional model of the anatomical structure. Further, distances between one or more surgical instruments and different points of the anatomical structure may be measured to determine proper locations of the one or more surgical instruments in the proximity of the anatomical structure. In some other examples, one or more of cameras 115, 121, 123 and 125 may include a 3D camera (such as a stereo camera, an active stereo camera, a Time of Flight camera, a Light Detector and Ranging camera, etc.), and actual and/or relative locations and/or sizes of objects within operating room 101, and/or actual distances between objects, may be determined based on the 3D information captured by the 3D camera.

Returning to FIG. 1, light sources (e.g., light source 119) may also be movable to track one or more ROIs. In an example embodiment, light source 119 may be rotated by yaw and pitch angles, and in some cases, may extend towards to or away from a ROI (e.g., location 127). In some cases, light source 119 may include one or more optical elements (e.g., lenses, flat or curved mirrors, and the like) to focus light on the ROI. In some cases, light source 119 may be configured to control the color of the light (e.g., the color of the light may include different types of white light, a light with a selected spectrum, and the like). In an example embodiment, light 119 may be configured such that the spectrum and intensity of the light may vary over a surface of an anatomic structure illuminated by the light. For example, in some cases, light 119 may include infrared wavelengths which may result in warming of at least some portions of the surface of the anatomic structure.

In some embodiments, the operating room may include sensors embedded in various components depicted or not depicted in FIG. 1. Examples of such sensors may include: audio sensors; image sensors; motion sensors; positioning sensors; chemical sensors; temperature sensors; barometers; pressure sensors; proximity sensors; electrical impedance sensors; electrical voltage sensors; electrical current sensors; or any other detector capable of providing feedback on the environment or a surgical procedure, including, for example, any kind of medical or physiological sensor configured to monitor patient 143.

In some embodiments, audio sensor 111 may include one or more audio sensors configured to capture audio by converting sounds to digital information (e.g., audio sensors 121).

In various embodiments, temperature sensors may include infrared cameras (e.g., an infrared camera 117 is shown in FIG. 1) for thermal imaging. Infrared camera 117 may allow measurements of the surface temperature of an anatomic structure at different points of the structure. Similar to visible cameras D115, 121, 123 and 125, infrared camera 117 may be rotated using yaw or pitch angles. Additionally or alternatively, camera 117 may include an image sensor configured to capture image from any light spectrum, include infrared image sensor, hyper-spectral image sensors, and so forth.

FIG. 1 includes a display screen 113 that may show views from different cameras 115, 121, 123 and 125, as well as other information. For example, display screen 113 may show a zoomed-in image of a tip of a surgical instrument and a surrounding tissue of an anatomical structure in proximity to the surgical instrument.

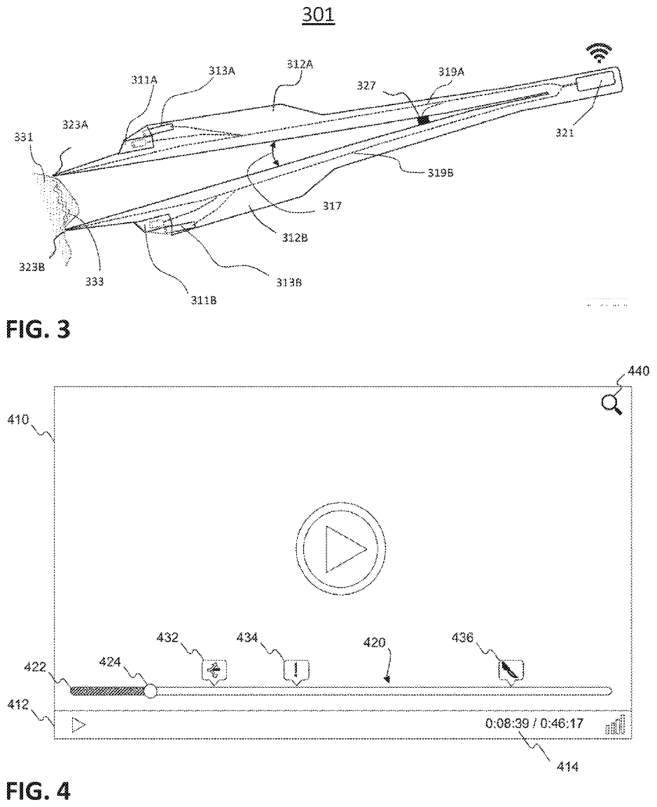

FIG. 3 shows an example embodiment of a surgical instrument 301 that may include multiple sensors and light-emitting sources. Consistent with the present embodiments, a surgical instrument may refer to a medical device, a medical instrument, an electrical or mechanical tool, a surgical tool, a diagnostic tool, and/or any other instrumentality that may be used during a surgery. As shown, instrument 301 may include cameras 311A and 311B, light sources 313A and 313B as well as tips 323A and 323B for contacting tissue 331. Cameras 311A and 311B may be connected via data connection 319A and 319B to a data transmitting device 321. In an example embodiment, device 321 may transmit data to a data-receiving device using a wireless communication or using a wired communication. In an example embodiment, device 321 may use WiFi, Bluetooth, NFC communication, inductive communication, or any other suitable wireless communication for transmitting data to a data-receiving device. The data-receiving device may include any form of receiver capable of receiving data transmissions. Additionally or alternatively, device 321 may use optical signals to transmit data to the data-receiving device (e.g., device 321 may use optical signals transmitted through the air or via optical fiber). In some embodiments, device 301 may include local memory for storing at least some of the data received from sensors 311A and 311B. Additionally, device 301 may include a processor for compressing video/image data before transmitting the data to the data-receiving device.

In various embodiments, for example when device 301 is wireless, it may include an internal power source (e.g., a battery, a rechargeable battery, and the like) and/or a port for recharging the battery, an indicator for indicating the amount of power remaining for the power source, and one or more input controls (e.g., buttons) for controlling the operation of device 301. In some embodiments, control of device 301 may be accomplished using an external device (e.g., a smartphone, tablet, smart glasses) communicating with device 301 via any suitable connection (e.g., WiFi, Bluetooth, and the like). In an example embodiment, input controls for device 301 may be used to control various parameters of sensors or light sources. For example, input controls may be used to dim/brighten light sources 313A and 313B, move the light sources for cases when the light sources may be moved (e.g., the light sources may be rotated using yaw and pitch angles), control the color of the light sources, control the focusing of the light sources, control the motion of cameras 311A and 311B for cases when the cameras may be moved (e.g., the cameras may be rotated using yaw and pitch angles), control the zoom and/or capturing parameters for cameras 311A and 311B, or change any other suitable parameters of cameras 311A-311B and light sources 313A-313B. It should be noted camera 311A may have a first set of parameters and camera 311B may have a second set of parameters that is different from the first set of parameters, and these parameters may be selected using appropriate input controls. Similarly, light source 313A may have a first set of parameters and light source 313B may have a second set of parameters that is different from the first set of parameters, and these parameters may be selected using appropriate input controls.

Additionally, instrument 301 may be configured to measure data related to various properties of tissue 331 via tips 323A and 323B and transmit the measured data to device 321. For example, tips 323A and 323B may be used to measure the electrical resistance and/or impedance of tissue 331, the temperature of tissue 331, mechanical properties of tissue 331 and the like. To determine elastic properties of tissue 331, for example, tips 323A and 323B may be first separated by an angle 317 and applied to tissue 331. The tips may be configured to move such as to reduce angle 317, and the motion of tips may result in pressure on tissue 331. Such pressure may be measured (e.g., via a piezoelectric element 327 that may be located between a first branch 312A and a second branch 312B of instrument 301), and based on the change in angle 317 (i.e., strain) and the measured pressure (i.e., stress), the elastic properties of tissue 331 may be measured. Furthermore, based on angle 317 distance between tips 323A and 323B may be measured, and this distance may be transmitted to device 321. Such distance measurements may be used as a length scale for various video/image data that may be captured by various cameras 115, 121, 123 and 125, as shown in FIG. 1.

Instrument 301 is only one example of possible surgical instrument, and other surgical instruments such as scalpels, graspers (e.g., forceps), clamps and occluders, needles, retractors, cutters, dilators, suction tips, and tubes, sealing devices, irrigation and injection needles, scopes and probes, and the like, may include any suitable sensors and light-emitting sources. In various cases, the type of sensors and light-emitting sources may depend on a type of surgical instrument used for a surgical procedure. In various cases, these other surgical instruments may include a device similar to device 301, as shown in FIG. 3, for collecting and transmitting data to any suitable data-receiving device.

When preparing for a surgical procedure, it may be beneficial for a surgeon to review video footage of surgical procedures having similar surgical events. It may be too time consuming, however, for a surgeon to view the entire video or to skip around to find relevant portions of the surgical footage. Therefore, there is a need for unconventional approaches that efficiently and effectively enable a surgeon to view a surgical video summary that aggregates footage of relevant surgical events while omitting other irrelevant footage.

Aspects of this disclosure may relate to reviewing surgical video, including methods, systems, devices, and computer readable media. An interface may allow a surgeon to review surgical video (of their own surgeries, other's surgeries, or compilations) with a surgical timeline simultaneously displayed. The timeline may include markers keyed to activities or events that occur during a surgical procedure. These markers may allow the surgeon to skip to particular activities to thereby streamline review of the surgical procedure. In some embodiments, key decision making junction points may be marked, and the surgeon may be permitted to view alternative actions taken at those decision making junction points.

For ease of discussion, a method is described below, with the understanding that aspects of the method apply equally to systems, devices, and computer readable media. For example, some aspects of such a method may occur electronically over a network that is either wired, wireless, or both. Other aspects of such a method may occur using non-electronic means. In a broadest sense, the method is not limited to particular physical and/or electronic instrumentalities, but rather may be accomplished using many differing instrumentalities.

Consistent with disclosed embodiments, a method may involve accessing at least one video of a surgical procedure. As described in greater detail above, video may include any form of recorded visual media including recorded images and/or sound. The video may be stored as a video file such as an Audio Video Interleave (AVI) file, a Flash Video Format (FLV) file, QuickTime File Format (MOV), MPEG (MPG, MP4, M4P, etc.), a Windows Media Video (WMV) file, a Material Exchange Format (MXF) file, or any other suitable video file formats, for example as described above.

A surgical procedure may include any medical procedure associated with or involving manual or operative procedures on a patient's body. Surgical procedures may include cutting, abrading, suturing, or other techniques that involve physically changing body tissues and organs. Examples of such surgical procedures are provided above. A video of a surgical procedure may include any series of still images that were captured during and are associated with the surgical procedure. In some embodiments, at least a portion of the surgical procedure may be depicted in one or more of the still images included in the video. For example, the video of the surgical procedure may be recorded by an image capture device, such as a camera, in an operating room or in a cavity of a patient. Accessing the video of the surgical procedure may include retrieving the video from a storage device (such as one or more memory units, a video server, a cloud storage platform, or any other storage platform), receiving the video from another device through a communication device, capturing the video using image sensors, or any other means for electronically accessing data or files.

Some aspects of the present disclosure may involve causing the at least one video to be output for display. Outputting the at least one video may include any process by which the video is produced, delivered, or supplied using a computer or at least one processor. As used herein, “display” may refer to any manner in which a video may be presented to a user for playback. In some embodiments, outputting the video may include presenting the video using a display device, such as a screen (e.g., an OLED, QLED LCD, plasma, CRT, DLPT, electronic paper, or similar display technology), a light projector (e.g., a movie projector, a slide projector), a 3D display, screen of a mobile device, electronic glasses or any other form of visual and/or audio presentation. In other embodiments, outputting the video for display may include storing the video in a location that is accessible by one or more other computing devices. Such storage locations may include a local storage (such as a hard drive of flash memory), a network location (such as a server or database), a cloud computing platform, or any other accessible storage location. The video may be accessed from a separate computing device for display on the separate computing device. In some embodiments, outputting the video may include transmitting the video to an external device. For example, outputting the video for display may include transmitting the video through a network to a user device for playback on the user device.

Embodiments of the present disclosure may further include overlaying on the at least one video outputted for display a surgical timeline. As used herein, a “timeline” may refer to any depiction from which a sequence of events may be tracked or demarcated. In some embodiments, a timeline may be a graphical representation of events, for example, using an elongated bar or line representing time with markers or other indicators of events along the bar. A timeline may also be a text-based list of events arranged in chronological order. A surgical timeline may be a timeline representing events associated with a surgery. As one example, a surgical timeline may be a timeline of events or actions that occur during a surgical procedure, as described in detail above. In some embodiments, the surgical timeline may include textual information identifying portions of the surgical procedure. For example, the surgical timeline may be a list of descriptions of intraoperative surgical events or surgical phases within a surgical procedure. In other embodiments, by hovering over or otherwise actuating graphical markers on a timeline, a descriptor associated with the marker may appear.

Overlaying the surgical timeline on the at least one video may include any manner of displaying the surgical timeline such that it can be viewed simultaneously with the at least one video. In some embodiments, overlaying the video may include displaying the surgical timeline such that it at least partially overlaps the video. For example, the surgical timeline may be presented as a horizontal bar along a top or bottom of the video or a vertical bar along a side of the video. In other embodiments, overlaying may include presenting the surgical timeline alongside the video. For example, the video may be presented on a display with the surgical timeline presented above, below, and/or to the side of the video. The surgical timeline may be overlaid on the video while the video is being played. Thus, “overlaying” as used herein refers more generally to simultaneous display. The simultaneous display may or may not be constant. For example, the overlay may appear with the video output before the end of the surgical procedure depicted in the displayed video. Or, the overlay may appear during substantially all of the video procedure.

FIG. 4 illustrates an example timeline 420 overlaid on a video of a surgical procedure consistent with the disclosed embodiments. The video may be presented in a video playback region 410, which may sequentially display one or more frames of the video. In the example shown in FIG. 4, timeline 420 may be displayed as a horizontal bar representing time, with the leftmost portion of the bar representing a beginning time of the video and the rightmost portion of the bar representing an end time. Timeline 420 may include a position indicator 424 indicating the current playback position of the video relative to the timeline. Colored region 422 of timeline 420 may represent the progress within timeline 420 (e.g., corresponding to video that has already been viewed by the user, or to video coming before the currently presented frame). In some embodiments, position indicator 424 may be interactive, such that the user can move to different positions within the video by moving position indicator 424. In some embodiments, the surgical timeline may include markers identifying at least one of a surgical phase, an intraoperative surgical event, and a decision making junction. For example, timeline 420 may further include one or more markers 432, 434, and/or 436. Such markers are described in greater detail below.

In the example shown in FIG. 4, timeline 420 may be displayed such that it overlaps video playback region 410, either physically, temporally, or both. In some embodiments, timeline 420 may not be displayed at all times. As one example, timeline 420 may automatically switch to a collapsed or hidden view while a user is viewing the video and may return to the expanded view shown in FIG. 4 when the user takes an action to interact with timeline 420. For example, user may move a mouse pointer while viewing the video, move the mouse pointer over the collapsed timeline, move the mouse pointer to a particular region, click or tap the video playback region, or perform any other actions that may indicate an intent to interact with timeline 420. As discussed above, timeline 420 may be displayed in various other locations relative to video playback region 410, including on a top portion of video playback region 410, above or below video playback region 410, or within control bar 612. In some embodiments, timeline 420 may be displayed separately from a video progress bar. For example, a separate video progress bar, including position indicator 424 and colored region 422, may be displayed in control bar 412 and timeline 420 may be a separate timeline of events associated with a surgical procedure. In such embodiments, timeline 420 may not have the same scale or range of time as the video or the video progress bar. For example, the video progress bar may represent the time scale and range of the video, whereas timeline 420 may represent the timeframe of the surgical procedure, which may not be the same (e.g., where the video comprises a surgical summary, as discussed in detail above). In some embodiments, video playback region 410 may include a search icon 440, which may allow a user to search for video footage, for example, through a user interface. The surgical timeline shown in FIG. 4 is provided by way of example only, and one skilled in the art would appreciate various other configurations that may be used.

Embodiments of the present disclosure may further include enabling a surgeon, while viewing playback of the at least one video to select one or more markers on the surgical timeline, and thereby cause a display of the video to skip to a location associated with the selected marker. As used herein, “playback” may include any presentation of a video in which one or more frames of the video are displayed to the user. Typically, playback will include sequentially displaying the images to reproduce moving images and/or sounds, however playback may also include the display of individual frames.

Consistent with the disclosed embodiments, a “marker” may include any visual indicator associated with location within the surgical timeline. As described above, the location may refer to any particular position within a video. For example, the location may be a particular frame or range of frames in the video, a particular timestamp, or any other indicator of position within the video. Markers may be represented on the timeline in various ways. In some embodiments, the markers may be icons or other graphic representations displayed along the timeline at various locations. The markers may be displayed as lines, bands, dots, geometric shapes (such as diamonds, squares, triangles, or any other shape), bubbles, or any other graphical or visual representation. In some embodiments, the markers may be text-based. For example, the markers may include textual information, such as a name, a description, a code, a timestamp, and so forth. In another example, the surgical timeline may be displayed as a list, as described above. Accordingly, the markers may include text-based titles or descriptions referring to a particular location of the video. Markers 432, 434, and 436 are shown by way of example in FIG. 4. The markers may be represented as callout bubbles, including an icon indicating the type of marker associated with the location. The markers may point to a particular point along timeline 420 indicating the location in the video.

Selection of the marker may include any action by a user directed towards a particular marker. In some embodiments, selecting the marker may include clicking on or tapping the marker through a user interface, touching the marker on a touch sensitive screen, glancing at the marker through smart glasses, indicating the marker through a voice interface, indicating the marker with a gesture, or undertaking any other action that causes the marker to be selected. Selection of the marker may thereby cause a display of the video to skip to a location associated with the selected marker. As used herein, skipping may include selectively displaying a particular frame within a video. This may include stopping display of a frame at a current location in the video (for example, if the video is currently playing) and displaying a frame at the location associated with the selected marker. For example, if a user clicks on or otherwise selects marker 432, as shown in FIG. 4, a frame at the location associated with marker 432 may be displayed in video playback region 410. In some embodiments, the video may continue playing from that location. Position indicator 424 may move to a position within timeline 420 associated with marker 432 and colored region 422 may be updated accordingly. While the present embodiment is described as enabling a surgeon to select the one or more markers, it is understood that this is an example only, and the present disclosure is not limited to any form of user. Various other users may view and interact with the overlaid timeline, including a surgical technician, a nurse, a physician's assistant, an anesthesiologist, a doctor, or any other healthcare professional, as well as a patient, an insurer, a medical student, and so forth. Other examples of users are provided herein.

In accordance with embodiments of the present disclosure, the markers may be automatically generated and included in the timeline based on information in the video at a given location. In some embodiments, computer analysis may be used to analyze frames of the video footage and identify markers to include at various locations in the timeline. Computer analysis may include any form of electronic analysis using a computing device. In some embodiments, computer analysis may include using one or more image recognition algorithms to identify features of one or more frames of the video footage. Computer analysis may be performed on individual frames, or may be performed across multiple frames, for example, to detect motion or other changes between frames. In some embodiments computer analysis may include object detection algorithms, such as Viola-Jones object detection, scale-invariant feature transform (SIFT), histogram of oriented gradients (HOG) features, convolutional neural networks (CNN), or any other forms of object detection algorithms. Other example algorithms may include video tracking algorithms, motion detection algorithms, feature detection algorithms, color-based detection algorithms, texture based detection algorithms, shape based detection algorithms, boosting based detection algorithms, face detection algorithms, or any other suitable algorithm for analyzing video frames. In one example, a machine learning model may be trained using training examples to generate markers for videos, and the trained machine learning model may be used to analyze the video and generate markers for that video. Such generated markers may include locations within the video for the marker, type of the marker, properties of the marker, and so forth. An example of such training example may include a video clip depicting at least part of a surgical procedure, together with a list of desired markers to be generated, possibly together with information for each desired marker, such as a location within the video for the marker, a type of the marker, properties of the marker, and so forth.

This computer analysis may be used to identify surgical phases, intraoperative events, event characteristics, and/or other features appearing in the video footage. For example, in some embodiments, computer analysis may be used to identify one or more medical instruments used in a surgical procedure, for example as described above. Based on identification of the medical instrument, a particular intraoperative event may be identified at a location in the video footage associated with the medical instrument. For example, a scalpel or other instrument may indicate that an incision is being made and a marker identifying the incision may be included in the timeline at this location. In some embodiments, anatomical structures may be identified in the video footage using the computer analysis, for example as described above. For example, the disclosed methods may include identifying organs, tissues, fluids or other structures of the patient to determine markers to include in the timeline and their respective locations. In some embodiments, locations for video markers may be determined based on an interaction between a medical instrument and the anatomical structure, which may indicate a particular intraoperative event, type of surgical procedure, event characteristic, or other information useful in identifying marker locations. For example, visual action recognition algorithms may be used to analyze the video and detect the interactions between the medical instrument and the anatomical structure. Other examples of features that may be detected in video footage for placing markers may include, motions of a surgeon or other medical professional, patient characteristics, surgeon characteristics or characteristics of other medical professionals, sequences of operations being performed, timings of operations or events, characteristics of anatomical structures, medical conditions, or any other information that may be used to identify particular surgical procedures, surgical phases, intraoperative events, and/or event characteristics appearing in the video footage.

In some embodiments, marker locations may be identified using a trained machine learning model. For example, a machine learning model may be trained using training examples, each training example may include video footage known to be associated with surgical procedures, surgical phases, intraoperative events, and/or event characteristics, together with labels indicating locations within the video footage. Using the trained machine learning model, similar phases and events may be identified in other video footage for the determining marker locations. Various machine learning models may be used, including a logistic regression model, a linear regression model, a regression model, a random forest model, a K-Nearest Neighbor (KNN) model, a K-Means model, a decision tree, a cox proportional hazards regression model, a Naïve Bayes model, a Support Vector Machines (SVM) model, a gradient boosting algorithm, artificial neural networks (such as deep neural networks, convolutional neural networks, etc.) or any other form of machine learning model or algorithm.

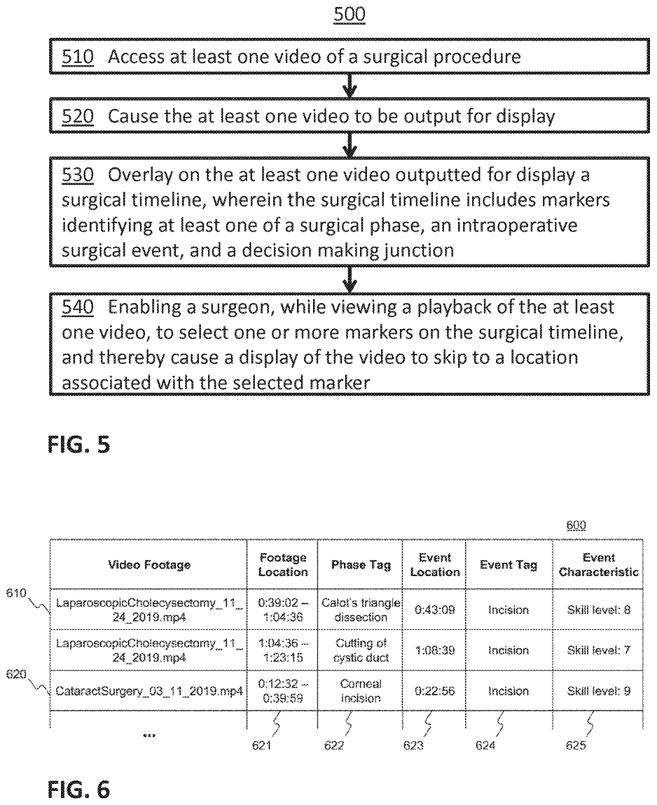

In some embodiments, video markers may be identified in conjunction with the video indexing techniques discussed above. As described above, video footage may be indexed based on surgical phases, intraoperative events, and/or event characteristics identified in the video footage. This information may be stored in a data structure, such as data structure 600, as described in reference to FIG. 6. The data structure may include footage locations and/or event locations associated with phases and events within the video footage. In some embodiments, the markers displayed in the timeline may correspond to these locations in the video. Accordingly any of the techniques or processes described above for indexing video footage may similarly apply to determining marker locations for presenting in a timeline.

According to various exemplary embodiments of the present disclosure, the markers may be coded by at least one of a color or a criticality level. The coding of a marker may be any indicator of a type, property, or characteristic of the marker. The coding may be useful for a user in visually determining which locations of the video may be of interest. Where the marker is coded by color, the color of the marker displayed on the surgical timeline may indicate the property or characteristic of the marker based on a predefined color scheme. For example, the marker may have a different color depending on what type of intraoperative surgical event the marker represents. In some example embodiments, markers associated with an incision, an excision, a resection, a ligation, a graft, or various other events may each be displayed with a different color. In other embodiments, intraoperative adverse events may be associated with one color (e.g., red), where planned events may be associated with another color (e.g., green). In some embodiments, color scales may be used. For example, the severity of an adverse event may be represented by on a color scale ranging from yellow to red, or other suitable color scales.

In some embodiments, the location and/or size of the marker may be associated with a criticality level. The criticality level may represent the relative importance of an event, action, technique, phase or other occurrence identified by the marker. Accordingly, as used herein, the term “criticality level” refers to any measure of an immediate need for an action to prevent hazardous result within a surgical procedure. For example, criticality level may include a numerical measure (such as “1.12”, “3.84”, “7”, “−4.01”, etc.), for example within a particular range of values. In another example, criticality level may include finite number of discrete levels (such as “Level 0”, “Level 1”, “Level 2”, “High Criticality”, “Low Criticality”, “Non Critical”, etc.).

While color is provided as one example for distinguishing marker appearance to represent information, various other techniques may be used. For example, markers may have varying sizes, shapes, positions, orientations, font size, font types, font colors, marker animations, or other visual properties. In some embodiments, markers may be associated with different icons depending on the type of event, action, or phase with which they are associated. For example, as shown in FIG. 4, marker 432, which may be associated with a decision junction, may have a different icon than marker 434, which may be associated with another type of event, such as a complication. The icon may represent the type of intraoperative event associated with that location. For example, marker 436 may indicate that an incision occurs at this location in the video. The icons (or other visual properties) may be used to distinguish between unplanned events and planned events, types of errors (e.g., miscommunication errors, judgment errors, or other forms of errors), specific adverse events that occurred, types of techniques being performed, the surgical phase being performed, locations of intraoperative surgical events (e.g., in the abdominal wall, etc.), a surgeon performing the procedure, an outcome of the surgical procedure, or various other information.

In some exemplary embodiments, the one or more markers may include a decision making junction marker corresponding to a decision making junction of the surgical procedure. In some embodiments, such decision making junction markers maybe visually distinct from other forms or types of markers. As an illustrative example, the decision making junction marker may have an icon indicating the location is associated with a decision making junction, as shown in FIG. 4 by marker 432. As used herein, a decision making junction may refer to any part of a procedure in which a decision is made, or in which a decision of a selected type of decisions or of a plurality of selected types of decisions is made. For example, the decision making junction marker may indicate a location of a video depicting a surgical procedure where multiple courses of action are possible, and a surgeon opts to follow one course over another. For example, the surgeon may decide whether to depart from a planned surgical procedure, to take a preventative action, to remove an organ or tissue, to use a particular instrument, to use a particular surgical technique, or any other intraoperative decisions a surgeon may encounter. In one example, a decision making junction may refer to a part of a procedure in which a decision that has significant effect on an outcome of the procedure is made. In another example, decision making junction may refer to a part of a procedure in which a decision that has no clear decision making guidelines has to be made. In yet another example, a decision making junction may refer to a part of a procedure in which a surgeon is faced with two or more viable alternatives, and where choosing the better alternative of the two or more viable alternatives (for example, the alternative that is predicted to reduce a particular risk, the alternative that is predicted to improve outcome, the alternative that is predicted to reduce cost, etc.) is based on at least a particular number of factors (for example, is based on at least two factors, on at least five factors, on at least ten factors, on at least one hundred factors, and so forth). In an additional example, decision making junction may refer to a part of a procedure in which a surgeon is faced with a decision of a particular type, and where the particular type is included in a group of selected decision types.

The decision making junction may be detected using the computer analysis described above. In some embodiments, video footage may be analyzed to identify particular actions or sequences of actions performed by a surgeon that may indicate a decision has been made. For example, if the surgeon pauses during a procedure, begins to use a different medical device, or changes to a different course of action, this may indicate a decision has been made. In some embodiments, the decision making junction may be identified based on a surgical phase or intraoperative event identified in the video footage at that location. For example, an adverse event, such as a bleed, may be detected which may indicate a decision must be made on how to address the adverse event. As another example, a particular phase of a surgical procedure may be associated with multiple possible courses of action. Accordingly, detecting this surgical phase in the video footage may indicate a decision making junction. In some embodiments, a trained machine learning model may be used to identify the decision making junction. For example, a machine learning model may be trained using training examples to detect decision making junctions in videos, and the trained machine learning model may be used to analyze the video and detect the decision making junction. An example of such training example may include a video clip, together with a label indicating locations of decision making junctions within the video clip, or together with a label indicating an absent of decision making junctions in the video clip.

The selection of the decision making junction marker may enable the surgeon to view two or more alternative video clips from two or more corresponding other surgical procedures, thereby enabling the viewer to compare alternative approaches. Alternative video clips may be any video clips illustrating a procedure other than one currently being displayed to the user. Such an alternative may be drawn from other video footage not included in the current video being output for display. Alternatively, if the current video footage includes a compilation of differing procedures, the alternative footage may be drawn from a differing location of the current video footage being displayed. The other surgical procedures may be any surgical procedure other than the specific procedure depicted in the current video being output for display. In some embodiments, the other surgical procedures may be the same type of surgical procedure depicted in the video being output for display, but performed at different times, on different patients, and/or by different surgeons. In some embodiments, the other surgical procedures may not be the same type of procedure but may share the same or similar decision making junctions as the one identified by the decision making junction marker. In some embodiments, the two or more video clips may present differing conduct. For example, the two or more video clips may represent an alternate choice of action than the one taken in the current video, as represented by the decision making junction marker.

The alternative video clips may be presented in various ways. In some embodiments, selecting the decision making junction marker may automatically cause display of the two or more alternative video clips. For example, one or more of the alternative video clips may be displayed in video playback region 410. In some embodiments, the video playback region may be split or divided to show one or more of the alternative video clips and/or the current video. In some embodiments, the alternative video clips may be displayed in another region, such as above, below, or to the side of video playback region 410. In some embodiments, the alternative video clips may be displayed in a second window, on another screen, or in any other space other than playback region 410. According to other embodiments, selecting the decision marker may open a menu or otherwise display options for viewing the alternative video clips. For example, selecting the decision naming marker may pop up an alternative video menu containing depictions of the conduct in the associated alternative video clips. The alternative video clips may be presented as thumbnails, text-based descriptions, video previews (e.g., playing a smaller resolution version or shortened clip), or the like. The menu may be overlaid on the video, may be displayed in conjunction with the video, or may be displayed in a separate area.

In accordance with embodiments of the present disclosure, the selection of the decision making junction marker may cause a display of one or more alternative possible decisions related to the selected decision making junction marker. Similar to the alternative videos, the alternative possible decisions may be overlaid on the timeline and/or video, or may be displayed in a separate region, such as above, below and/or to the side of the video, in a separate window, on a separate screen, or in any other suitable manner. The alternative possible decisions may be a list of alternative decisions the surgeon could have made at the decision making junction. The list may also include images (e.g., depicting alternative actions), flow diagrams, statistics (e.g., success rates, failure rates, usage rates, or other statistical information), detailed descriptions, hyperlinks, or other information associated with the alternative possible decisions that may be relevant to the surgeon viewing the playback. Such a list may be interactive, enabling the viewer to select an alternative course of action from the list and thereby cause video footage of the alternative course of action to be displayed.

Further, in some embodiments, one or more estimated outcomes associated with the one or more alternative possible decisions may be displayed in conjunction with the display of the one or more alternative possible decisions. For example, the list of alternative possible decisions may include estimated outcomes of each of the alternative possible decisions. The estimated outcomes may include an outcome that is predicted to occur were the surgeon to have taken the alternative possible decision. Such information may be helpful for training purposes. For example, the surgeon may be able to determine that a more appropriate action could have been taken than the one in the video and may plan future procedures accordingly. In some embodiments, each of the alternative possible decisions may be associated with multiple estimated outcomes and a probability of each may be provided. The one or more estimated outcomes may be determined in various ways. In some embodiments, the estimated outcomes may be based on known probabilities associated with the alternative possible decisions. For example, aggregated data from previous surgical procedures with similar decision making junctions may be used to predict the outcome of the alternative possible decisions associated with the marker. In some embodiments, the probabilities and/or data may be tailored to one or more characteristics or properties of the current surgical procedure. For example, patient characteristics (such as a patient's medical condition, age, weight, medical history, or other characteristics), surgeon skill level, difficulty of the procedure, type of procedure, or other factors may be considered in determining the estimated outcomes. Other characteristics may also be analyzed, including the event characteristics described above with respect to video indexing.

In accordance with the present disclosure, the decision making junction of the surgical procedure may be associated with a first patient, and the respective similar decision making junctions may be selected from past surgical procedures associated with patients with similar characteristics to the first patient. The past surgical procedures may be preselected or automatically selected based on similar estimated outcomes as the respective similar decision making junctions, or because of similarities between the patient in the current video with the patient's in the past surgical procedures. These similarities or characteristics may include a patient's gender, age, weight, height, physical fitness, heart rate, blood pressure, temperature, whether the patient exhibits a particular medical condition or disease, medical treatment history, or any other traits or conditions that may be relevant.

Similarly, in some embodiments, the decision making junction of the surgical procedure may be associated with a first medical professional, and the respective similar past decision making junctions may be selected from past surgical procedures associated with medical professionals with similar characteristics to the first medical professional. These characteristics may include, but are not limited to, the medical professional's age, medical background, experience level (e.g., the number of times the surgeon has performed this or similar surgical procedures, the total number of surgical procedures the surgeon has performed, etc.), skill level, training history, success rate for this or other surgical procedures, or other characteristics that may be relevant.

In some exemplary embodiments, the decision making junction of the surgical procedure is associated with a first prior event in the surgical procedure, and the similar past decision making junctions are selected from past surgical procedures including prior events similar to the first prior event. In one example, prior events may be determined to be similar to the first prior event based on, for example, the type of the prior events, characteristics of the prior events, and so forth. For example, a prior event may be determined as similar to the first prior event when a similarity measure between the two is above a selected threshold. Some non-limiting examples of such similarity measures are described above. The occurrence and/or characteristics of the prior event may be relevant for determining estimated outcomes for the alternative possible decisions. For example, if the surgeon runs into complications with a patient, the complications may at least partially be determinative of the most appropriate outcome, whereas a different outcome may be appropriate in absence of the complications. The first prior event may include, but is not limited to, any of the intraoperative events described in detail above. Some non-limiting characteristics of the first prior may include any of the event characteristics described above. For example, the first prior event may include an adverse event or complication, such as bleeding, mesenteric emphysema, injury, conversion to unplanned open, incision significantly larger than planned, hypertension, hypotension, bradycardia, hypoxemia, adhesions, hernias, atypical anatomy, dural tears, periorator injury, arterial occlusions, and so forth. The first prior event may also include positive or planned events, such as a successful incision, administration of a drug, usage of a surgical instrument, an excision, a resection, a ligation, a graft, suturing, stitching, or any other event.

In accordance with the present disclosure, the decision making junction of the surgical procedure may be associated with a medical condition, and the respective similar decision making junctions may be selected from past surgical procedures associated with patients with similar medical conditions. The medical conditions may include any condition of the patient related to the patient's health or well-being. In some embodiments, the medical condition may be the condition being treated by the surgical procedure. In other embodiments, the medical condition may be a separate medical condition. The medical condition may be determined in various ways. In some embodiments, the medical condition may be determined based on data associated with the plurality of videos. For example, the video may be tagged with information including the medical condition. In other embodiments, the medical condition may be determined by an analysis of the at least one video and may be based on an appearance of an anatomical structure appearing in the at least one video. For example, the color of a tissue, the relative color of one tissue with respect to the color of another tissue, size of an organ, relative size of one organ with respect to a size of another organ, appearance of a gallbladder or other organ, presence of lacerations or other marks, or any other visual indicators associated with an anatomical structure, may be analyzed to determine the medical condition. In one example, a machine learning model may be trained using training examples to determine medical conditions from videos, and the trained machine learning model may be used to analyze the at least one video footage and determine the medical condition. An example of such training example may include a video clip of a surgical procedure, together with a label indicating one or more medical conditions.

In some aspects of the present disclosure, information related to a distribution of past decisions made in respective similar past decision making junctions may be displayed in conjunction with the display of the alternative possible decisions. For example, as described above, a particular decision making junction may be associated with multiple possible decisions for a course of action. The past decisions may include decisions that were made by surgeons in previous surgical procedures when faced with the same or similar decision making junction. For example, each of the past decisions may correspond to one of the alternate possible decisions described above. Accordingly, as used herein, respective similar past decision making junctions refers to the decision making junction that occurred in the past surgical procedure when the past decision was made. In some embodiments, the respective similar past decision making junctions may be the same as the decision making junction identified by the marker. For example, if the decision making junction is an adverse event, such as a bleed, the past decisions may correspond to how other surgeons have addressed the bleed in previous surgical procedures. In other embodiments, the decision making junction may not be identical, but may be similar. For example, the possible decisions made by surgeons encountering a dural tear may be similar to other forms of tears and, accordingly, a distribution of past decisions associated with a dural tear may be relevant to the other forms of tears. The past decisions may be identified by analyzing video footage, for example, using the computer analysis techniques described above. In some embodiments, the past decisions may be indexed using the video indexing techniques described above, such that they can be readily accessed for displaying a distribution of past decisions. In one example, the distribution may include a conditional distribution, for example presenting a distribution of past decisions made in respective similar past decision making junctions that has a common property. In another example, the distribution may include an unconditional distribution, for example presenting a distribution of past decisions made in all respective similar past decision making junctions.

The displayed distribution may indicate how common each of the possible decisions were among the other alternative possible decisions associated with the respective similar past decision making junctions. In some embodiments, the displayed distribution may include a number of times each of the decisions was made. For example, a particular decision making junction may have three alternative possible decisions: decision A, decision B, and decision C. Based on the past decisions made in similar decision making junctions, the number of times each of these alternative possible decisions has been performed may be determined. For example, decision A may have been performed 167 times, decision B may have been performed 47 times, and decision C may have been performed 13 times. The distribution may be displayed as a list of each of the alternative possible decisions, along with the number of times they have been performed. The displayed distribution may also indicate the relative frequency of each of the decisions, for example, by displaying ratios, percentages, or other statistical information. For example, the distribution may indicate that decisions A, B and C have been performed in 73.6%, 20.7% and 5.7% of past decisions, respectively. In some embodiments, the distribution may be displayed as a graphical representation of the distribution, such as a bar graph, a histogram, a pie chart, a distribution curve, or any other graphical representation that may be used to show distribution.

In some embodiments, only a subset of the decisions may be displayed. For example, only the most common decisions may be displayed based on the number of times the decision was made (e.g., exceeding a threshold number of times, etc.). Various methods described above for identifying the similar past decision making junctions may be used, including identifying surgical procedures associated with similar medical conditions, patient characteristics, medical professional characteristics, and/or prior events.

In some embodiments, the one or more estimated outcomes may be a result of an analysis of a plurality of videos of past surgical procedures including respective similar decision making junctions. For example, a repository of video footage may be analyzed using various computer analysis techniques, such as the object and/or motion detection algorithms described above, to identify videos including decision making junctions that are the same as or share similar characteristics with the decision making junction identified by the marker. This may include identifying other video footage having the same or similar surgical phases, intraoperative surgical events, and/or event characteristics as those that were used to identify the decision making junction in the video presented in the timeline. The outcomes of the alternative possible decisions may be estimated based on the outcomes in the past surgical procedures. For example, if a particular method of performing a suture consistently results in a full recovery by the patient, this outcome may be estimated for this possible decision and may be displayed on the timeline.

In some exemplary embodiments, the analysis may include usage of an implementation of a computer vision algorithm. The computer vision algorithm may be the same as or similar to any of the computer vision algorithms described above. One example of such computer algorithm may include the object detection and tracking algorithms described above. Another example of such computer vision algorithm may include usage of a trained machine learning model. Other non-limiting examples of such computer vision algorithm are described above. For example, if the decision making junction marker was identified based on a particular adverse event occurring in the video, other video footage having the same or similar adverse events may be identified. The video footage may further be analyzed to determine an outcome of the decision made in past surgical video. This may include the same or similar computer analysis techniques described above. In some embodiments, this may include analyzing the video to identify the result of the decision. For example, if the decision making junction is associated with an adverse event associated with an anatomical structure, such as a tear, the anatomical structure may be assessed at various frames after the decision to determine whether the adverse event was remediated, how quickly it was remediated, whether additional adverse events occurred, whether the patient survived, or other indicators of the outcome.

In some embodiments, additional information may also be used to determine the outcome. For example, the analysis may be based on one or more electronic medical records associated with the plurality of videos of past surgical procedures. For example, determining the outcome may include referencing an electronic medical record associated with the video in which a particular decision was made to determine whether the patient recovered, how quickly the patient recovered, whether there were additional complications, or the like. Such information may be useful in predicting the outcome that may result at a later time, outside of the scope of the video footage. For example, the outcome may be several days, weeks, or months after the surgical procedure. In some embodiments, the additional information may be used to inform the analysis of which videos to include in the analysis. For example, using information gleaned from the medical records, videos sharing similar patient medical history, disease type, diagnosis type, treatment history (including past surgical procedures), healthcare professional identities, healthcare professional skill levels, or any other relevant data may be identified. Videos sharing these or other characteristics may provide a more accurate idea of what outcome can be expected for each alternative possible decision.

The similar decision making junctions may be identified based on how closely they correlate to the current decision making junction. In some embodiments, the respective similar decision making junctions may be similar to the decision making junction of the surgical procedure according to a similarity metric. The metric may be any value, classification, or other indicator of how closely the decision making junctions are related. Such a metric may be determined based on the computer vision analysis in order to determine how closely the procedures or techniques match. The metric may also be determined based on the number of characteristics the decision making junctions have in common and the degree to which the characteristics match. For example, two decision making junctions with patients having similar medical conditions and physical characteristics may be assigned a higher similarity based on the similarity metric than two more distinctive patients. Various other characteristics and/or considerations may also be used. Additionally or alternatively, the similarity metric may be based on any similarity measure, such as the similarity measures described above. For example, the similarity metric may be identical to the similarity measure, may be a function of the similarity measure, and so forth.

Various other marker types may be used in addition to or instead of decision making junction markers. In some embodiments, the markers may include intraoperative surgical event markers, which may be associated with locations in the video associated with the occurrence of an interoperative event. Examples of various intraoperative surgical events that may be identified by the markers are provided throughout the present disclosure, including in relation to the video indexing described above. In some embodiments, the intraoperative surgical event markers may be generic markers, indicating that an intraoperative surgical event occurred at that location. In other embodiments, the intraoperative surgical event markers may identify a property of the intraoperative surgical event, including the type of the event, whether the event was an adverse event, or any other characteristic. Example markers are shown in FIG. 4. As an illustrative example, the icon shown for marker 434 may be used to represent a generic intraoperative surgical event marker. Marker 436 on the other hand, may represent a more specific intraoperative surgical event marker, such as identifying that an incision occurred at that location. The markers shown in FIG. 4 are provided by way of example, and various other forms of markers may be used.

These intraoperative surgical event markers may be identified automatically, as described above. Using the computer analysis methods described above, medical instruments, anatomical structures, surgeon characteristics, patient characteristics, event characteristics, or other features may be identified in the video footage. For example, the interaction between an identified medical instrument and an anatomical structure may indicate that an incision, a suturing, or other intraoperative event is being performed. In some embodiments, the intraoperative surgical event markers may be identified based on information provided in a data structure, such as data structure 600 described above in reference to FIG. 6.

Consistent with the disclosed embodiments, selection of an intraoperative surgical event marker may enable the surgeon to view alternative video clips from differing surgical procedures. In some embodiments, the alternative video clips may present differing ways in which a selected intraoperative surgical event was handled. For example, in the current video the surgeon may perform an incision or other action according to one technique. Selecting the intraoperative surgical event markers may allow the surgeon to view alternative techniques that may be used to perform the incision or other action. In another example, the intraoperative surgical event may be an adverse event, such as a bleed, and the alternative video clips may depict other ways surgeons have handled the adverse event. In some embodiments, where the markers relate to intraoperative surgical events, the selection of an intraoperative surgical event marker may enable the surgeon to view alternative video clips from differing surgical procedures. For example, the differing surgical procedures may be of a different type (such as a laparoscopic surgery versus thoracoscopic surgery) but may still include the same or similar intraoperative surgical events. The surgical procedures may also differ in other ways, including differing medical conditions, differing patient characteristics, differing medical professionals, or other distinctions. Selecting the intraoperative surgical event marker may allow the surgeon to view alternative video clips from the differing surgical procedures.

The alternative video clips may be displayed in various ways, similar to other embodiments described herein. For example, selecting the intraoperative surgical event markers may cause a menu to be displayed, from which the surgeon may select the alternative video clips. The menu may include descriptions of the differing ways in which the selected intraoperative surgical event was handled, thumbnails of the video clips, previews of the video clips, and/or other information associated with the video clips, such as the dates they were recorded, the type of surgical procedure, a name or identity of a surgeon performing the surgical procedure, or any other relevant information.

In accordance with some embodiments of the present disclosure, the at least one video may include a compilation of footage from a plurality of surgical procedures, arranged in procedural chronological order. Procedural chronological order may refer to the order events occur relative to a surgical procedure. Accordingly, arranging a compilation of footage in procedural chronological order may include arranging the different events from differing patients in the order in which they would have occurred if the procedure had been conducted on a single patient. In other words, although compiled from various surgeries on differing patients, playback of the compilation will display the footage in the order the footage would appear within the surgical procedure. In some embodiments, the compilation of footage may depict complications from the plurality of surgical procedures. In such embodiments, the one or more markers may be associated with the plurality of surgical procedures and may be displayed on a common timeline. Thus, although a viewer interacts with a single timeline, the video footage presented along the timeline may be derived from differing procedures and/or differing patients. Example complications that may be displayed are described above with respect to video indexing.

FIG. 5 is a flowchart illustrating an example process 500 for reviewing surgical videos, consistent with the disclosed embodiments. Process 500 may be performed by at least one processor, such as one or more microprocessors. In some embodiments, process 500 is not necessarily limited to the steps illustrated, and any of the various embodiments described herein may also be included in process 500. At step 510, process 500 may include accessing at least one video of a surgical procedure, for example as described above. The at least one video may include video footage from a single surgical procedure or may be a compilation of footage from a plurality of procedures, as previously discussed. Process 500 may include causing the at least one video to be output for display in step 520. As described above, causing the at least one video to be output for display may include sending a signal for causing display of the at least one video on a screen or other display device, storing the at least one video in a location accessible to another computing device, transmitting the at least one video, or any other process or steps that may cause the video to be displayed.

At step 530, process 500 may include overlaying on the at least one video outputted for display a surgical timeline, wherein the surgical timeline includes markers identifying at least one of a surgical phase, an intraoperative surgical event, and a decision making junction. In some embodiments, the surgical timeline may be represented as a horizontal bar displayed along with the video. The markers may be represented as shapes, icons, or other graphical representations along the timeline. FIG. 4 provides an example of such an embodiment. In other embodiments, the timeline may be a text-based list of phases, events, and/or decision making junctions in chronological order. The markers may similarly be text-based and may be included in the list.

Step 540 may include enabling a surgeon, while viewing playback of the at least one video, to select one or more markers on the surgical timeline, and thereby cause a display of the video to skip to a location associated with the selected marker. In some embodiments, the surgeon may be able to view additional information about the event or occurrence associated with the marker, which may include information from past surgical procedures. For example, the markers may be associated with an intraoperative surgical event and selecting the marker may enable the surgeon to view alternative video clips of past surgical procedures associated with the intraoperative surgical event. For example, the surgeon may be enabled to view clips from other surgeries where a similar intraoperative surgical event was handled differently, where a different technique was used, or where an outcome varied. In some embodiments, the marker may be a decision making junction marker, representing a decision that was made during the surgical procedure. Selecting the decision making junction marker may enable the surgeon to view information about the decision, including alternative decisions. Such information may include videos of past surgical procedures including similar decision making junctions, a list or distribution of alternate possible decisions, estimated outcomes of the alternate possible decisions, or any other relevant information. Based on the steps described in process 500, the surgeon or other users may be able to more effectively and more efficiently review surgical videos using the timeline interface.

In preparing for a surgical procedure, it is often beneficial for surgeons to review videos of similar surgical procedures that have been performed. It may be too cumbersome and time consuming, however, for a surgeon to identify relevant videos or portions of videos in preparing for a surgical procedure. Therefore, there is a need for unconventional approaches that efficiently, effectively index surgical video footage based on contents of the footage such that it may be easily accessed and reviewed by a surgeon or other medical professional.

Aspects of this disclosure may relate to video indexing, including methods, systems, devices, and computer readable media. For example, surgical events within surgical phases may be automatically detected in surgical footage. Viewers may be enabled to skip directly to an event, to view only events with specified characteristics, and so forth. In some embodiments, a user may specify within a surgical phase (e.g., a dissection) an event (e.g., inadvertent injury to an organ) having a characteristic (e.g., a particular complication), so that the user may be presented with video clips of one or more events sharing that characteristic.

For ease of discussion, a method is described below, with the understanding that aspects of the method apply equally to systems, devices, and computer readable media. For example, some aspects of such a method may occur electronically over a network that is either wired, wireless, or both. Other aspects of such a method may occur using non-electronic means. In a broadest sense, the method is not limited to particular physical and/or electronic instrumentalities, but rather may be accomplished using many differing instrumentalities.

Consistent with disclosed embodiments, a method may involve accessing video footage to be indexed, the video footage to be indexed including footage of a particular surgical procedure. As used herein, video may include any form of recorded visual media including recorded images and/or sound. For example, a video may include a sequence of one or more images captured by an image capture device, such as cameras 115, 121, 123, and/or 125, as described above in connection with FIG. 1. The images may be stored as individual files or may be stored in a combined format, such as a video file, which may include corresponding audio data. In some embodiments, video may be stored as raw data and/or images output from an image capture device. In other embodiments the video may be processed. For example, video files may include Audio Video Interleave (AVI), Flash Video Format (FLV), QuickTime File Format (MOV), MPEG (MPG, MP4, M4P, etc.), Windows Media Video (WMV), Material Exchange Format (MXF), uncompressed format, lossy compressed format, lossless compressed format, or any other suitable video file formats.

Video footage may refer to a length of video that has been captured by an image capture device. In some embodiments, video footage may refer to a length of video that includes a sequence of images in the order they were originally captured in. For example, video footage may include video that has not been edited to form a video compilation. In other embodiments, video footage may be edited in one or more ways, such as to remove frames associated with inactivity, or to otherwise compile frames not originally captured sequentially. Accessing the video footage may include retrieving video footage from a storage location, such as a memory device. The video footage may be accessed from a local memory, such as a local hard drive, or may be accessed from a remote source, for example, through a network connection. Consistent with the present disclosure, indexing may refer to a process for storing data such that it may be retrieved more efficiently and/or effectively. Indexing video footage may include associating one or more properties or indicators with the video footage such that the video footage may be identified based on the properties or indicators.

A surgical procedure may include any medical procedure associated with or involving manual or operative procedures on a patient's body. Surgical procedures may include cutting, abrading, suturing, or other techniques that involve physically changing body tissues and organs. Some examples of such surgical procedures may include a laparoscopic surgery, a thoracoscopic procedure, a bronchoscopic procedure, a microscopic procedure, an open surgery, a robotic surgery, an appendectomy, a carotid endarterectomy, a carpal tunnel release, a cataract surgery, a cesarean section, a cholecystectomy, a colectomy (such as a partial colectomy, a total colectomy, etc.), a coronary angioplasty, a coronary artery bypass, a debridement (for example of a wound, a burn, an infection, etc.), a free skin graft, a hemorrhoidectomy, a hip replacement, a hysterectomy, a hysteroscopy, an inguinal hernia repair, a knee arthroscopy, a knee replacement, a mastectomy (such as a partial mastectomy, a total mastectomy, a modified radical mastectomy, etc.), a prostate resection, a prostate removal, a shoulder arthroscopy, a spine surgery (such as a spinal fusion, a laminectomy, a foraminotomy, a discectomy, a disk replacement, an interlaminar implant, etc.), a tonsillectomy, a cochlear implant procedure, brain tumor (for example meningioma, etc.) resection, interventional procedures such as percutaneous transluminal coronary angioplasty, transcatheter aortic valve replacement, minimally Invasive surgery for intracerebral hemorrhage evacuation, or any other medical procedure involving some form of incision. While the present disclosure is described in reference to surgical procedures, it is to be understood that it may also apply to other forms of medical procedures, or procedures generally.

In some exemplary embodiments, the accessed video footage may include video footage captured via at least one image sensor located in at least one of a position above an operating table, in a surgical cavity of a patient, within an organ of a patient or within vasculature of a patient. An image sensor may be any sensor capable of recording video. An image sensor located in a position above an operating table may include any image sensor placed external to a patient configured to capture images from above the patient. For example, the image sensor may include cameras 115 and/or 121, as shown in FIG. 1. In other embodiments, the image sensor may be placed internal to the patient, such as, for example, in a cavity. As used herein, a cavity may include any relatively empty space within an object. Accordingly, a surgical cavity may refer to a space within the body of a patient where a surgical procedure or operation is being performed, or where surgical tools are present and/or used. It is understood that the surgical cavity may not be completely empty but may include tissue, organs, blood or other fluids present within the body. An organ may refer to any self-contained region or part of an organism. Some examples of organs in a human patient may include a heart or liver. A vasculature may refer to a system or grouping of blood vessels within an organism. An image sensor located in a surgical cavity, an organ, and/or a vasculature may include a camera included on a surgical tool inserted into the patient.

Aspects of this disclosure may include analyzing the video footage to identify a video footage location associated with a surgical phase of the particular surgical procedure. As used herein with respect to video footages, a location may refer any particular position or range within the video footage. In some embodiments the location may include a particular frame or range of frames of a video. Accordingly, video footage locations may be represented as one or more frame numbers or other identifiers of a video footage file. In other embodiments, the location may refer to a particular time associated with the video footage. For example, a video footage location may refer to a time index or timestamp, a time range, a particular starting time and/or ending time, or any other indicator of position within the video footage. In other embodiments, the location may refer to at least one particular position within at least one frame. Accordingly, video footage locations may be represented as one or more pixels, voxels, bounding boxes, bounding polygons, bounding shapes, coordinates, and so forth.

For the purposes of the present disclosure, a phase may refer to a particular period or stage of a process or series of events. Accordingly, a surgical phase may refer to a particular period or stage of a surgical procedure, as described above. For example, surgical phases of a laparoscopic cholecystectomy surgery may include trocar placement, preparation, calot's triangle dissection, clipping and cutting of cystic duct and artery, gallbladder dissection, gallbladder packaging, cleaning and coagulation of liver bed, gallbladder retraction, and so forth. In another example, surgical phases of a cataract surgery may include preparation, povidone-iodine injection, corneal incision, capsulorhexis, phaco-emulsification, cortical aspiration, intraocular lens implantation, intraocular-lens adjustment, wound sealing, and so forth. In yet another example, surgical phases of a pituitary surgery may include preparation, nasal incision, nose retractor installation, access to the tumor, tumor removal, column of nose replacement, suturing, nose compress installation, and so forth. Some other examples of surgical phases may include preparation, incision, laparoscope positioning, suturing, and so forth.

In some embodiments, identifying the video footage location may be based on user input. User input may include any information provided by a user. As used with respect to video indexing, the user input may include information relevant to identifying the video footage location. For example, a user may input a particular frame number, timestamp, range of times, start times and/or stop times, or any other information that may identify a video footage location. Alternatively, the user input might include entry or selection of a phase, event, procedure, or device used, which input may be associated with particular video footage (e.g., for example through a lookup table or other data structure). The user input may be received through a user interface of a user device, such as a desktop computer, a laptop, a table, a mobile phone, a wearable device, an internet of things (IoT) device, or any other means for receiving input from a user. The interface may include, for example, one or more drop down menus with one or more pick lists of phase names; a data entry field that permits the user to enter the phase name and/or that suggests phase names once a few letters are entered; a pick list from which phase names may be chosen; a group of selectable icons each associated with a differing phase, or any other mechanism that allows users to identify or select a phase. For example, a user may input the phase name through a user interface. In another example, the user input may be received through voice commands and/or voice inputs, and the user input may be processed using speech recognition algorithms. In yet another example, the user input may be received through gestures (such as hand gestures), and the user input may be processed using gesture recognition algorithms.

In some embodiments, identifying the video footage location may include using computer analysis to analyze frames of the video footage. Computer analysis may include any form of electronic analysis using a computing device. In some embodiments, computer analysis may include using one or more image recognition algorithms to identify features of one or more frames of the video footage. Computer analysis may be performed on individual frames, or may be performed across multiple frames, for example, to detect motion or other changes between frames. In some embodiments computer analysis may include object detection algorithms, such as Viola-Jones object detection, scale-invariant feature transform (SIFT), histogram of oriented gradients (HOG) features, convolutional neural networks (CNN), or any other forms of object detection algorithms. Other example algorithms may include video tracking algorithms, motion detection algorithms, feature detection algorithms, color-based detection algorithms, texture based detection algorithms, shape based detection algorithms, boosting based detection algorithms, face detection algorithms, or any other suitable algorithm for analyzing video frames. In one example, a machine learning model may be trained using training examples to identify particular locations within videos, and the trained machine learning model may be used to analyze the video footage and identify the video footage location. An example of such training example may include a video clip together with a label indicating a location within a video clip, or together with a label indicating that no corresponding location is included within the video clip.

In some embodiments, the computer image analysis may include using a neural network model trained using example video frames including previously-identified surgical phases to thereby identify at least one of a video footage location or a phase tag. In other words, frames of one or more videos that are known to be associated with a particular surgical phase may be used to train a neural network model, for example using a machine learning algorithm, using back propagation, using gradient descent optimization, and so forth. The trained neural network model may therefore be used to identify whether one or more video frames are also associated with the surgical phase. Some non-limiting examples of such artificial neural networks may comprise shallow artificial neural networks, deep artificial neural networks, feedback artificial neural networks, feed forward artificial neural networks, autoencoder artificial neural networks, probabilistic artificial neural networks, time delay artificial neural networks, convolutional artificial neural networks, recurrent artificial neural networks, long short term memory artificial neural networks, and so forth. In some embodiments, the disclosed methods may further include updating the trained neural network model based on at least one of the analyzed frames.

In some aspects of the present disclosure, analyzing the video footage to identify the video footage location associated with at least one of the surgical event or the surgical phase may include performing computer image analysis on the video footage to identify at least one of a beginning location of the surgical phase for playback or a beginning of a surgical event for playback. In other words, using the computer analysis techniques discussed above, the disclosed methods may include identifying a location within the video footage where a surgical phase or event begins. For example, the beginning of a surgical event, such as an incision, may be detected using the object and/or motion detection algorithms described above. In other embodiments, the beginning of the incision may be detected based on machine learning techniques. For example, a machine learning model may be trained using video footage and corresponding label indicating known beginning points of an incision or other surgical events and/or procedures. The trained model may be used to identify similar procedure and/or event beginning locations within other surgical video footage.

Some aspects of this disclosure may include generating a phase tag associated with the surgical phase. As used herein, a “tag” may refer to any process or marker by which information is associated with or linked to a set of data. In some embodiments, a tag may be a property of a data file, such as a video file. Accordingly, generating the tag may include writing or overwriting properties within a video file. In some embodiments, generating a tag may include writing information to a file other than the video file itself, for example, by associating the video file with the tag in a separate database. The tag may be expressed as textual information, a numerical identifier, or any other suitable means for tagging. A phase tag may be a tag that identifies a phase of a surgical phase, as described above. In one embodiment, a phase tag may be a marker indicating a location in video where a surgical phase begins, a marker indicating a location in video where a surgical phase ends, a marker indicating a location in video in the middle of a surgical phase, or indicating a range of video encompassing the surgical phase. The tag may be a pointer in the video data itself or may be located in a data structure to permit a lookup of a phase location. The phase tag may include computer readable information for causing display of the phase and may also include human-readable information for identifying the phase to a user. For example, generating a phase tag associated with the surgical phase may include generating a tag including text such as “laparoscope positioning” to indicate the tagged data is associated with that phase of the surgical procedure. In another example, generating a phase tag associated with the surgical phase may include generating a tag including binary encoding of a surgical phase identifier. In some embodiments, generating the phase tag may be based on a computer analysis of video footage depicting the surgical phase. For example, the disclosed methods may include analyzing footage of the surgical phase using the object and motion detection analysis methods described above to determine the phase tag. For example, if it is known that a phase begins or ends using a particular type of medical device or other instrumentality used in a unique way or in a unique order, image recognition may be performed on the video footage to identify a particular phase through image recognition performed to identify the unique use of the instrumentality to identify a particular phase. Generating the phase tag may also include using a trained machine learning model or a neural network model (such as deep neural network, convolutional neural networks, etc.), which may be trained to associate one or more video frames with one or more phase tags. For example, training examples may be fed to a machine learning algorithm to develop a model configured to associate other video footage data with one or more phase tags. An example of such training example may include a video footage together with a label indicating the desired tags or the absent of desired tags corresponding to the video footage. Such label may include an indication of one or more locations within the video footage corresponding to the surgical phase, an indication of a type of the surgical phase, an indication of properties of the surgical phase, and so forth.

A method in accordance with the present disclosure may include associating the phase tag with the video footage location. Any suitable means may be used to associate the phase tag with the video footage location. Such tag may include an indication of one or more locations within the video footage corresponding to the surgical phase, an indication of a type of the surgical phase, an indication of properties of the surgical phase, and so forth. In some embodiments, the video footage location may be included in the tag. For example, the tag may include a timestamp, time range, frame number, or other means for associating the phase tag to the video footage location. In other embodiments, the tag may be associated with the video footage location in a database. For example, the database may include information linking the phase tag to the video footage and to the particular video footage location. The database may include a data structure, as described in further detail below.

Embodiments of the present disclosure may further include analyzing the video footage to identify an event location of a particular intraoperative surgical event within the surgical phase. An intraoperative surgical event may be any event or action that occurs during a surgical procedure or phase. In some embodiments, an intraoperative surgical event may include an action that is performed as part of a surgical procedure, such as an action performed by a surgeon, a surgical technician, a nurse, a physician's assistant, an anesthesiologist, a doctor, or any other healthcare professional. The intraoperative surgical event may be a planned event, such as an incision, administration of a drug, usage of a surgical instrument, an excision, a resection, a ligation, a graft, suturing, stitching, or any other planned event associated with a surgical procedure or phase. In some embodiments, the intraoperative surgical event may include an adverse event or a complication. Some examples of intraoperative adverse events may include bleeding, mesenteric emphysema, injury, conversion to unplanned open surgery (for example, abdominal wall incision), incision significantly larger than planned, and so forth. Some examples of intraoperative complications may include hypertension, hypotension, bradycardia, hypoxemia, adhesions, hernias, atypical anatomy, dural tears, periorator injury, arterial occlusions, and so forth. The intraoperative event may include other errors, including technical errors, communication errors, management errors, judgment errors, decision making errors, errors related to medical equipment utilization, miscommunication, and so forth.

The event location may be a location or range within the video footage associated with the intraoperative surgical event. Similar to the phase location described above, the event location may be expressed in terms of particular frames of the video footage (e.g., a frame number or a range of frame numbers) or based on time information (e.g., a timestamp, a time range, or beginning and end times), or any other means for identifying a location within the video footage. In some embodiments, analyzing the video footage to identify the event location may include using computer analysis to analyze frames of the video footage. The computer analysis may include any of the techniques or algorithms described above. As with phase identification, event identification may be based on a detection of actions and instrumentalities used in a way that uniquely identifies an event. For example, image recognition may identify when a particular organ is incised, to enable marking of that incision event. In another example, image recognition may be used to note the severance of a vessel or nerve, to enable marking of that adverse event. Image recognition may also be used to mark events by detection of bleeding or other fluid loss. In some embodiments, analyzing the video footage to identify the event location may include using a neural network model (such as a deep neural network, a convolutional neural network, etc.) trained using example video frames including previously-identified surgical events to thereby identify the event location. In one example, a machine learning model may be trained using training examples to identify locations of intraoperative surgical events in portions of videos, and the trained machine learning model may be used to analyze the video footage (or a portion of the video footage corresponding to the surgical phase) and identify the event location of the particular intraoperative surgical event within the surgical phase. An example of such training example may include a video clip together with a label indicating a location of a particular event within the video clip, or an absence of such event.

Some aspects of the present disclosure may involve associating an event tag with the event location of the particular intraoperative surgical event. As discussed above, a tag may include any means for associating information with data or a portion of data. An event tag may be used to associate data or portions of data with an event, such as an intraoperative surgical event. Similar to the phase tag, associating the event tag with the event location may include writing data to a video file, for example, to the properties of the video file. In other embodiments, associating the event tag with the event location may include writing data to a file or database associating the event tag with the video footage and/or the event location. Alternatively, associating an event tag with an event location may include recording a marker in a data structure, where the data structure correlates a tag with a particular location or range of locations in video footage. In some embodiments, the same file or database may be used to associate the phase tag to the video footage as the event tag. In other embodiments, a separate file or database may be used.

Consistent with the present disclosure, the disclosed methods may include storing an event characteristic associated with the particular intraoperative surgical event. The event characteristic may be any trait or feature of the event. For example, the event characteristic may include properties of the patient or surgeon, properties or characteristics of the surgical event or surgical phase, or various other traits. Examples of features may include, excessive fatty tissue, an enlarged organ, tissue decay, a broken bone, a displaced disc, or any other physical characteristic associated with the event. Some characteristics may be discernable by computer vision, and others may be discernable by human input. In the latter example, the age or age range of a patient may be stored as an event characteristic. Similarly, aspects of a patient's prior medical history may be stored as an event characteristic (e.g., patient with diabetes). In some embodiments, the stored event characteristic may be used to distinguish intraoperative surgical events from other similar events. For example, a medical practitioner may be permitted to search video footage to identify one or more coronary artery bypass surgeries performed on males over the age of 70 with arrhythmia. Various other examples of stored event characteristics that may be used are provided below.

The stored event characteristic may be determined in various ways. Some aspects of the disclosed methods may involve determining the stored event characteristic based on user input. For example, a user may input the event characteristic to be stored via a user interface similar to what was described above in connection with the selection of a phase or an event. In another example, a user may input the event characteristic to be stored via voice commands. Various examples of such uses are provided below. Other aspects of the disclosed methods may involve determining the stored event characteristic based on a computer analysis of video footage depicting the particular intraoperative surgical event. For example, the disclosed methods may include using various image and/or video analysis techniques as described above to recognize event characteristics based on the video footage. As an illustrative example, the video footage may include a representation of one or more anatomical structures of a patient and an event characteristic identifying the anatomical structures may be determined based on detecting the anatomical structure in the video footage, or based on detecting the interaction between a medical instrument and the anatomical structure. In another example, a machine learning model may be trained using training examples to determine event characteristics from videos, and the trained machine learning model may be used to analyze the video footage and determine the stored event characteristic. An example of such training example may include a video clip depicting an intraoperative surgical event together with a label indicating a characteristic of the event.

Some aspects of the present disclosure may include associating at least a portion of the video footage of the particular surgical procedure with the phase tag, the event tag, and the event characteristic in a data structure that contains additional video footage of other surgical procedures, wherein the data structure also includes respective phase tags, respective event tags, and respective event characteristics associated with one or more of the other surgical procedures. A data structure consistent with this disclosure may include any collection of data values and relationships among them. The data may be stored linearly, horizontally, hierarchically, relationally, non-relationally, uni-dimensionally, multidimensionally, operationally, in an ordered manner, in an unordered manner, in an object-oriented manner, in a centralized manner, in a decentralized manner, in a distributed manner, in a custom manner, in a searchable repository, in a sorted repository, in an indexed repository, or in any manner enabling data access. By way of non-limiting examples, data structures may include an array, an associative array, a linked list, a binary tree, a balanced tree, a heap, a stack, a queue, a set, a hash table, a record, a tagged union, ER model, and a graph. For example, a data structure may include an XML database, an RDBMS database, an SQL database or NoSQL alternatives for data storage/search such as, for example, MongoDB, Redis, Couchbase, Datastax Enterprise Graph, Elastic Search, Splunk, SoIr, Cassandra, Amazon DynamoDB, Scylla, HBase, and Neo4J. A data structure may be a component of the disclosed system or a remote computing component (e.g., a cloud-based data structure). Data in the data structure may be stored in contiguous or non-contiguous memory. Moreover, a data structure, as used herein, does not require information to be co-located. It may be distributed across multiple servers, for example, that may be owned or operated by the same or different entities. Thus, for example, a data structure may include any data format that may be used to associate video footage with phase tags, event tags, and/or event characteristics.

FIG. 6 illustrates an example data structure 600 consistent with the disclosed embodiments. As shown in FIG. 6, data structure 600 may comprise a table including video footage 610 and video footage 620 pertaining to different surgical procedures. For example, video footage 610 may include footage of a laparoscopic cholecystectomy, while video footage 620 may include footage of a cataract surgery. Video footage 620 may be associated with footage location 621, which may correspond to a particular surgical phase of the cataract surgery. Phase tag 622 may identify the phase (in this instance a corneal incision) associated with footage location 621, as discussed above. Video footage 620 may also be associated with event tag 624, which may identify an intraoperative surgical event (in this instance an incision) within the surgical phase occurring at event location 623. Video footage 620 may further be associated with event characteristic 625, which may describe one or more characteristics of the intraoperative surgical event, such as surgeon skill level, as described in detail above. Each video footage identified in the data structure may be associated with more than one footage location, phase tag, event location, event tag and/or event characteristic. For example, video footage 610 may be associated with phase tags corresponding to more than one surgical phase (e.g., “Calot's triangle dissection” and “cutting of cystic duct”). Further, each surgical phase of a particular video footage may be associated with more than one event, and accordingly may be associated with more than one event location, event tag, and/or event characteristic. It is understood, however, that in some embodiments, a particular video footage may be associated with a single surgical phase and/or event. It is also understood that in some embodiments, an event may be associated with any number of event characteristics, including no event characteristics, a single event characteristic, two event characteristics, more than two event characteristics, and so forth. Some non-limiting examples of such event characteristics may include skill level associated with the event (such as minimal skill level required, skill level demonstrated, skill level of a medical care giver involved in the event, etc.), time associated with the event (such as start time, end time, etc.), type of the event, information related to medical instruments involved in the event, information related to anatomical structures involved in the event, information related to medical outcome associated with the event, one or more amounts (such as an amount of leak, amount of medication, amount of fluids, etc.), one or more dimensions (such as dimensions of anatomical structures, dimensions of incision, etc.), and so forth. Further, it is to be understood that data structure 600 is provided by way of example and various other data structures may be used.

Disclosed systems and methods may involve analyzing surgical footage to identify features of surgery, patient conditions, and surgical intraoperative events to obtain information for populating the operative report (a.k.a. postoperative report, post-operative report, surgical report, postsurgical report, or post-surgical report). An operative report may be populated by analyzing surgical data obtained from a surgical procedure to identify features of surgery, patient conditions, and surgical intraoperative event and extracting information from the analyzed data for populating the operative report. Therefore, there is a need for analyzing surgical data, and extracting information from the surgical data that may be used for populating an operative report. It is to be understood that any reference to an operative report is equally applicable to other types of documents, including but not limited to textual documents, multimedia documents, structured documents, or unstructured documents, and vice versa.

Aspects of this disclosure may relate to populating an operative report of a surgical procedure, including methods, systems, devices, and computer readable media. For ease of discussion, a method is described below, with the understanding that aspects of the method apply equally to systems, devices, and computer readable media. For example, some aspects of such a method may occur electronically over a network that is either wired, wireless, or both. Other aspects of such a method may occur using non-electronic means. In the broadest sense, the method is not limited to particular physical and/or electronic instrumentalities, but rather may be accomplished using many differing instrumentalities.

Consistent with disclosed embodiments, a method for populating an operative report of a surgical procedure may include receiving an input of an identifier of a patient. Further, the method may include receiving an input of an identifier of a health care provider. An operating report may be any suitable computer-based or paper-based report documenting a surgical procedure. In various embodiments, an operative report may include multiple frames of surgical footage, audio data, image data, text data (e.g., doctor notes) and the like. In an example embodiment, an operative report may be populated, partially populated, or not populated. For example, the operative report may contain fields (e.g., regions of the report) for holding various details obtained during the surgical procedure. In an example embodiment, at least some fields may have an associated characteristic (also referred to as a field name) that may determine what type of information can be entered in the field. For instance, a field with an associated name “Name of a Patient” may allow a name of a patient to be entered in that field. A field named “Pulse Plot” may be a field for displaying a pulse of a patient during the surgical procedure plotted as a function of time. In various embodiments, when the report is not populated, all the fields in the report may be empty; when the report is partially populated, some of the fields may contain information obtained from a surgical procedure; and when the report is fully populated (or mostly populated) the vast majority of the fields may contain information relating to an associated surgical procedure. In some examples, at least part of an operative report may have a free form format, allowing users and/or automatic processes to enter data in various organizations and/or formats, such as free text, which in some examples may include other elements embedded freely in the free text or accompanying it, such as links to external elements, images, videos, audio recordings, digital files, and so forth. It is appreciated that any detail described herein as included in an operative report in a particular field may be equally included in an operative report as part of such free textual information, embedded in the free text, or accompanying it.

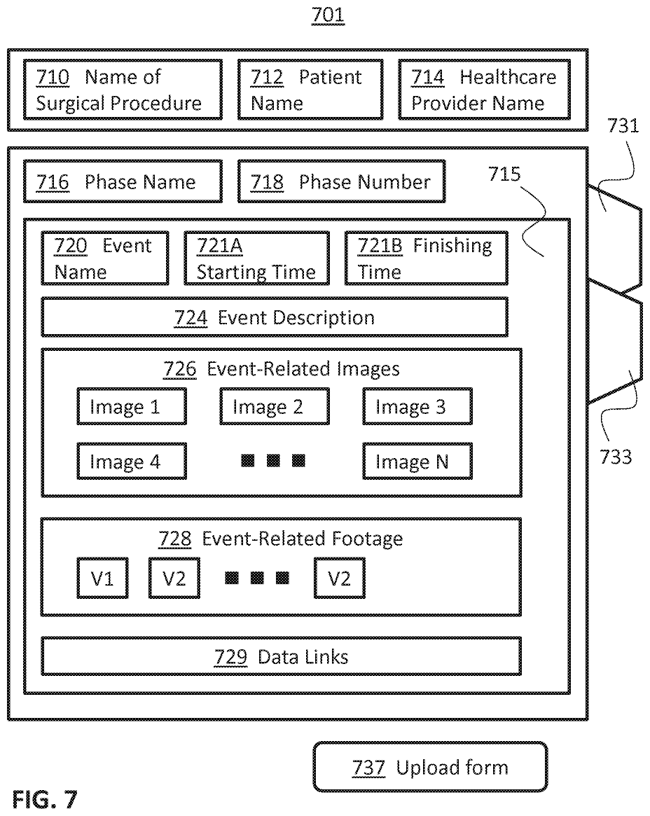

An example operative report 701 is shown in FIG. 7. Report 701 may contain multiple fields, sections, and subsections. Different fields may contain different types of information. For example, field 710 may contain a name of the surgical procedure, field 712 may contain a name of a patient and field 714 may contain a name of a healthcare provider. Field 716 may include a name of a phase of a surgical procedure, and field 718 may include a sequential number of a phase (e.g., a first phase of a surgical procedure). Multiple instances of fields 714 and/or 716 may be included in operative report 701, to describe a plurality of phases of the surgical procedure. Report 701 may include a section 715 that may describe a particular event during a surgical procedure. Multiple sections for describing multiple events may be present in report 701. One or more of the events may be connected to a particular surgical phase, while other events may not be connected to any surgical phase. In an example embodiment, section 715 may include a field 720 containing a name of the event, field 721A containing a starting time for the event, field 721B containing a finishing time for the event, and field 724 containing description of the event (e.g., field 724 may contain notes from a healthcare provider describing the event). Section 715 may include subsection 726 for containing fields for images such as fields IMAGE 1 through IMAGE N, as well as subsection 728 for containing event-related surgical footage. For example, subsection 728 may include fields V1-VN. Additionally, section 715 may include subsection 729 that may contain links to various other data related to a surgical procedure. In various embodiments, an operative report may be partitioned into different portions indicated by tabs 731 and 733, as shown in FIG. 7. For example, when a user selects tab 731, information related to a first portion of a surgical report may be displayed, and when a user selects tab 733, information related to a second portion of a surgical report may be displayed. In various embodiments, a surgical report may include any suitable number of portions.

FIG. 7 also shows that information may be uploaded into report 701, via an upload input form 737. For example, a user may click on a field (e.g., field V1, as shown in FIG. 7), and form 737 may be presented to the user for uploading data for the field V1. In various embodiments, fields, sections, subsections, and tabs, as shown in FIG. 7 are only illustrative, and any other suitable fields, sections, subsections, and tabs may be used. Furthermore, a number and types of fields, sections, subsections, and tabs may depend on information entered in operative report 701.

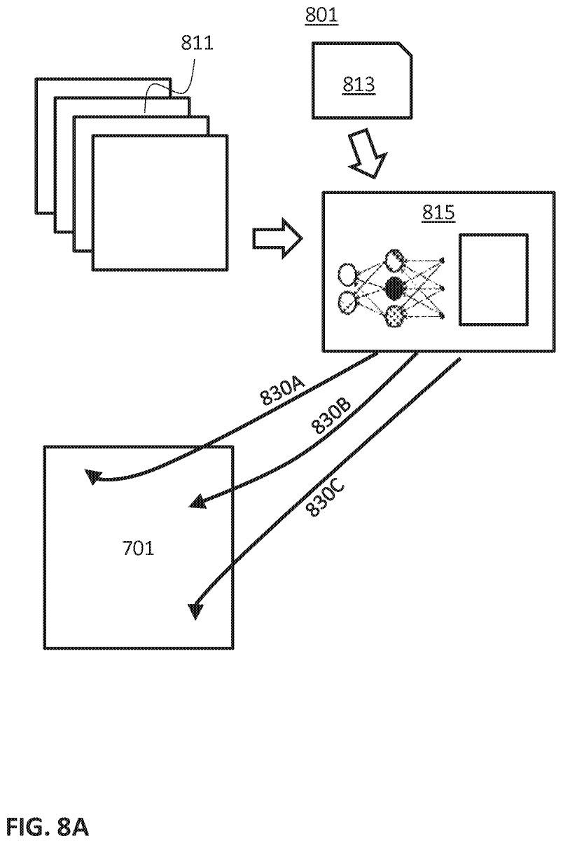

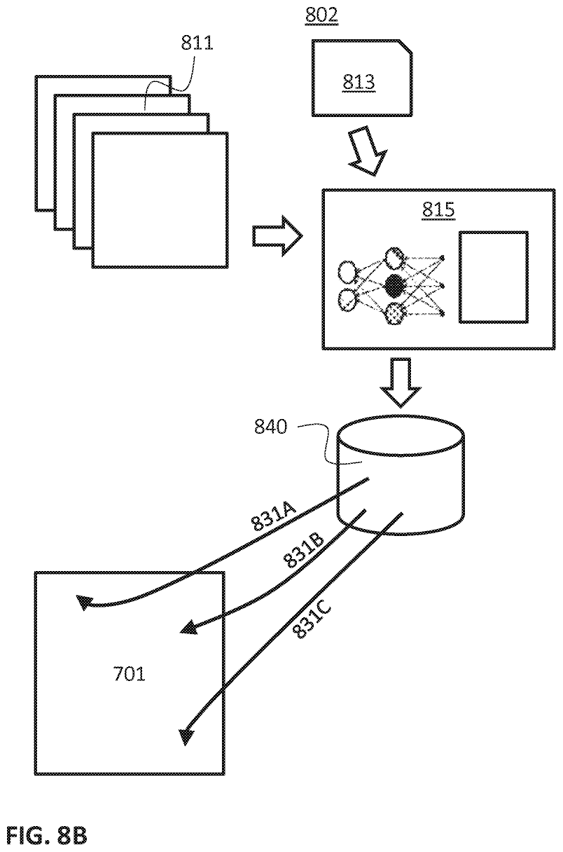

In various embodiments, information for populating at least part of an operative report may be obtained from surgical footage of a surgical procedure. Such information may be referred to as image-based information. Additionally, information about a surgical procedure may be obtained from notes of a healthcare provider or a user, previously filed forms for a patient (e.g., a medical history for the patient), medical devices used during a surgical procedure, and the like. Such information may be referred to as auxiliary information. In an example embodiment, auxiliary information may include vital signs, such as pulse, blood pressure, temperature, respiratory rate, oxygen levels, and the like reported by various medical devices used during a surgical procedure. Image-based information and auxiliary information may be processed by a suitable computer-based software application and the processed information may be used to populate an operative report. For example, FIG. 8A shows an example of a process 801 for processing information and populating an operative report 701. In an example embodiment, image-based information 811 and auxiliary information 813 may be used as an input to a computer-based software application 815, and application 815 may be configured to process information 811 and 813, extract data for various fields present in an operative report (e.g., report 701, as shown in FIG. 8A), and populate the various fields (as schematically indicated by arrows 830A-830C). FIG. 8B shows an example system 802 for processing information and populating an operative report 701. 802 may differ from system 801 in that various data processed by application 815 may be stored in a database 840 prior to populating operative report 701. By storing data in database 840, the data may be easily accessed for use in generating various other reports. Database 840 may be configured to execute a software application for mapping data from database 840 to fields of report 701 as schematically shown by arrows 831A-831C.

As described above, embodiments for populating an operative report may include receiving an input of an identifier of a patient and a healthcare provider. The identifier of a patient may be any suitable data or physical indicator (e.g., a patient's name, date of birth, social security number or other government identifier, patient number or other unique code, patient image, DNA sequence, a vocal ID, or any other indicator that uniquely identifies the patient. In some cases, a group of identifiers may be used as a combined identifier. In an example embodiment, an identifier may be an alphanumerical string that uniquely identifies the patient.

In various embodiments, of the patient identifier may be received as an input. This may occur using any suitable process of transmission (e.g., a process of transmission of data over a wired or wireless network, a process of transmission of data using a suitable input device such as a keyboard, mouse, joystick, and the like). In some cases, “receiving an input” may include receipt through mail or courier (e.g., a paper document delivered in person).

Similar to the patient identifier, the identifier of a health care provider may be any suitable indication of identity, such as a name, a code, an affiliation, an address, an employee number, a Physician License Number, or any other mechanism of identifying the healthcare provider. In an example embodiment, an identifier may be an alphanumerical string that uniquely identifies the healthcare provider.

Disclosed embodiments may further include receiving an input of surgical footage of a surgical procedure performed on the patient by the health care provider. Surgical footage may be received as input by a computer-based software application for analyzing the input (e.g., application 815, as shown in FIG. 8A) and/or, in some cases, receiving an input may include receiving the input by a healthcare professional or a user. This may occur, for example, when a healthcare professional or the user uploads the video footage from a storage location and/or directly from sensors capturing the video footage.

The surgical footage of a surgical procedure may include any form of recorded visual data, including recorded images and/or video data, which may also include sound data. Visual data may include a sequence of one or more images captured by image sensors, such as cameras 115, 121, 123, and/or 125, as described above in connection with FIG. 1. Some of the cameras (e.g., cameras 115, 121, and 125) may capture video/image data of operating table 141, and camera 121 may capture video/image data of a surgeon 131 performing the surgery. In some cases, cameras may capture video/image data associated with surgical team personnel, such as an anesthesiologist, nurses, surgical tech and the like located in operating room 101.

In various embodiments, image sensors may be configured to capture the surgical footage by converting visible light, x-ray light (e.g., via fluoroscopy), infrared light, or ultraviolet light to images, a sequence of images, videos, and the like. The image/video data may be stored as computer files using any suitable format such as JPEG, PNG, TIFF, Audio Video Interleave (AVI), Flash Video Format (FLV), QuickTime File Format (MOV), MPEG (MPG, MP4, M4P, etc.), Windows Media Video (WMV), Material Exchange Format (MXF), and the like.

A surgical procedure may include any medical procedure associated with or involving manual or operative procedures on a patient's body. Surgical procedures may include cutting, abrading, suturing, or other techniques that involve physically changing body tissues and/or organs. Surgical procedures may also include diagnosing patients or administering drugs to patients. Some examples of such surgical procedures may include a laparoscopic surgery, a thoracoscopic procedure, a bronchoscopic procedure, a microscopic procedure, an open surgery, a robotic surgery, an appendectomy, a carotid endarterectomy, a carpal tunnel release, a cataract surgery, a cesarean section, a cholecystectomy, a colectomy (such as a partial colectomy, a total colectomy, etc.), a coronary angioplasty, a coronary artery bypass, a debridement (for example of a wound, a burn, an infection, etc.), a free skin graft, a hemorrhoidectomy, a hip replacement, a hysterectomy, a hysteroscopy, an inguinal hernia repair, a knee arthroscopy, a knee replacement, a mastectomy (such as a partial mastectomy, a total mastectomy, a modified radical mastectomy, etc.), a prostate resection, a prostate removal, a shoulder arthroscopy, a spine surgery (such as a spinal fusion, a laminectomy, a foraminotomy, a diskectomy, a disk replacement, an interlaminar implant, etc.), a tonsillectomy, a cochlear implant procedure, brain tumor (for example meningioma, etc.) resection, interventional procedures such as percutaneous transluminal coronary angioplasty, transcatheter aortic valve replacement, minimally Invasive surgery for intracerebral hemorrhage evacuation, or any other medical procedure involving some form of incision. While the present disclosure is described in reference to surgical procedures, it is to be understood that it may also apply to other forms of medical procedures or procedures generally.

In various embodiments, the surgical procedure may be performed on the patient by a healthcare provider, with the patient being identified by the identifier, as described above. The healthcare provider may be a person, a group of people, an organization, or any entity authorized to provide health services to a patient. For example, the healthcare provider may be a surgeon, an anesthesiologist, a nurse practitioner, a general pediatrician, or any other person or a group of people that may be authorized and/or able to perform a surgical procedure. In various embodiments, the healthcare provider may be a surgical team for performing the surgical procedure and may include a head surgeon, an assistant surgeon, an anesthesiologist, a nurse, a technician, and the like. The healthcare provider may administer a surgical procedure, assist with the surgical procedure for a patient and the like. A hospital, clinic, or other organization or facility may also be characterized as a healthcare provider, consistent with disclosed embodiments. Likewise, a patient may be a person (or any living creature) on whom a surgical procedure is performed.

Aspects of disclosed embodiments may include analyzing a plurality of frames of the surgical footage to derive image-based information for populating an operative report of the surgical procedure. In various embodiments, image-based information may include information about events that occurred during the surgical procedure, information about phases of the surgical procedure, information about surgical tools used during the surgical procedure, information about anatomical structures on which the surgical procedure was performed, data from various devices (e.g., vital signs, such as pulse, blood pressure, temperature, respiratory rate, oxygen levels, and the like), or any other suitable information that may be obtained from the images and may be applicable to be documented in the operative report. Some other non-limiting examples of information based on an analysis of surgical footage and/or algorithms for analyzing the surgical footage and determining the information are described in this disclosure.

In various embodiments, the image-based information may be derived from the surgical footage using any suitable trained machine-learning model (or other image recognition algorithms) for identifying events, phases of surgical procedures, surgical tools, anatomical structures within the surgical footage, and the like, for example as described above. In some cases, the machine learning method may identify various properties of events, phases, surgical tools, anatomical structures, and the like. For example, a property of an event such as an incision may include the length of the incision, and a property of an anatomical structure may include a size of the structure or shape of the structure. In various embodiments, any suitable properties may be identified using a machine-learning method, for example as described above, and once identified may be used to populate a surgical report.

In various embodiments, the derived image-based information may be used for populating an operative report of the surgical procedure. A process of populating the operative report may include populating fields of the report with information specific to the fields. In an example embodiment, populating an operative report may be done by a computer-based application (e.g., application 815, as shown in 8A). For example, the computer-based application may be configured to retrieve a field from the operative report, determine a name associated with the field, determine what type of information (e.g., image-based information, or any other suitable information) needs to be entered in the field based on a determined name, and retrieve such information from either surgical footage or from auxiliary information (e.g., auxiliary information 813, as shown in FIG. 8A). In an example embodiment, retrieving information may include deriving image-based information from the surgical footage. For example, if the field name “Surgical Tools Used,” retrieving information may include using an image recognition algorithm for identifying (in the surgical footage) surgical tools used during the surgical procedure, and populating the surgical report with the names of the identified tools. Thus, derived image-based information may be used to populate the operative report of the surgical procedure. Other examples of image-based information that may be used to populate the report may include the starting and ending times of a procedure or portion thereof, complications encountered, conditions of organs, and other information that may be derived through analysis of video data. These might also include, characteristics of a patient, characteristics of one or more healthcare providers, information about an operating room (e.g., the type of devices present in the operating room, type of image sensors available in the operating room, etc.), or any other relevant data.

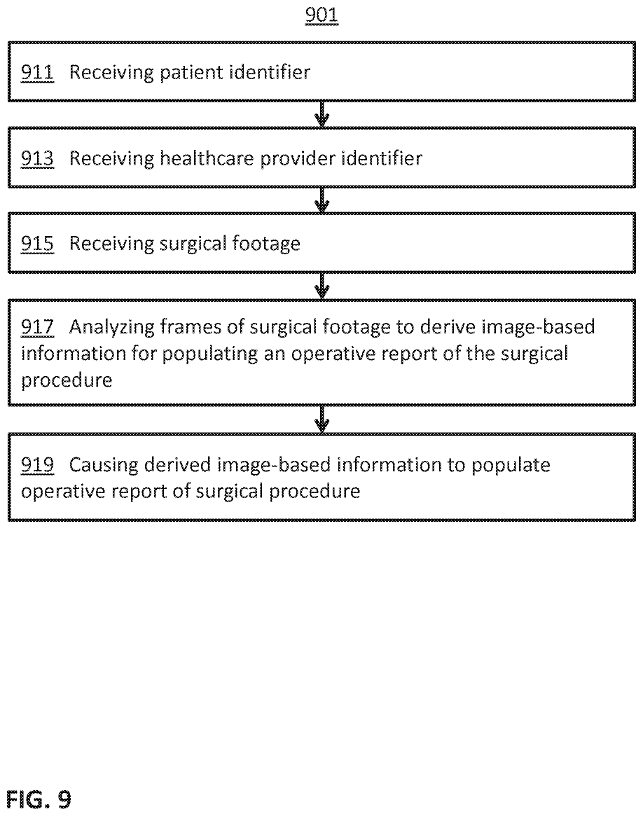

Aspects of a method of populating an operative report of a surgical procedure are illustrated by an example process 901, as shown in FIG. 9. At step 911 of process 901, the method may include receiving an input of an identifier of a patient, and at step 913, the method may include receiving an input of an identifier of a health care provider, as described above. At step 915, the method may include receiving an input of surgical footage of a surgical procedure performed on a patient by a health care provider. Receiving the input of surgical footage may include receiving the input by a suitable computer-based software application or a healthcare professional, as discussed above. At step 917, the method may include analyzing a plurality of frames of the surgical footage to derive image-based information for populating an operative report of the surgical procedure, as described herein, and at step 919, the method may include causing the derived image-based information to populate the operative report of the surgical procedure, as previously described.

Aspects of a method of populating an operative report of a surgical procedure may include analyzing the surgical footage to identify one or more phases of the surgical procedure. The phases may be distinguished from each other automatically based on a training model trained to distinguish one portion of a surgical procedure from another, for example as described herein.

For the purposes of the present disclosure, a phase may refer to a particular period or stage of a process or series of events. Accordingly, a surgical phase may refer to a sub-portion of a surgical procedure. For example, surgical phases of a laparoscopic cholecystectomy surgery may include trocar placement, preparation, calot's triangle dissection, clipping and cutting of cystic duct and artery, gallbladder dissection, gallbladder packaging, cleaning and coagulation of liver bed, gallbladder retraction, and so forth. In another example, surgical phases of a cataract surgery may include preparation, povidone-iodine injection, corneal incision, capsulorhexis, phaco-emulsification, cortical aspiration, intraocularlens implantation, intraocular-lens adjustment, wound sealing, and so forth. In yet another example, surgical phases of a pituitary surgery may include preparation, nasal incision, nose retractor installation, access to the tumor, tumor removal, column of nose replacement, suturing, nose compress installation, and so forth. Some other examples of surgical phases may include preparation, incision, laparoscope positioning, suturing, and so forth.

In some examples, the user may identify a phase by marking a section of the surgical footage with a word/sentence/string that identifies a name or a type of a phase. The user may also identify an event, procedure, or device used, which input may be associated with particular video footage (e.g., for example through a lookup table or other data structure). The user input may be received through a user interface of a user device, such as a desktop computer, a laptop, a tablet, a mobile phone, a wearable device, an internet of things (IoT) device, or any other means for receiving input from a user. The interface may provide, for example, one or more drop-down menus with one or more pick lists of phase names; a data entry field that permits the user to enter the phase name and/or that suggests phase names once a few letters are entered; a pick list from which phase names may be chosen; a group of selectable icons each associated with a differing phase, or any other mechanism that allows users to identify or select a phase.

In some embodiments, analyzing the surgical procedure to identify one or more phases of the surgical procedure may involve using computer analysis (e.g., a machine-learning model) to analyze frames of the video footage, for example as described above. Computer analysis may include any form of electronic analysis using a computing device. In some embodiments, computer analysis may include using one or more image recognition algorithms to identify features of one or more frames of the video footage. Computer analysis may be performed on individual frames or may be performed across multiple frames, for example, to detect motion or other changes between frames.

In some embodiments, analyzing the surgical procedure to identify at least one phase of the surgical procedure may involve associating a name with at least one phase. For example, if the identified phase includes gallbladder dissection, a name “gallbladder dissection” may be associated with that phase. In various embodiments, derived image-based information (derived from surgical footage of a surgical procedure by identifying a phase), may include an associated phase name, as described above.

Further, aspects of a method of populating an operative report of a surgical procedure may include identifying a property of at least one phase of identified phases. A property of a phase may be any characteristics of a phase such as a duration of the phase, a place of the phase in a sequence of phases during the surgical procedure, a phase complexity, an identification of a technique used, information related to medical instruments used in the phase, information related to actions performed in the phase, changes in a condition of an anatomical structure during the phase, or any other information that may characterize the phase. A phase property may be expressed in the form of an alphanumerical string. For instance, “a first phase” may identify the phase as a first phase in a sequence of phases during a surgical procedure, “one hour” may describe that the phase has a duration of one hour, “bronchoscopy” may identify a phase as a bronchoscopy, and the like. Additionally or alternatively, a property of a phase may be non-textural data (e.g., image, audio, numerical, and/or video data) collected during a surgical procedure. For example, a representative image of an anatomical structure (or surgical instrument, or an interaction of a surgical instrument with an example anatomical structure) performed during a phase of a surgical procedure may be used as a property of a phase. In one example, a machine learning model may be trained using training examples to identify properties of surgical phases from images and/or videos. An example of such training example may include an image and/or a video of at least a portion of a surgical phase of a surgical procedure, together with a label indicating one or more properties of the surgical phase. Some non-limiting examples of such properties may include a name of the surgical phase, a textual description of the surgical phase, or any other property of a surgical phase described above. Further, in some examples, the trained machine learning model may be used to analyze the surgical footage to identify the property of the at least one phase of identified phases. In various embodiments, the derived image-based information (used for populating the surgical record) may be based on the identified at least one phase and the identified property of the at least one phase. For example, the combination of both the phase and the property together may enable the phase to be recorded in a way that is more meaningful. For example, during a phase of suturing of a valve, if an intraoperative leak is detected (a property of the phase), the phase/property combination may be recorded in the surgical record. In some cases, the derived image-based information may include a segment of a video captured during the phase of the surgical procedure.

Aspects of a method of populating an operative report of a surgical procedure may include determining at least a beginning of the at least one phase; and wherein the derived image-based information is based on the determined beginning. The beginning of at least one phase may be determined by performing a computer image analysis on surgical footage, for example as described above. For example, using a trained machine learning model (such as a recurrent convolutional neural network), the beginning of a particular phase may be distinguished from the end of a prior phase, and the location may be identified and stored in the surgical record. In another example, a phase may start when a particular medical instrument first appears in the video footage, and an object detection algorithm may be used to identify the first appearance of the particular medical instrument in the surgical footage.

In some cases, a time marker may be associated with the at least one phase, and the derived image-based information may include the time marker associated with the at least one phase. The time marker may be recorded in a number of ways, including, a time elapsed from the beginning of the surgical procedure, the time as measured by the time of day, or a time as it relates to some other intraoperative recorded time. In various embodiments, a time marker may be associated with the beginning of each identified phase (e.g., a time marker may be associated with the beginning location of the surgical phase within the surgical footage). The time marker may be any suitable alphanumerical identifier, or any other data identifier (e.g., an audio signal or an image) and may include information about a time (and/or possibly a time range), associated with the beginning of the identified phase.

An example surgical event, such as an incision, may be detected using action detection algorithms, for example as discussed above. Such an identified surgical event may identify a beginning of a surgical phase. In an example embodiment, an event that begins a surgical phase may be detected based on machine learning techniques. For example, a machine learning model may be trained using historical surgical footage including known events that begin the surgical phase.

Further, disclosed embodiments may include determining at least an ending of the at least one phase, and derived image-based information may be based on the determined ending. The end of the surgical phase may be determined by detecting an end location of the surgical phase within the surgical footage. In various embodiments, a time marker may be associated with the end of each identified phase (e.g., the time marker may be associated with the end location of the surgical phase within the surgical footage). As discussed above, the ending marker may be recorded in the same manner as the starting marker, and may be characterized by any suitable alphanumerical identifier, or any other data identifier. For example, the surgical footage may be analyzed to identify the beginning of a successive surgical phase, and the ending of one phase may be identical to the beginning of the successive surgical phase. In another example, a phase may end when a particular medical instrument last appears in the video footage, and an object detection algorithm may be used to identify the last appearance of the particular medical instrument in the surgical footage.

Embodiments for automatically populating an operative report of a surgical procedure may also include transmitting data to a health care provider, the transmitted data, including a patient identifier and derived image-based information. During or after a surgical procedure, video captured during the surgical procedure may be transmitted to a healthcare provider for populating the patient's associated surgical record. In order to ensure that the video populates the appropriate record, the patient identifier may accompany the video in the transmission. In some embodiments, this may enable the surgical record to be automatically updated with the video, without human intervention. In other embodiments, either on the transmission and/or the receiving end, a human may select the video for transmission, or accept the video for incorporation into the patient's medical record. In some cases, transmitting data may involve mailing (or delivering in person) a physical copy (e.g., a paper copy, a CD-ROM, a hard drive, a DVD, a USB drive, and the like) of documents describing the data. Additionally or alternatively, transmitting data may include transmitting data to at least one of a health insurance provider or a medical malpractice carrier.

Aspects of the disclosure may include analyzing the surgical footage to identify at least one recommendation for post-operative treatment; and providing the identified at least one recommendation. As described earlier, surgical footage may be analyzed in various ways (e.g., using a machine-learning method, by a healthcare provider, and the like). In various embodiments, a machine-learning method may be configured not only to recognize events within the video frames but also configured to form conclusions about various aspects of the surgical procedure based on an analysis of surgical footage. For example, post-operative wound care may vary depending on the nature of the surgical wound. Video analysis might determine that nature, and might also provide a recommendation for post-operative treatment of the wound site. Such information may be transmitted to and stored in the surgical record. In some cases, the machine-learning method may identify intraoperative events (e.g., adverse events) and may provide indications for these events for which specific post-operative treatments are needed. This may be analyzed through machine learning and the recommendation for post-operative treatment may be automatically provided. In one example, in response to a first surgical event identified in the surgical footage, a first recommendation for post-operative treatment may be identified, and in response to a second event identified in the surgical footage, a second recommendation for post-operative treatment may be identified, the second recommendation may differ from the first recommendation. In one example, in response to a first condition of an anatomical structure identified in the surgical footage, a first recommendation for post-operative treatment may be identified, and in response to a second condition of the anatomical structure identified in the surgical footage, a second recommendation for post-operative treatment may be identified, the second recommendation may differ from the first recommendation. In some examples, a machine learning model may be trained using training examples to generate recommendations for post-operative treatment from surgical images and/or surgical videos, and the trained machine learning model may be used to analyze the surgical footage and identifying the at least one recommendation for post-operative treatment. An example of such training example may include an image or a video of at least a portion of a surgical procedure, together with a label indicating the desired recommendations for post-operative treatment corresponding to the surgical procedure.

Such recommendations may include suggesting physical therapy, medications further physical examination, a follow on surgical procedure, and the like. In some cases, recommendations may not directly relate to medical activities but may include diet recommendations, sleep recommendations, recommendations for physical activity, or recommendations for stress management. In various embodiments, the identified recommendation may be provided to a healthcare professional responsible for a post-operative treatment for the patient. Additionally or alternatively, the recommendation may be provided to a third party which may be a patient, a family member, a friend, and the like.

In one embodiment, an analysis of surgical footage may include identifying that during a given time of a surgical procedure, a surgeon may have worked too closely to intestines of a patient, for example, using an energy device. When such an event is identified (for example using an object detection algorithm, using a trained machine learning model, etc.), a notification (e.g., a push notification) may be send to alert a surgeon (or any other healthcare professional supervising a post-operative treatment of a patient) to further analyze the surgical footage and to have special procedures planned to avoid a catastrophic post-operative event (e.g., bleeding, cardiac arrest, and the like).

In various embodiments, populating an operative report of a surgical procedure may include enabling a health care provider to alter at least part of derived image-based information in the operative report. For example, the healthcare provider (also referred to as a healthcare professional) may access an operative report via a software application configured to display information in the operative report. In various embodiments, a healthcare professional may be enabled to alter some or all fields within the operative report. In some embodiments, particular fields may be locked as unalterable without administrative rights. Examples of alterable fields may be those containing text-based data (e.g., alterable by inputting new data via keyboard, mouse, microphone, and the like), image data (e.g., by uploading one or more images related to a surgical procedure, overlaying information over the one or more images, etc.), video data (e.g., by uploading one or more videos related to a surgical procedure overlaying information over one or more frames of the one or more videos, etc.), audio data (e.g., the audio data captured during a surgical procedure), and the like.

In various embodiments, updates to an operative report may be tracked using a version tracking system. In an example embodiment, the version tracking system may maintain all data that was previously used to populate an operative report. The version tracking system for may be configured to track differences between different versions of an operative report, and may be configured to track information about a party (e.g., a name of a healthcare professional, a time of the update, and the like) that made changes to the report.

In some embodiments, populating an operative report of a surgical procedure may be configured to cause at least part of derived image-based information to be identified in an operative report as automatically generated data. In various embodiments, as derived image-based information is used to populate an operative report, populating the report may include identifying how the derived image-based information was generated. For example, if an elevated heart rate was determined using computer vision analysis of detected pulses in vascular, the source of that determination might be noted as being based on a video determination. Similarly, video analysis might automatically estimate a volume of blood loss as the result of a rupture, and the surgical report might note, along with the estimated loss, that the volume of loss is an estimation based on video analysis. Indeed, any indication derived from video analysis might be so noted in the operative report using any textual, graphical, or icon based information to reflect the source of the data. For example, a movie icon may appear next to data derived from video. Alternatively, if a healthcare professional identifies an event within surgical footage and provides a segment of surgical footage corresponding to the identified event as a derived image-based information, such information may be considered as generated by the healthcare professional and may not be classified as automatically generated data.

Disclosed embodiments may include analyzing surgical footage to identify a surgical event within the surgical footage, for example as described above. The analysis, as previously discussed, may occur using a machine learning model. The identification may be derived from historical data where surgical events were already identified, along with a name for the event. Thus, when a similar even is detected through machine learning, the previously identified name for that event can similarly be applied to a current event identification.

Further, consistent with disclosed embodiments, not only may an event be identified, but also a property of a surgical event may also be identified. The property of a surgical event may be a type of an event or any other information characterizing the event. For example, if the event is an incision, the machine-learning model may be configured to return a name “incision” as a type of the event, and a length and a depth of the incision as a property of the event. In some cases, a predetermined list of possible types for various events may be provided to a machine-learning model, and the machine-learning model may be configured to select a type from the list of event types to accurately characterize an event. The number of properties can vary based on the type of event identified. Some rather straightforward events may have a relatively short list of associated properties, while other events may have many more associated alternative properties.

As discussed, machine-learning models are one way for identifying events, with the models trained using examples to identify (or determine) events. The training may involve any suitable approach, such as for example, a supervised learning approach. For instance, historical surgical footage containing features corresponding to an event may be presented as input data for the machine-learning model, and the machine-learning model may output the name of the event corresponding to the features within the footage. Various parameters of the machine-learning model may be adjusted to train the machine-learning model to correctly identify events corresponding to the features within the historical visual data. For example, if the machine-learning model is a neural network, parameters of such a neural network (e.g., weights of the network, number of neurons, activation functions, biases of the network, number of layers within the network, and the like) may be adjusted using any suitable approach (e.g., weights of the neural network may be adjusted using a backpropagation process).

In one embodiment, the event may be identified by a medical professional (e.g., a surgeon), and the event may be tagged at the time of its occurrence. If a machine learning model identifies surgical activity as potentially of interest but lacks an associated name for the activity, the associated footage may be saved and a user might later be prompted to provide an associated name.

In some cases, a surgeon may mark an event during a surgical procedure for subsequent identification. For example, the surgeon may mark the event using a visual or an audio signal (e.g., a hand gesture, a body gesture, a visual signal produced by a light source generated by a medical instrument, a spoken word, and the like) that may be captured by one or more image sensors/audio sensors and recognized as a trigger for an event.

In various embodiments, derived image-based information may be based on an identified surgical event and an identified property of the event. After an event and one or more properties of the event are identified as discussed earlier, the combination of can be analyzed to determine image-based information that may not have been derivable from either the event or the property alone. For example, if a particular property of a particular event is associated with a known risk of post-operative complication, that risk may be determined and included in the image-based information. Alternatively, by way of example, the derived image-based information may include one or more of a name of the event, a segment of a surgical footage corresponding to the event, a name and/or image of a surgical instrument used during the event, a name and/or image of an anatomical structure operated during the event, an image of interaction of the surgical instrument and the anatomical structure, a duration time for the event, and/or any other information derived from the video.

As mentioned, the surgical footage may be analyzed to determine an event name of the identified surgical event. As described above, the event name may be determined using a suitable machine-learning model. Alternatively, a name of the event may be identified by a healthcare professional. In various embodiments, the derived image-based information may include the determined event name.

Aspects of disclosed embodiments may also include associating a time marker with an identified surgical event. A process of associating a time marker with an identified surgical event may be similar to the process of associating a time marker with a phase of a surgical procedure. For example, a time marker may be associated with a beginning of an event of a surgical procedure (e.g., the beginning or some other intermediate location or range of locations of a surgical event within surgical footage). A time marker may be any suitable alphanumerical identifier, or any other graphical or data identifier. For example, the time marker may be an icon or other graphic that appears on an active or static timeline of some or all of a surgical procedure. If active, the time marker may be clickable (or otherwise selectable) to cause footage of the associated event to be presented. The marker may be caused to appear in footage, either through a textual or graphic overlay on the footage or through an identifying audio indicator embedded for playback presentation. Such indicators may include one or more pieces of information such as temporal data (time or time range of the occurrence), location data (wherein the event occurred), or characterizing data (describing properties of the occurrence.) In some situations, a time marker may be associated with an end of an event (e.g., the time marker may be associated with an end location of the event within the surgical footage). Derived image-based information may include multiple time markers, for multiple events and/or for multiple locations within events.

In some embodiments, providing the derived image-based information may occur in a form that enables updating an electronic medical record. For example, derived image-based information may include text data, image data, video data, audio data, and the like, that may be in a form that can be uploaded to a software application that may store and display an electronic medical record (e.g., a standalone application for storing and displaying a medical record, a web-interface for displaying a medical record using information stored in a database, and the like). In various embodiments, the software application for storing and displaying a medical record may include an interface for updating the electronic medical record using derived image-based information. The interface may include graphical user elements for uploading image, video and audio data, for uploading text data, for typing text data into the electronic medical record, for updating the electronic medical record using a computer mouse, and the like.

In various embodiments, the derived image-based information may be based in part on a user input. For example, a user, such as a healthcare professional, may provide inputs while the surgical footage is being captured, for example as described above, and the derived image-based information may be partly based on such inputs. For example, such input may indicate a particular point in time within the surgical footage.

In various embodiments, the derived image-based information may include a first part associated with a first portion of a surgical procedure and a second part associated with a second portion of a surgical procedure. Separating image-based information into parts may facilitate classifying the image-based information. For example, if the first portion of the surgical procedure involves making multiple incisions and a second portion of the surgical procedure involves suturing, such portions may be used to classify those portions of the surgical procedure. In some cases, during a first portion of a surgical procedure, a first set of sensors may be used to collect image-based information, and during a second portion of the surgical procedure, a different set of sensors may be used to collect image-based information. For example, during the first portion, image sensors located on a surgical instrument may be used to capture surgical footage, and during the second portion of the surgical procedure, overhead image sensors (i.e., image sensors located above an operating table) may be used to capture the surgical footage.

In various embodiments, the operative report may include a first portion corresponding to the first portion of the surgical procedure and a second portion corresponding to the second portion of the surgical procedure. The start of the first portion of the operative report may be indicated by a first position (e.g., the first position may be a pointer in a data file, a location of a cursor in a text file, a data record in a database, and the like). The start of the second portion of the operative report may be indicated by a second position, which may be any suitable indication of location in the file that is a starting point of the second portion of the operative report (e.g., the first position may be a pointer in a data file, a location of a cursor in a text file, a data record in a database, and the like). In various embodiments, an operative report may be separated into portions based on corresponding portions of a surgical procedure. In an example embodiment, a machine-learning method (or a healthcare provider) may identify portions of the surgical procedure and configure the operative report to have such identified portions. The operative report may not be limited to two portions but may include more or less than two portions.

Aspects of disclosed embodiments may include receiving a preliminary operative report. The operative report may be received by any entity, whether an organization, individual, or a computer (e.g., an insurance company or healthcare organization, a healthcare professional, or a computer-based program for populating operative reports, such as application 815, as shown in FIG. 8A). In various embodiments, analyzing a preliminary operative report may involve selecting a first position and a second position within the preliminary operative report, the first position is associated with a first portion of the surgical procedure and the second position is associated with a second portion of the surgical procedure. Such selection may enable someone (or a machine) analyzing the report to skip directly to an area of interest in the report. Thus, analyzing a preliminary operative report may include identifying indicators for one or more of a first position and a second position. The indicators may be any suitable alphanumeric or graphical indicators. For example, an indicator for the first position may be a text string “this is a start of the first portion of the operative report” or a graphical start icon. In one example, Natural Language Processing (NLP) algorithms may be used to analyze textual information included in the preliminary operative report, to identify in the textual information portions that discuss different aspects of the surgical procedure (such as different surgical phases, different surgical events, usage of different medical instruments, and so forth), and associate the identified portions of the textual information with different portions of the surgical procedure (for example, with the corresponding surgical phase, with the corresponding surgical events, with the usage of the corresponding medical instruments, and so forth). Further, in some examples, the first position and the second position (as well as additional positions) within the preliminary operative report may be based on and/or linked with the identified portions of the textual information.

Further, embodiments may include causing a first part of derived image-based information to be inserted at a selected first position and a second part of the derived image-based information to be inserted at a selected second position. For example, a first portion of an operative report may include a first set of fields that may be populated by derived image-based information captured during a first portion of the surgical procedure, and a second portion of the operative report may include a second set of fields that may be populated by derived image-based information captured during a second portion of the surgical procedure. In another example, a first part of derived image-based information may correspond to a first portion of the surgical procedure and a second part of derived image-based information may correspond to a second portion of the surgical procedure, the first position within the preliminary operative report may be identified as corresponding to the first portion of the surgical procedure (as described above), the second position within the preliminary operative report may be identified as corresponding to the second portion of the surgical procedure (as described above), and in response, the first part of derived image-based information may be inserted at the first position and the second part of the derived image-based information may be inserted at the second position. Some non-limiting examples of the first and second portions of the surgical procedure may include different surgical phases, different surgical events, usage of different medical instruments, different actions, and so forth.

Aspects of the present disclosure may also include analyzing surgical footage to select at least part of at least one frame of the surgical footage; and causing the selected at least part of at least one frame of the surgical footage to be included in an operative report of a surgical procedure. For example, if an operative report includes a field configured to hold one or more images of a surgical instrument used during a surgical procedure, an example machine-learning model may be configured to identify one or more frames of the surgical footage and select parts of the identified frames that contain a surgical instrument. Further, the selected part (or parts) of at least one frame may be inserted (e.g. populate) into the operative report. The machine-learning model may also be configured to extract other relevant frames of surgical footage. For example, frames of the surgical footage depicting an anatomical structure that is the focus of an operation, or frames depicting an interaction between a surgical instrument and an anatomical structure may be extracted. Such relevant frames may also populate the operative report.

Disclosed embodiments may also include receiving a preliminary operative report and analyzing the preliminary operative report and surgical footage to select the at least part of at least one frame of the surgical footage. For example, a machine-learning model may be configured to analyze an operative report and identify a discussion of an adverse event (e.g., bleeding). The adverse event may be identified, for example, through an indication stored in the operative report, using an NLP algorithm, and so forth. The indication may, for example, be an indication of a name of the adverse event. It may include a time when the adverse event occurred during a surgical procedure. The adverse event may be determined using a machine-learning model configured to retrieve surgical footage for the surgical procedure and identify a portion of a frame that shows a visual data representing the adverse event (e.g., a portion of a frame that shows bleeding). Further, in some examples, the identify portion of the frame may be inserted to the operative report in connection with the discussion of the adverse event, or be associated with the discussion of the adverse event in another way.

Additional aspects of disclosed embodiments may include analyzing the preliminary operative report and surgical footage to identify at least one inconsistency between the preliminary operative report and the surgical footage. In various embodiments, inconsistency may be determined by comparing information stored in the report with information derived through a machine learning model that determines an error. For illustrative purposes, one of a virtual infinite number of potential inconsistencies could occur when a medical professional indicates in the report that the surgical site was closed with sutures, while the video reveals that the site was closed with staples. The video revelation might occur, for example, with a computer-based software application (e.g., application 815, as shown in FIG. 8A) where an operative report is compared with video footage of the associated procedure. If a difference is noted, a computer-based software application may determine the source of the error, may note the error, may send a notification of the error, and/or may automatically correct the error. For example, the application may analyze various versions of a preliminary operative report (using, for example, a version tracking system, as described above) to identify at which step of generating the preliminary operative report the difference first appeared.

As previously mentioned, embodiments of the disclosure may include providing an indication of the identified at least one inconsistency. The indication may be provided by transmitting a notification to a healthcare professional using any suitable means, as discussed above.

Various embodiments may include receiving an input of a patient identifier and an input of an identifier of a health care provider, as previously described. Further, the method may include receiving an input of surgical footage of a surgical procedure performed on the patient by the health care provider, as previously described. The method may also include analyzing a plurality of frames of the surgical footage to identify phases of the surgical procedure based on detected interactions between medical instruments and biological structures and, based on the interactions, associate a name with each identified phase. For example, at least some of the frames of the surgical footage may indicate a portion of the surgical footage in which a surgical operation is being performed on a biological structure (herein, also referred to as an anatomical structure). As discussed above, the interaction may include any action by the medical instrument that may influence the biological structure or vice versa. For example, the interaction may include a contact between the medical instrument and the biological structure, an action by the medical instrument on the biological structure (such as cutting, clamping, grasping, applying pressure, scraping, etc.), a physiological response by the biological structure, the medical instrument emitting light towards the biological structure (e.g., surgical tool may be a laser that emits light towards the biological structure) a sound emitted towards anatomical structure, an electromagnetic field created in a proximity of the biological structure, a current induced into the biological structure, or any other suitable forms of interaction.

In some cases, detecting an interaction may include identifying proximity of the medical instrument to a biological structure. For example, by analyzing the surgical video footage, an image recognition model may be configured to determine a distance between the medical instrument and a point (or a set of points) on a biological structure.

Aspects of the present disclosure may involve associating a name with each identified phase based on detected interactions between medical instruments and biological structures. The name may be associated with each identified phase using any suitable means. For example, as described above, the name may be supplied by a user or may be automatically determined using a suitable machine learning method, as described above. In particular, a process of identifying a phase of a surgical procedure involves associating a name with each identified phase. In various embodiments, the name associated with the phase may include a name for a biological structure and a name of a surgical instrument interacting with the structure.

In various embodiments, the name associated with the identified phase may be updated, modified, quantified, or otherwise altered during the ongoing surgical phase or after the completion of the surgical phase. For example, a machine learning model may initially determine a name for the surgical phase as “incision” and may later update the name of the surgical phase, based on detected interactions between medical instruments and biological structures, to an illustrative name “a Lanz incision extending medially towards rectus abdominis, made via laparoscopic surgery using laparoscopic scissors.” Additionally or alternatively, a separate record (herein also referred to as a note) may be added to the name identifying the surgical phase, with the note containing various details and/or characteristics of the surgical phase. Such details may include an instrument used during the surgical phase, a light used during the surgical phase, a pressure value for the pressure applied on an example biological structure, an area over which the pressure was applied, one or more images of the biological structure and/or medical instrument during the surgical phase, identifications for events (e.g., adverse events such as bleeding), or any other related information characterizing the surgical phase.

Aspects of the present disclosure may also involve transmitting data to a health care provider, the transmitted data including the patient identifier, the names of the identified phases of the surgical procedure, and time markers associated with the identified phases.

An embodiment may include determining at least a beginning of each identified phase, and associating a time marker with the beginning of each identified phase, as discussed above. Additionally or alternatively, the time marker may identify an end of the identified phase, as discussed above. The transmitted data may include text, graphics, video data, animations, audio data, and the like. In some cases, the transmitted data may be an SMS message, an email, and the like delivered to any suitable devices (e.g., smartphones, laptops, desktops, TVs, etc.) in possession of various health care providers (e.g., various medical personnel, administrators, and other interested individuals or systems). In some cases, the transmitted data may also be provided to patients, relatives or friends of patients.

Further, aspects of the present disclosure may include populating an operative report with transmitted data in a manner that enables the health care provider to alter phase names in an operative report. Such alterations may occur through an interface that enables operative report alterations. For example, the interface may allow a healthcare provider to update the phase names by typing new phase names using a keyboard. In various embodiments, the interface may be also configured for altering names of various events identified in surgical footage and recorded in an operative report.

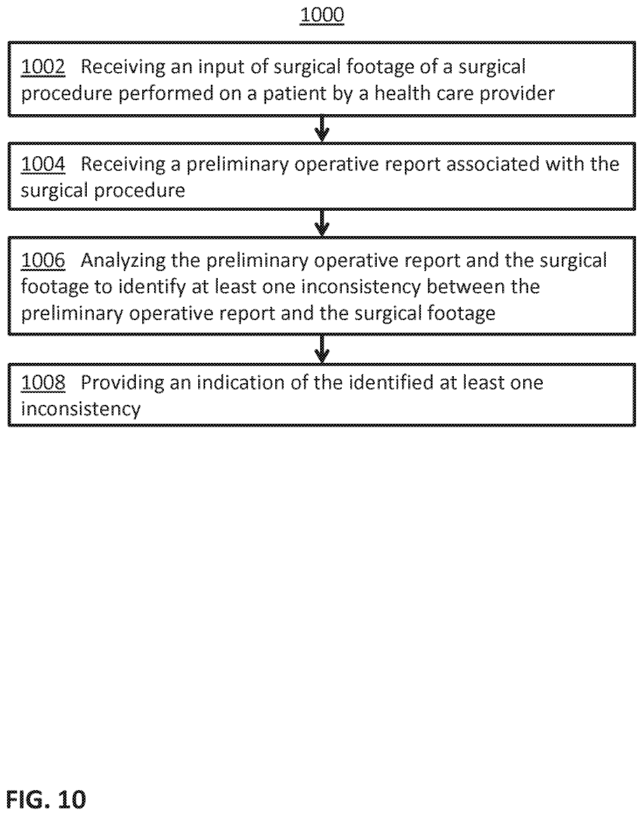

FIG. 10 is a flow diagram of an exemplary process 1000, consistent with some embodiments of the present disclosure. In this example, process 1000 may comprise receiving an input of surgical footage of a surgical procedure performed on a patient by a health care provider (step 1002); receiving a preliminary operative report associated with the surgical procedure (step 1004); analyzing the preliminary operative report and the surgical footage to identify at least one inconsistency between the preliminary operative report and the surgical footage (step 1006); and providing an indication of the identified at least one inconsistency (step 1008). In other examples, process 1000 may include additional steps or fewer steps. For example, process 1000 may further comprise step 1102 and/or step 1104. In other examples, one or more steps of process 1000 may be executed in a different order and/or one or more groups of steps may be executed simultaneously.

In some examples, a system may include at least one processing unit configured to perform process 1000 and/or one or more of the steps of process 1000. In one example, the system may further comprise a digital communication device configured to receive an input of surgical footage of a surgical procedure (such as the surgical footage of step 1002, a surgical footage of a surgical procedure performed on a patient by a health care provider, a surgical footage of a different surgical procedure, and so forth), and/or to receive a preliminary operative report associated with a surgical procedure (such as the preliminary operative report of step 1004, a different preliminary operative report, and so forth). In one example, the system may further comprise at least one image sensor configured to capture surgical footage of a surgical procedure (such as the surgical footage of step 1002, a surgical footage of a surgical procedure performed on a patient by a health care provider, a surgical footage of a different surgical procedure, and so forth). In one example, the system may further comprise at least one digital memory unit configured to store surgical footage of a surgical procedure (such as the surgical footage of step 1002, a surgical footage of a surgical procedure performed on a patient by a health care provider, a surgical footage of a different surgical procedure, and so forth), and/or a preliminary operative report associated with a surgical procedure (such as the preliminary operative report of step 1004, a different preliminary operative report, and so forth). Further, the at least one processing unit may be configured to read from the at least one digital memory unit the surgical footage and/or the preliminary operative report. In some examples, a non-transitory computer readable medium may store computer implementable instructions that when executed by at least one processor may cause the at least one processor to perform operations, and the operations may include process 1000 and/or one or more of the steps of process 1000. In some examples, a method may include performing the steps of process 1000.

In some examples, step 1002 may comprise receiving an input of surgical footage of a surgical procedure performed on a patient (for example, on the patient of step 911, on the patient of step 1102, on a different patient, and so forth) by a health care provider (for example, by the health care provider of step 913, by the health care provider of step 1104, by a different health care provider, and so forth). For example, step 1002 may use step 915 and/or step 510 to receive the input of the surgical footage. In another example, step 1002 may read the surgical footage from a digital memory unit, may receive the surgical footage from an external computing device (for example, using a digital communication device), may capture the surgical footage (for example, using a camera, using an image sensor, using an intracorporeal camera, etc.), may capture the image data from a physical environment, and so forth.

In some examples, step 1004 may comprise receiving a preliminary operative report associated with a surgical procedure (for example, a preliminary operative report associated with the surgical procedure of step 1002, a preliminary operative report associated with a different surgical procedure, and so forth), for example as described above. In one example, the preliminary operative report may be a preliminary operative report created manually (for example, by the health care provider of step 1002, by a different person, etc.), may be a preliminary operative report generated automatically (for example, as described above, based on an analysis of the surgical footage received by step 1002, using process 901, using process 1100, using process 1300, etc.), and so forth. One non-limiting example of such preliminary operative report is operative report 701. In one example, step 1004 may read the preliminary operative report from a digital memory unit, may receive the preliminary operative report from an external computing device (for example, using a digital communication device), may generate the preliminary operative report automatically, and so forth.

In some examples, step 1006 may comprise analyzing a preliminary operative report (such as the preliminary operative report received by step 1004, a different preliminary operative report, etc.) and surgical footage (such as the surgical footage received by step 1002, a different surgical footage, etc.) to identify at least one inconsistency between the preliminary operative report and the surgical footage, for example as described above. In some examples, step 1006 may use a trained machine learning model to analyze the preliminary operative report and the surgical footage to identify the at least one inconsistency between the preliminary operative report and the surgical footage. The trained machine learning model may be a machine learning model trained using training examples to analyze operative reports and/or surgical footages and/or additional information to identify inconsistencies between operative reports and surgical footages. An example of such training example may include a sample operative report, a sample surgical footage and sample additional information, together with a label indicating one or more inconsistencies between the sample operative report and the sample surgical footage. In some examples, step 1006 may use an artificial neural network to analyze the preliminary operative report and the surgical footage to identify the at least one inconsistency between the preliminary operative report and the surgical footage. In some examples, step 1006 may use a multi-modal foundation model to analyze the preliminary operative report and the surgical footage to identify the at least one inconsistency between the preliminary operative report and the surgical footage (for example with a suitable textual prompt, such as ‘which surgical events listed in the attached preliminary operative report are not depicted in the attached surgical footage’, and/or ‘which significant surgical events that are typically described in an operative report are depicted in the attached surgical footage and are not indicated in the attached preliminary operative report’).

In some examples, step 1006 may identify a first mathematical object in a mathematical space, the first mathematical object corresponds to at least part of the preliminary operative report, for example as described above. Further, step 1006 may calculate a convolution of at least part of the surgical footage to obtain a numerical value. Further, step 1006 may calculate a function of the first mathematical object and the numerical value to obtain a second mathematical object in the mathematical space, for example as described above. Further, step 1006 may base the identification of the at least one inconsistency between the preliminary operative report and the surgical footage on the second mathematical object. For example, when the second mathematical object is in a selected group of mathematical objects (for example, in a selected region of the mathematical space), a specific inconsistency between the preliminary operative report and the surgical footage may be identified, and when the second mathematical object is not in the selected group of mathematical objects (for example, outside the selected region of the mathematical space), the specific inconsistency may not be identified.

In some examples, process 1000 may access a textual medical history of a patient (for example, of the patient of step 1002, of a different patient, and so forth). For example, the textual medical history may be textual medical history created before the surgical procedure (for example, at least one day before, at least one year before, and so forth). For example, the textual medical history may be read from a digital memory unit, may be received from an external computing device (for example, using a digital communication device), may be generated automatically, may be accessed in an Electronic Medical Record (EMR) or an Electronic Health Record (EHR) associated with the patient, may be received from a health care provided (for example, via a user interface), and so forth. Further, step 1006 may analyze the preliminary operative report, the surgical footage and the textual medical history of the patient to identify the at least one inconsistency between the preliminary operative report and the surgical footage. For example, step 1006 may use the textual medical history of the patient as the additional information in the machine learning model described above in relation to step 1006. In another example, step 1006 may use a multi-modal foundation model to analyze the preliminary operative report, the surgical footage and the textual medical history to identify the at least one inconsistency between the preliminary operative report and the surgical footage (for example with a suitable textual prompt, such as ‘which surgical events listed in the attached preliminary operative report are not depicted in the attached surgical footage and are inconsistence with the attached textual medical history’). One non-limiting example of such inconsistency may include a specific surgical action on a specific anatomical structure (such as the appendix) identified in the preliminary operative report, while it is impossible to determine a condition of the specific anatomical structure from the surgical footage (for example, when the surgical footage does not include the region of the specific anatomical structure, or where the view of the specific anatomical structure in the surgical footage is insufficient for such determination), and the textual medical history specifies a prior condition of the specific anatomical structure that is inconsistence with the specific surgical action (for example, where the textual medical history indicates that the appendix was removed in a previous surgical procedure).

In some examples, process 1000 may access an historic medical image of a patient (for example, of the patient of step 1002, of a different patient, and so forth). For example, the historic medical image may be a medical image of the patient captured before the surgical procedure (for example, at least one day before, at least one year before, and so forth). For example, the historic medical image may be read from a digital memory unit, may be received from an external computing device (for example, using a digital communication device), may be accessed in an EMR or an EHR associated with the patient, may be accessed in a Picture Archiving and Communication System (PACS), may be captured using at least one medical imaging device, and so forth. Further, step 1006 may analyze the preliminary operative report, the surgical footage and the historic medical image to identify the at least one inconsistency between the preliminary operative report and the surgical footage. For example, step 1006 may use the historic medical image of the patient as the additional information in the machine learning model described above in relation to step 1006. In another example, step 1006 may use a multi-modal foundation model to analyze the preliminary operative report, the surgical footage and the historic medical image to identify the at least one inconsistency between the preliminary operative report and the surgical footage (for example with a suitable textual prompt, such as ‘which surgical events listed in the attached preliminary operative report are not depicted in the attached surgical footage and are inconsistence with the attached historic medical image’). One non-limiting example of such inconsistency may include a characteristic of a specific anatomical structure (such as a size of a tumor) identified in the preliminary operative report, where it is impossible to determine the characteristic of the specific anatomical structure from the surgical footage (for example, when the surgical footage does not include the region of the specific anatomical structure, or where the view of the specific anatomical structure in the surgical footage is insufficient for such determination), and the historic medical image indicates a characteristic of the specific anatomical structure that is inconsistence with the characteristic identified in the preliminary operative report (for example, where the historic medical image shows that the tumor has a different size).

In some examples, process 1000 may access audio data recorded during a surgical procedure (for example, during the surgical procedure of step 1002, during a different surgical procedure, and so forth). For example, the audio data may be read from a digital memory unit, may be received from an external computing device (for example, using a digital communication device), may be accessed in a Picture Archiving and Communication System (PACS), may be captured using at least one audio sensor, and so forth. Further, step 1006 may analyze the preliminary operative report, the surgical footage and the audio data to identify the at least one inconsistency between the preliminary operative report and the surgical footage. For example, step 1006 may use the audio data as the additional information in the machine learning model described above in relation to step 1006. In another example, step 1006 may use a multi-modal foundation model to analyze the preliminary operative report, the surgical footage and the audio data to identify the at least one inconsistency between the preliminary operative report and the surgical footage (for example with a suitable textual prompt, such as ‘which surgical events listed in the attached preliminary operative report are not depicted in the attached surgical footage and are inconsistence with the attached audio data’). In one example, the audio data may include speech in a natural language produced by the health care provider, and the identification of the at least one inconsistency may be based on the speech. In another example, the audio data may include a conversation between the health care provider and another person, and the identification of the at least one inconsistency may be based on the conversation.

In some examples, the at least one inconsistency identified by step 1006 may include an event depicted in the surgical footage that is not reflected in the preliminary operative report. In some examples, the at least one inconsistency identified by step 1006 may include at least one of a surgical tool depicted in the surgical footage that is not reflected in the preliminary operative report, an inconsistency in a quantity of surgical staples, or an inconsistency in a color associated with surgical staples. In some examples, the at least one inconsistency identified by step 1006 may include a surgical tool depicted in the surgical footage that is not reflected in the preliminary operative report. In some examples, the at least one inconsistency identified by step 1006 may include at least one of inconsistency in a quantity of surgical staples, or inconsistency in a color associated with surgical staples. In some examples, the at least one inconsistency identified by step 1006 may include a secondary surgical trauma depicted in the surgical footage but not reflected in the preliminary operative report. A secondary surgical trauma may be an unintentional or unexpected damage that may occur during surgery. For example, secondary surgical trauma may be a result of complications and/or of surgical errors. In some examples, the identified at least one inconsistency by step 1006 may include at least one of an anatomical structure depicted in the surgical footage in a condition that is not reflected in the preliminary operative report, or an anatomical structure depicted in the surgical footage in a size that is different from a size of the anatomical structure reflected in the preliminary operative report. In some examples, the at least one inconsistency identified by step 1006 may include an anatomical structure depicted in the surgical footage in a condition that is not reflected in the preliminary operative report. In some examples, the at least one inconsistency identified by step 1006 may include an anatomical structure depicted in the surgical footage in a size that is different from a size of the anatomical structure reflected in the preliminary operative report. In some examples, the at least one inconsistency identified by step 1006 may include an event indicated in the preliminary operative report that is not depicted in the surgical footage. In some examples, the at least one inconsistency identified by step 1006 may include a surgical tool indicated in the preliminary operative report that is not depicted in the surgical footage.

In some examples, the at least one inconsistency identified by step 1006 may include an event depicted in the surgical footage that is not reflected in the preliminary operative report. For example, the surgical footage may depict a ligation of the right gonadal vein, while the preliminary operative report may not indicate the ligation, or may wrongly indicate that the right gonadal vein was spared. In some examples, step 1006 may analyze the preliminary operative report and/or the surgical footage to determine that a first event is depicted in the surgical footage and is not reflected in the preliminary operative report, and/or to determine that a second event is depicted in the surgical footage and is not reflected in the preliminary operative report (for example, using a LLM or an NLP algorithm to determine that the surgical events are not indicated in the preliminary operative report, and a visual event detection algorithm to determine that the surgical events are depicted in the surgical footage). Further, step 1006 may analyze the surgical footage to determine a characteristic of the first event, and/or to determine a characteristic of the second event (for example, using a visual classification algorithm). For example, step 1006 may, based on the characteristic of the first event, include the first event in the identified at least one inconsistency, and/or may, based on the characteristic of the second event, exclude the second event from the identified at least one inconsistency. For example, the characteristic of the first event may be a type of the first event, and/or the characteristic of the second event may be a type of the second event. In another example, the characteristic of the first event may be a magnitude of the first event, and/or the characteristic of the second event may be a magnitude of the second event. For example, the first event may be included in the identified at least one inconsistency in response to the magnitude of the first event being larger than a selected threshold, and the second event may be excluded from the identified at least one inconsistency in response to the magnitude of the second event being smaller than the selected threshold.

In some examples, the at least one inconsistency identified by step 1006 may include a surgical tool depicted in the surgical footage that is not reflected in the preliminary operative report. For example, the surgical footage may depict usage of an intraoperative ultrasound, and the preliminary operative report may not indicate that an intraoperative ultrasound was used. In some examples, step 1006 may analyze the preliminary operative report and/or the surgical footage to determine that a first surgical tool is depicted in the surgical footage and is not reflected in the preliminary operative report, and/or to determine that a second surgical tool is depicted in the surgical footage and is not reflected in the preliminary operative report (for example, using a LLM or an NLP algorithm to determine that the surgical tools are not indicated in the preliminary operative report, and a visual object detection algorithm to determine that the surgical tools are depicted in the surgical footage). Further, step 1006 may analyze the surgical footage to determine a characteristic of the first surgical tool, and/or to determine a characteristic of the second surgical tool (for example, using a visual classification algorithm). Further, step 1006 may, based on the characteristic of the first surgical tool, include the first surgical tool in the identified at least one inconsistency, and/or may, based on the characteristic of the second surgical tool, exclude the second surgical tool from the identified at least one inconsistency. In one example, the characteristic of the first surgical tool may be a type of the first surgical tool, and/or the characteristic of the second surgical tool may be a type of the second surgical tool. In another example, the characteristic of the first surgical tool may be a quantity associated with the first surgical tool, and/or the characteristic of the second surgical tool may be a quantity associated with the second surgical tool. In yet another example, the characteristic of the first surgical tool may be a size of the first surgical tool, and/or the characteristic of the second surgical tool may be a size of the second surgical tool. In an additional example, the characteristic of the first surgical tool may be a color associated with the first surgical tool, and/or the characteristic of the second surgical tool may be a color associated with the second surgical tool. For example, the first surgical tool may be included in the identified at least one inconsistency in response to the quantity associated with the first surgical tool being larger than a selected threshold, and the second surgical tool may be excluded from the identified at least one inconsistency in response to the quantity associated with the second surgical tool being smaller than the selected threshold.

In some examples, the at least one inconsistency identified by step 1006 may include an event indicated in the preliminary operative report that is not depicted in the surgical footage. For example, the preliminary operative report may indicate that a critical view of safety was reached, while the surgical footage may show that the cystic duct was not visible. In some examples, step 1006 may analyze the preliminary operative report and/or the surgical footage to determine that a first event is indicated in the preliminary operative report and is not depicted in the surgical footage, and/or to determine that a second event is indicated in the preliminary operative report and is not depicted in the surgical footage (for example, using a LLM or an NLP algorithm to determine that the surgical events are indicated in the preliminary operative report, and a visual event detection algorithm to determine that the surgical events are not depicted in the surgical footage). Further, step 1006 may analyze the preliminary operative report to determine a characteristic of the first event, and/or to determine a characteristic of the second event, for example as described above (for example, using a LLM or an NLP algorithm). Further, step 1006 may, based on the characteristic of the first event, include the first event in the identified at least one inconsistency, and/or may, based on the characteristic of the second event, exclude the second event from the identified at least one inconsistency. In one example, the characteristic of the first event may be a type of the first event, and/or the characteristic of the second event may be a type of the second event. In another example, the characteristic of the first event may be a magnitude of the first event, and/or the characteristic of the second event may be a magnitude of the second event. For example, the first event may be included in the identified at least one inconsistency in response to the magnitude of the first event being larger than a selected threshold, and the second event may be excluded from the identified at least one inconsistency in response to the magnitude of the second event being smaller than the selected threshold.

In some examples, the at least one inconsistency identified by step 1006 may include a surgical tool indicated in the preliminary operative report that is not depicted in the surgical footage. For example, the preliminary operative report may indicate the usage of a biological mesh, while the surgical footage may depict no usage of a mesh, or a usage of a synthetic mesh. In some examples, step 1006 may analyze the preliminary operative report and/or the surgical footage to determine that a first surgical tool is indicated in the preliminary operative report and is not depicted in the surgical footage, and/or to determine that a second surgical tool is indicated in the preliminary operative report and is not depicted in the surgical footage (for example, using a LLM or an NLP algorithm to determine that the surgical tools are indicated in the preliminary operative report, and a visual object detection algorithm to determine that the surgical tools are not depicted in the surgical footage). Further, step 1006 may analyze the preliminary operative report to determine a characteristic of the first surgical tool, and/or to determine a characteristic of the second surgical tool (for example, using a LLM or an NLP algorithm). Further, step 1006 may, based on the characteristic of the first surgical tool, include the first surgical tool in the identified at least one inconsistency, and/or may, based on the characteristic of the second surgical tool, exclude the second surgical tool from the identified at least one inconsistency. In one example, the characteristic of the first surgical tool may be a type of the first surgical tool, and/or the characteristic of the second surgical tool may be a type of the second surgical tool. In another example, the characteristic of the first surgical tool may be a quantity associated with the first surgical tool, and/or the characteristic of the second surgical tool may be a quantity associated with the second surgical tool. In some examples, the characteristic of the first surgical tool may be a size of the first surgical tool, and/or the characteristic of the second surgical tool may be a size of the second surgical tool. In some examples, the characteristic of the first surgical tool may be a color associated with the first surgical tool, and/or the characteristic of the second surgical tool may be a color associated with the second surgical tool. For example, the first surgical tool may be included in the identified at least one inconsistency in response to the quantity associated with the first surgical tool being larger than a selected threshold, and the second surgical tool may be excluded from the identified at least one inconsistency in response to the quantity associated with the second surgical tool being smaller than the selected threshold.

In some examples, the at least one inconsistency identified by step 1006 may be based on a noun and an adjective adjacent to the noun in the preliminary operative report. For example, the at least one inconsistency identified by step 1006 may be based on a characteristic of an element in the surgical footage indicated by the noun being inconsistence with the adjective. For example, step 1006 may analyze the preliminary operative report and/or the surgical footage to identify that a characteristic of an element in the surgical footage indicated by the noun is inconsistence with the adjective, for example using a multi-modal foundation model (for example with a suitable textual prompt, such as ‘is the element corresponding to this noun, {the-noun}, has a characteristic corresponding to this adjective, {the-adjective}, in the attached surgical footage?’) In one example, the preliminary operative report may include ‘small tumor’ while the surgical footage may depict a large tumor. In some examples, the at least one inconsistency identified by step 1006 may be based on a verb and an adverb adjacent to the verb in the preliminary operative report. For example, the at least one inconsistency identified by step 1006 may be based on a characteristic of a surgical action indicated by the verb being inconsistence with the adverb. For example, the characteristic of the surgical action may be an extent of the surgical action. In another example, the characteristic of the surgical action may be a timing of the surgical action. In yet another example, the characteristic of the surgical action may be a type of the surgical action. In one example, step 1006 may analyze the preliminary operative report and/or the surgical footage to identify that a characteristic of a surgical action in the surgical footage indicated by the verb is inconsistence with the adjective, for example using a multi-modal foundation model (for example with a suitable textual prompt, such as ‘is the surgical action corresponding to this verb, {the-verb}, has a characteristic corresponding to this adverb, {the-adverb}, in the attached surgical footage?’)

In some examples, the at least one inconsistency identified by step 1006 may be based on an order of events in the surgical footage being different from an order of the events indicated in the preliminary operative report. In some examples, the at least one inconsistency identified by step 1006 may be based on a timing of an event in the surgical footage being different from a timing of the event indicated in the preliminary operative report. Some non-limiting examples of such timing of an event may include timing relative to another event, timing relative to a common timeline of the surgical procedure (such as time measured from the beginning of the surgical procedure, time measured from the beginning of the surgical footage, etc.), and so forth. In some examples, the at least one inconsistency identified by step 1006 may include an inconsistency in a duration of a phase of the surgical procedure (for example, an inconsistency in a Colonoscopy Withdrawal Time (CWT) of a colonoscopy). In some examples, the at least one inconsistency identified by step 1006 may include at least one of an inconsistency in a surgical technique or an inconsistency a type of procedure consistence with the surgical footage that is not indicated by the preliminary operative report. In some examples, the at least one inconsistency identified by step 1006 may include an inconsistency in a surgical technique. Some non-limiting examples of such surgical technique may include laparoscopic surgery, robotic surgery, open surgery, and so forth. In some examples, the preliminary operative report may indicate at least one type of procedure, and the at least one inconsistency identified by step 1006 may include another type of procedure consistence with the surgical footage but not indicated by the preliminary operative report. For example, the preliminary operative report may indicate ‘Robotic-assisted Radical Prostatectomy’ but not ‘Robotic-assisted Inguinal Hernia Repair’, while the surgical footage may be consistence with both.

In some examples, step 1008 may comprise providing an indication of at least one inconsistency (for example, of the at least one inconsistency identified by step 1006, of at least one inconsistency between a preliminary operative report and a surgical footage, of different inconsistencies, etc.), for example as described above or as described in relation to step 1308. For example, step 1008 may provide a textual indication of the at least one inconsistency, may provide a visual indication of the at least one inconsistency, may provide an audible indication of the at least one inconsistency, may provide an indication in a natural language of the at least one inconsistency, may provide a graphical indication of the at least one inconsistency, and so forth. In one example, step 1008 may provide the indication to a health care provider (for example, to the health care provider of step 1002, to a different health care provider, and so forth).

In some examples, the indication of the identified at least one inconsistency provided by step 1008 may include an output in a natural language explaining the identified at least one inconsistency. For example, step 1008 may use a LLM to analyze data associated with the identified at least one inconsistency and generate the output in the natural language explaining the identified at least one inconsistency. In another example, step 1008 may insert information associated with the identified at least one inconsistency into a template (for example, inserting sizes into the template ‘The operative report indicate that the size of the tumor is {size-based-on-operative-report} while the surgical footage shows that the actual size is {size-based-on-surgical-footage}’) to generate the output in the natural language explaining the identified at least one inconsistency. In one example, step 1008 may provide the output in the natural language textually and/or audibly. In one example, the indication of the identified at least one inconsistency provided by step 1008 may include a question in a natural language indicative of the identified at least one inconsistency. For example, the preliminary operative report may not indicate any injury to the bowel, the surgical footage may depict an injury to the bowel, and the question may include ‘Was the bowel injured? How would you characterize the injury?’ In one example, the question may be provided while displaying a portion of the surgical footage depicting the injury.

In some examples, the indication of the identified at least one inconsistency provided by step 1008 may include a suggestion of a modification to the preliminary operative report to correct the at least one inconsistency. In one example, the modification may include at least one of (i) a replacement of a first adjective by a second adjective in the preliminary operative report, (ii) an automatically generated text and a position in the preliminary operative report for an insertion of the automatically generated text, or (iii) a removal of an automatically selected portion of the preliminary operative report. In one example, the modification may include a replacement of a first adjective by a second adjective in the preliminary operative report (for example, changing ‘minimal bleeding’ to ‘major bleeding’, changing ‘small tumor’ to ‘large tumor’, changing ‘effective’ to ‘ineffective’, or changing ‘clean incision’ to ‘contaminated incision’). In another example, the modification may include a replacement of a first adverb by a second adverb in the preliminary operative report (for example, changing ‘successfully’ to ‘unsuccessfully’, or changing ‘effectively’ to ‘ineffectively’). In yet another example, the suggested modification may include an automatically generated text and/or a position in the preliminary operative report for an insertion of the automatically generated text. In an additional example, the suggested modification may include a removal of an automatically selected portion of the preliminary operative report. In yet another example, the suggested modification may include a modification of an automatically selected portion of a text included in the preliminary operative. In one example, the suggested modification may be automatically generated, for example using step 1314. In another example, the suggested modification may be received from an external process (for example, via a digital communication device, via a memory unit, etc.) or from an individual (for example, via a user interface). In one example, the suggested modification may be presented, for example to an individual, for example using step 1316.

In some examples, the indication of the identified at least one inconsistency provided by step 1008 may include a suggestion of a modification to the preliminary operative report to correct the at least one inconsistency, and the suggested modification may include an automatically generated text and/or a position in the preliminary operative report for an insertion of the automatically generated text, for example as described herein. In one example, the automatically generated text may be generated using a LLM or a multi-modal foundation model, for example by analyzing the preliminary operative report and/or the surgical footage (for example, with a suitable textual prompt, such as ‘generate a text describing the event depicted in the attached segment of the surgical footage’). In another example, the automatically generated text may be generated based on an analysis of the preliminary operative report and the surgical footage. In one example, the automatically generated text may identify an event depicted in the surgical footage that is not reflected in the preliminary operative report. In one example, wherein the automatically generated text may indicate a characteristic of the event determined by analyzing the surgical footage. In one example, the automatically generated text may identify a surgical tool depicted in the surgical footage that is not reflected in the preliminary operative report. In one example, the automatically generated text may indicate a characteristic of the surgical tool determined by analyzing the surgical footage. In one example, the automatically generated text may indicate a condition of an anatomical structure depicted in the surgical footage. In one example, a LLM or a multi-modal foundation model may analyze the preliminary operative report to identify the position in the preliminary operative report for the insertion of the automatically generated text (for example, with a suitable textual prompt, such as ‘select a position in the attached preliminary operative report for an insertion of the following text {the automatically generated text}’). In another example, the position in the preliminary operative report for the insertion of the automatically generated text may be selected based on a sequence of events in the surgical footage. For example, the automatically generated text may be associated with a specific event depicted in the surgical footage, and the identified position may be after an event preceding the specific event in the surgical footage, and/or before an event succeeding the specific event in the surgical footage.

In some examples, the indication of the identified at least one inconsistency provided by step 1008 may include a suggestion of a modification to the preliminary operative report to correct the at least one inconsistency, and the suggested modification may include a removal of an automatically selected portion of the preliminary operative report. In one example, the automatically selected portion may be selected using a LLM or a multi-modal foundation model, for example by analyzing the preliminary operative report and/or the surgical footage (for example, with a suitable textual prompt, such as ‘identify a portion of the attached preliminary operative report that describes an event that is not depicted in the attached surgical footage’). In another example, the automatically selected portion may be selected based on an analysis of the preliminary operative report and the surgical footage. In an additional example, the automatically selected portion may indicate an event that is not depicted in the surgical footage. In yet another example, the automatically selected portion may indicate a surgical tool that is not depicted in the surgical footage.

In some examples, the indication of the identified at least one inconsistency provided by step 1008 may include a suggestion of a modification to the preliminary operative report to correct the at least one inconsistency, and the suggested modification may include a modification of an automatically selected portion of a text included in the preliminary operative report. In one example, the automatically selected portion may be selected using a LLM or a multi-modal foundation model, for example by analyzing the preliminary operative report and/or the surgical footage (for example, with a suitable textual prompt, such as ‘identify a portion of the attached preliminary operative report that describes a characteristic of a specific event that is inconsistence with a characteristic of the event in the attached surgical footage’). In another example, the automatically selected portion may be selected based on an analysis of the preliminary operative report and the surgical footage. In one example, the suggested modification may change at least one of a characteristic of an event depicted in the surgical footage, a characteristic of a surgical tool depicted in the surgical footage, or a characteristic of an anatomical structure depicted in the surgical footage. In one example, the modification to the preliminary operative report may be determined using a LLM or a multi-modal foundation model, for example by analyzing the preliminary operative report and/or the surgical footage (for example, with a suitable textual prompt, such as ‘modify this portion of the attached preliminary operative report to reflect the characteristic of the event in the attached segment of the surgical footage’). In another example, the modification to the preliminary operative report may include a modification of an adjective and/or a modification of an adverb (for example as described above).

In some examples, step 1006 may analyze the preliminary operative report and/or the surgical footage (for example, using a trained machine learning model, using an artificial neural network, using a multi-modal foundation model, etc.) to identify a portion of the preliminary operative report associated with the identified at least one inconsistency and/or a portion of the surgical footage associated with the identified at least one inconsistency. For example, the trained machine learning model may be a machine learning model trained using training examples to identify portions of operative reports and/or portions of surgical footages associated with inconsistencies between operative reports and surgical footages. An example of such training example may include a sample operative report, a sample surgical footage, and an indication of a sample inconsistency between the sample operative report and the sample surgical footage, together with a label indicating a portion of the sample operative report and/or a portion of the sample surgical footage associated with the sample inconsistency. In another example, the multi-modal foundation model may be used with a suitable textual prompt, such as ‘identify the portions of the attached preliminary operative report and portions of the attached surgical footage associated with {a textual description of the inconsistency}’). Further, step 1008 may provide an indication of the identified portion of the preliminary operative report and/or an indication of the identified portion of the surgical footage. For example, step 1008 may display a visual indication of the identified portion of the preliminary operative report while displaying the identified portion of the surgical footage, for example as described in relation to step 1208.

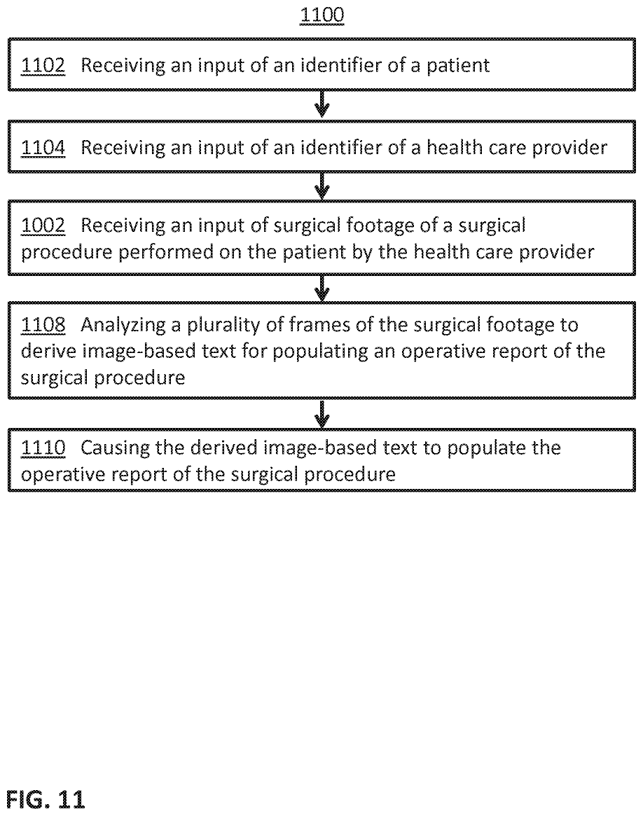

FIG. 11 is a flow diagram of an exemplary process 1100, consistent with some embodiments of the present disclosure. In this example, process 1100 may comprise receiving an input of an identifier of a patient (step 1102); receiving an input of an identifier of a health care provider (step 1104); receiving an input of surgical footage of a surgical procedure performed on the patient by the health care provider (step 1002); analyzing a plurality of frames of the surgical footage to derive image-based text for populating an operative report of the surgical procedure (step 1108); and causing the derived image-based text to populate the operative report of the surgical procedure (step 1110). In other examples, process 1100 may include additional steps or fewer steps. In other examples, one or more steps of process 1100 may be executed in a different order and/or one or more groups of steps may be executed simultaneously.

In some examples, a system may include at least one processing unit configured to perform process 1100 and/or one or more of the steps of process 1100. In one example, the system may further comprise a digital communication device configured to receive an input of surgical footage of a surgical procedure (such as the surgical footage of step 1002, a surgical footage of a surgical procedure performed on a patient by a health care provider, and so forth), and/or to receive an identifier of a patient (such as the identifier of the patient of step 1102, an identifier of a different patient, and so forth), and/or to receive an identifier of a health care provider (such as the identifier of the health care provider of step 1104, an identifier of a different health care provider, and so forth). In one example, the system may further comprise at least one image sensor configured to capture surgical footage of a surgical procedure (such as the surgical footage of step 1002, a surgical footage of a surgical procedure performed on a patient by a health care provider, and so forth). In one example, the system may further comprise at least one digital memory unit configured to store surgical footage of a surgical procedure (such as the surgical footage of step 1002, a surgical footage of a surgical procedure performed on a patient by a health care provider, and so forth), and/or an identifier of a patient (such as the identifier of the patient of step 1102), and/or an identifier of a health care provider (such as the identifier of the health care provider of step 1104). Further, the at least one processing unit may be configured to read the surgical footage and/or the identifiers from the at least one digital memory unit. In some examples, a non-transitory computer readable medium may store computer implementable instructions that when executed by at least one processor may cause the at least one processor to perform operations, and the operations may include process 1100 and/or one or more of the steps of process 1100. In some examples, a method may include performing the steps of process 1100.

In some examples, step 1102 may comprise receiving an input of an identifier of a patient. For example, step 1102 may use step 911 to receive the identifier of the patient. In another example, step 1102 may read the identifier of the patient from a digital memory unit, may receive the identifier of the patient from an external computing device (for example, using a digital communication device, from a Hospital Information System (HIS), from an EMR or an EHR, etc.), may determine the identifier of the patient (for example, by analyzing an image of a physical medical document, for example using an Optical Character Recognition (OCR) algorithm, etc.), and so forth.

In some examples, step 1104 may comprise receiving an input of an identifier of a health care provider. For example, step 1104 may use step 913 to receive the identifier of the health care provider. In another example, step 1102 may read the identifier of the health care provider from a digital memory unit, may receive the identifier of the health care provider from an external computing device (for example, using a digital communication device, from an HIS, from an EMR or an EHR, etc.), may determine the identifier of the health care provider (for example, by analyzing an image of a physical medical document, for example using an Optical Character Recognition (OCR) algorithm, etc.), and so forth.

In some examples, step 1108 may comprise analyzing a plurality of frames of a surgical footage (such as, the surgical footage received by step 1002, the surgical footage received by step 915, the surgical video accessed by step 510, a different surgical footage, etc.) to derive image-based text for populating an operative report of a surgical procedure (for example, a surgical procedure associated with the surgical footage, the surgical procedure of step 1002, the surgical procedure of step 915, the surgical procedure of step 510, a different surgical procedure, etc.), for example as described above. In one example, step 1108 may use step 917 to analyze the plurality of the frames of the surgical footage to derive the image-based text for populating the operative report. In some examples, step 1108 may use a trained machine learning model to analyze the plurality of frames of the surgical footage to derive the image-based text for populating the operative report. The trained machine learning model may be a machine learning model trained using training examples to generate text for operative reports from visual information and/or additional information. An example of such training example may include sample frames of a sample surgical procedure and/or additional information associated with the sample surgical procedure, together with a sample text for a sample operative report of the sample surgical procedure. In some examples, step 1108 may use an artificial neural network to analyze the plurality of frames of the surgical footage to derive the image-based text for populating the operative report. For example, the artificial neural network may be configured to analyze videos to derive textual descriptions. In some examples, step 1108 may use a multi-modal foundation model to analyze the plurality of frames of the surgical footage and/or additional information associated with the surgical procedure to derive image-based text for populating the operative report (for example with a suitable textual prompt, such as ‘Attached is a video from a surgical procedure and a document with additional information associated with the surgical procedure. Generate a textual description describing the surgical events’).

In some examples, step 1108 may calculate a convolution of a part of the surgical footage to obtain a numerical result value (for example, calculate a convolution a first part of the surgical footage to obtain a first numerical result value, calculate a convolution of a second part of the surgical footage to obtain a second numerical result value, and calculate a convolution of a third part of the surgical footage to obtain a third numerical result value). Further, step 1108 may calculate a function of the first numerical result value and the second numerical result value to obtain a first mathematical object, for example as described above. Further, step 1108 may calculate a function of the first numerical result value and the third numerical result value to obtain a second mathematical object, for example as described above. Further, step 1108 may select an adjective based on the first mathematical object, for example as described above. Further, step 1108 may select a noun based on the second mathematical object, for example as described above. Further, step 1108 may include the adjective and the noun in the derived image-based text. In one example, the adjective may be adjacent to the noun in the derived image-based text. In another example, at least one sentence may separate the adjective from the noun in the derived image-based text.

In some examples, the image-based text derived by step 1108 may include a noun indicative of an anatomical structure depicted in the surgical footage, and an adjective adjacent to the noun. The adjective may be indicative of a characteristic of the anatomical structure. In one example, the characteristic of the anatomical structure may be a size of the anatomical structure. In another example, the characteristic of the anatomical structure may be a condition of the anatomical structure. For example, the derived image-based text may include ‘fatty liver’, ‘enlarged prostate’, or ‘inflamed appendix’. In some examples, the image-based text derived by step 1108 may include a noun indicative of an event depicted in the surgical footage, and an adjective adjacent to the noun. The adjective may be indicative of a characteristic of the event. For example, the derived image-based text may include ‘clean incision’, ‘inadvertent injury’, ‘wide excision’ and so forth. In some examples, the image-based text derived by step 1108 may include a noun indicative of a surgical tool depicted in the surgical footage, and an adjective adjacent to the noun. The adjective may be indicative of a characteristic of the surgical tool. In one example, the characteristic of the surgical tool may be a type of the surgical tool. In another example, the characteristic of the surgical tool may be a color associated with the surgical tool. In yet another example, the characteristic of the surgical tool may be a quantity associated with the surgical tool. For example, the derived image-based text may include ‘large hemostat’ or ‘blue staples’. In some examples, the image-based text derived by step 1108 may include a verb indicative of a surgical action depicted in the surgical footage, and an adverb adjacent to the verb. The adverb may be indicative of a characteristic of the surgical action. In one example, the characteristic of the surgical action may be an extent of the surgical action. In another example, the characteristic of the surgical action may be a timing of the surgical action. In yet another example, the characteristic of the surgical action may be a type of the surgical action. For example, the derived image-based text may include ‘superficially incising’ or ‘strongly retracting’. In some examples, the image-based text derived by step 1108 may be indicative of at least one of a surgical technique depicted in the surgical footage or of a plurality of types of the surgical procedure depicted in the surgical footage. In some examples, the image-based text derived by step 1108 may be indicative of a surgical technique depicted in the surgical footage. For example, step 1108 may analyze the surgical footage using a visual classification algorithm to determine the surgical technique, and may include a textual indication of the determined surgical technique in the derived image-based text. Some non-limiting examples of such surgical technique may include ‘Open surgery’, ‘Minimal invasive surgery’, ‘Laparoscopic surgery’, ‘Robotic surgery’, ‘Endoscopic surgery’, ‘Arthroscopic surgery’, ‘Bronchoscopic surgery’, ‘Thoracoscopic surgery’, ‘Hysteroscopic surgery’, and so forth. In some examples, the image-based text derived by step 1108 may be indicative of at least one type of surgical procedure (for example, a plurality of types of the surgical procedure, at least three types of the surgical procedure, at least five types of the surgical procedure, etc.) depicted in the surgical footage. For example, step 1108 may analyze the surgical footage using a visual multi-class classification algorithm to determine the at least one type of surgical procedure, and may include a respective textual indication of each determined type of surgical procedure in the derived image-based text. For example, the derived image-based text may include ‘Robotic-assisted Radical Prostatectomy’ and ‘Robotic-assisted Inguinal Hernia Repair’. In some examples, the image-based text derived by step 1108 may be indicative of a duration of a phase of the surgical procedure (for example, of a CWT of a colonoscopy). For example, step 1108 may analyze the surgical footage to identify the timing of the starting and ending of the phase to determine the duration of the phase, and may include a textual indication of the duration (such as ‘2 minutes’, ‘longer than average’, and so forth). In some examples, the image-based text derived by step 1108 may include an indication that a specific surgical action was not performed during the surgical procedure. In one example, the indication that the specific surgical action was not performed during the surgical procedure may include at least one word, may include at least one phrase, may include a negation, and so forth. For example, the derived image-based text may include ‘a critical view of safety was not reached’, or ‘the right gonadal vein was spared’ (indicating that no ligation of the right gonadal vein was performed). In some examples, the image-based text derived by step 1108 may include an indication of at least one intraoperative complication determined based on an analysis of the plurality of frames. Some non-limited examples of such intraoperative complications may include hemorrhagic complications, cardiovascular complications, respiratory complications, equipment-related complications, severe bleeding, hematoma formation, organ injury, burn injury, organ perforation, foreign body retention, and so forth. In some examples, the image-based text derived by step 1108 may include at least a first portion and a second portion. The first portion may include summarized key details observed during the surgical procedure, and/or the second portion may include detailed description of the surgical procedure.

In some examples, step 1108 may analyze the plurality of frames of the surgical footage to identify a first intraoperative event and a second intraoperative event, for example using a visual event detection algorithm. Further, step 1108 may determine a first level of implication associated with the first intraoperative event, and/or may determine a second level of implication associated with the second intraoperative event. For example, step 1108 may analyze the plurality of frames of the surgical footage and/or additional information associated with the surgical procedure to determine the first and second levels of implication (for example, using a classification algorithm where each class corresponds to a different level, using a data regression model, and so forth). Further, step 1108 may, based on the first level of implication, exclude any indication of the first intraoperative event from the derived image-based text, and/or may, based on the second level of implication, include an indication of the second intraoperative event in the derived image-based text. In one example, the first and second levels of implication may be levels of implications for clinical care. For example, the first event may be a minor bleeding event, the second event may be a massive hemorrhage, the first level of implication for clinical care may be low, the second level of implication for clinical care may be high, and in response step 1108 may exclude the first event from the derived image-based text and may include the second event in the derived image-based text. In another example, the first and second levels of implication may be levels of implications for billing. For example, the first event may be a minor bleeding event controlled with electrocautery, the second event may be a cardiac arrest requiring intraoperative resuscitation, the first level of implication for billing may be low, the second level of implication for billing may be high, and in response step 1108 may exclude the first event from the derived image-based text and may include the second event in the derived image-based text. In one example, step 1108 may, based on the first and second levels of implication, exclude any indication of the first intraoperative event from the derived image-based text, and/or may, based on the first and second levels of implication, include an indication of the second intraoperative event in the derived image-based text. For example, when the two events are two bleeding events, when the second event is significantly more severe bleeding (and therefore the second level of implication is significantly higher than the first level of implication), step 1108 may exclude the first event from the derived image-based text and may include the second event in the derived image-based text, and when the two bleeding events are of similar severity (and therefore the first and second levels of implication are similar), step 1108 may include both the first and second events in the derived image-based text.

In some examples, step 1108 may analyze the plurality of frames to identify a plurality of events (such as a first surgical event, a second surgical event, and a third surgical event), for example using a visual event detection algorithm. Further, step 1108 may analyze the plurality of frames to determine a temporal order of the plurality of events (for example, a temporal order of the first, second and third surgical events), for example based on an order of the frames in which the events were detected. Further, step 1108 may include in the derived image-based text an indication of the temporal order of the plurality of events (such as an indication of the temporal order of the first, second and third surgical events). In one example, the image-based text derived by step 1108 may list the plurality of events in an order corresponding to the temporal order. In another example, the image-based text derived by step 1108 may include adverbs selected based on the temporal order (such as ‘after’, ‘before’, ‘later’, and so forth) to indicate the temporal order.

In some examples, step 1108 may derive the image-based text in a style selected based on the health care provider. For example, the derived image-based text may include phrases typically used by the health care provider. In another example, the derived image-based text may include or omit details based on a pattern of details inclusion or exclusion typical to the health care provider. In yet another example, a length the derived image-based text may be based on a typical length of operative reports created by the health care provider. In some examples, step 1108 may further comprise analyzing at least one historical operative report associated with the health care provider to determine the style. For example, step 1108 may use a LLM to analyze the at least one historical operative report to determine the style and/or to derive the image-based text in the determined style (for example, with a suitable textual prompt, such as ‘Attached are some details for an operative report. Also attached are some historical operative reports. Generate text for a new operative report based on the details, in a style corresponding to the historical operative reports’). In another example, step 1108 may use statistical analysis to analyze the at least one historical operative report to determine the style (for example, finding typical phrases, lengths, level of details, and so forth).

In some examples, step 1108 may approach the health care provider via a user interface to obtain a specific detail. The specific detail may not be available from the surgical footage. For example, step 1108 may analyze the plurality of frames of the surgical footage to determine one or more details not available from the surgical footage (for example, to determine that the specific detail is not available from the surgical footage), and may not approach the health care provider to obtain the one or more details. For example, the analysis may fail to determine an obesity classification of the patient from the surgical footage, and may approach the health care provider to obtain the obesity classification. Further, step 1108 may include information based on the specific detail in the derived image-based text. For example, step 1108 may include ‘uncommon in patients with healthy weight’ based on the obesity classification of the patient being ‘healthy weight’.

In some examples, step 1108 may access a textual medical history of a patient (for example, of the patient of step 1002, of the patient of step 1102, of a different patient, and so forth), for example as described above in relation to process 1000. For example, the textual medical history may be textual medical history created before the surgical procedure (for example, at least one day before, at least one year before, and so forth). Further, step 1108 may analyze the plurality of frames and the textual medical history of the patient to identify information from the textual medical history for integration into the operative report. For example, step 1108 may use the textual medical history of the patient as the additional information in the machine learning model described above in relation to step 1108. In another example, step 1108 may use the textual medical history of the patient as the additional information in the usage of the multi-modal foundation model described above in relation to step 1108.

In some examples, step 1108 may access an historic medical image of a patient (for example, of the patient of step 1002, of the patient of step 1102, of a different patient, and so forth), for example as described above in relation to process 1000. For example, the historic medical image may be a medical image of the patient captured before the surgical procedure (for example, at least one day before, at least one year before, and so forth). Further, step 1108 may analyze the plurality of frames and the historic medical image to determine information based on the historic medical image for integration into the operative report. For example, step 1108 may use the historic medical image as the additional information in the machine learning model described above in relation to step 1108. In another example, step 1108 may use the historic medical image as the additional information in the usage of the multi-modal foundation model described above in relation to step 1108.

In some examples, step 1108 may access audio data recorded during a surgical procedure (for example, during the surgical procedure of step 1002, during a different surgical procedure, and so forth). For example, the audio data may be read from a digital memory unit, may be received from an external computing device (for example, using a digital communication device), may be accessed in a Picture Archiving and Communication System (PACS), may be captured using at least one audio sensor, and so forth. Further, step 1108 may analyze the plurality of frames and/or the audio data to determine information based on the audio data for integration into the operative report. For example, step 1108 may use the audio data as the additional information in the machine learning model described above in relation to step 1108. In another example, step 1108 may use the audio data as the additional information in the usage of the multi-modal foundation model described above in relation to step 1108. In one example, the audio data may include speech in a natural language produced by the health care provider, and the determined information based on the audio data for integration into the operative report may be based on the speech. In another example, the audio data may include a conversation between the health care provider and another person, and the determined information based on the audio data for integration into the operative report may be based on the conversation.

In some examples, step 1108 may receive an indication of a pre-operative diagnosis associated with the patient. For example, step 1108 may read the pre-operative diagnosis from a digital memory unit, may receive the pre-operative diagnosis from an external computing device (for example, using a digital communication device), may access in an EMR or an EHR associated with the patient to obtain the pre-operative diagnosis, may receive the pre-operative diagnosis from the health care provider (for example, via a user interface), and so forth. Further, step 1108 may determine a post-operative diagnosis based on an analysis of the plurality of frames and the pre-operative diagnosis, wherein the post-operative diagnosis differs from the pre-operative diagnosis, and may include the post-operative diagnosis in the derived image-based text. In some examples, step 1108 may analyze the plurality of frames to determine the post-operative diagnosis. Further, step 1108 may include post-operative diagnosis in the derived image-based text. For example, step 1108 may use a machine learning model to analyze the plurality of frames and/or the pre-operative diagnosis to determine the post-operative diagnosis. The machine learning model may be a machine learning model trained using training examples to determine post-operative diagnosis based on pre-operative diagnosis and/or surgical videos. An example of such training example may include a sample pre-operative diagnosis associated with a sample surgical procedure and/or a sample surgical footage from the sample surgical procedure, together with a label indicative of a sample post-operative diagnosis associated with the sample surgical procedure. In another example, step 1108 may access a data-structure associating pre-operative diagnoses and surgical events with post-operative diagnoses, based on the pre-operative diagnosis and/or on events detected in the plurality of frames of the surgical footage, to determine the post-operative diagnosis. For example, the pre-operative diagnosis may include ‘Appendicitis’, based on severe abdominal pain, fever, and an inflamed appendix seen on imaging; the surgical footage shows a tumor in the colon that has perforated, mimicking appendicitis; and the post-operative diagnoses may include ‘Colorectal Cancer’. In another example, the pre-operative diagnosis may include ‘Benign Prostatic Hyperplasia’, based on urinary retention, weak stream, increased frequency, and mildly elevated Prostate-Specific Antigen (PSA) but within a range suggestive of BPH; the surgical footage may depict firm, irregular areas within the prostate that do not resemble typical BPH tissue; and the post-operative diagnoses may include ‘Primary Malignant Neoplasm of the Prostate’.

In some examples, step 1110 may comprise causing text (such as an image-based text, the image-based text derived by step 1108, the image-based text derived by step 917, different text, etc.) to populate an operative report of a surgical procedure (for example, the operative report of step 1108, an operative report of a surgical procedure associated with the surgical footage received by step 1002, an operative report of the surgical procedure of step 1002, an operative report of the surgical procedure of step 915, an operative report of the surgical procedure of step 510, a different operative report, and so forth). In one example, step 1110 may use step 919 to cause the text to populate the operative report. In one example, step 1110 may cause the text to populate a selected field of the operative report. In another example, step 1110 may analyze a preliminary operative report to select a position for an insertion of the text in the preliminary operative report (for example, selecting a position within a free-text included the preliminary operative report), for example as described above (for example in relation to step 1008). Further, step 1110 may cause the insertion of the text at the selected position. In one example, step 1110 may create a new operative report of the surgical procedure, and include the text in the new operative report. For example, the new operative report may include only the text. In another example, the new operative report may include the text and at least one other field. Additionally or alternatively, step 1110 may comprise associating the operative report with an EHR of the patient. For example, step 1110 may transmit digital information (for example, via a digital communication device) to an external computing device to cause the external computing device to associate the operative report with the EHR of the patient. Such digital information may include a digital encoding of the operative report and/or a digital identified of the operative report, may include an identifier of the patient, and so forth. In another example, step 1110 may manipulate at least one entry in a dataset and/or a database to associate the operative report with the EHR of the patient. In yet another example, step 1110 may manipulate the EHR of the patient to associate the operative report with the EHR of the patient.

In some examples, step 1110 may present the derived image-based text to the health care provider via a user interface that may enable the health care provider to modify the derived image-based text. For example, the user interface may enable the health care provider to modify the derived image-based text using a keyboard. In another example, the user interface may enable the health care provider to provide instructions in a natural language (for example, via text, via speech, etc.), and the derived image-based text may be automatically modified based on the instructions in the natural language. For example, the instructions may include ‘please add more details about the ligation’, a multi-modal foundation model may be used to analyze the instructions, the derived image-based text, and the plurality of frames of the surgical footage to change ‘The right gonadal vein was ligated before . . . ’ to ‘The right gonadal vein was ligated using a bipolar electrocautery device before . . . ’.

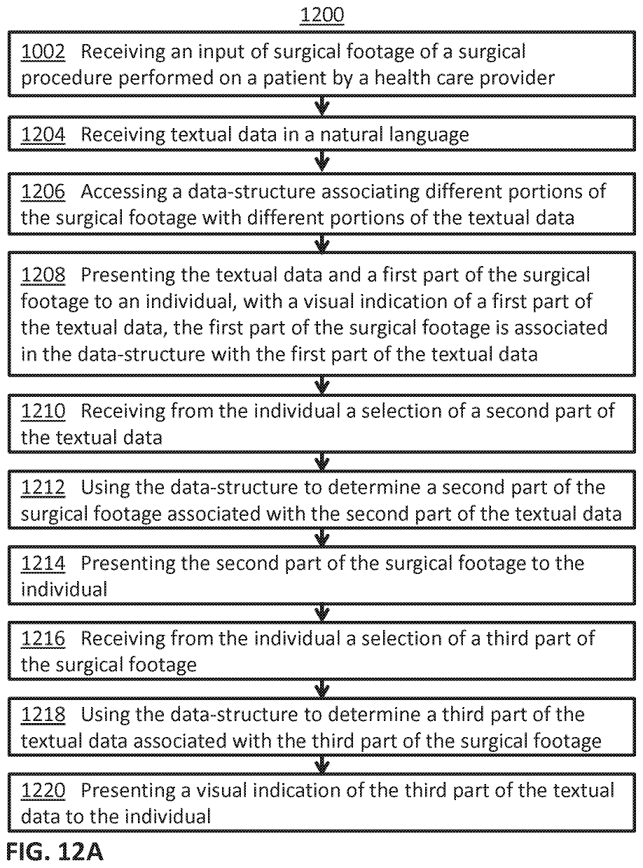

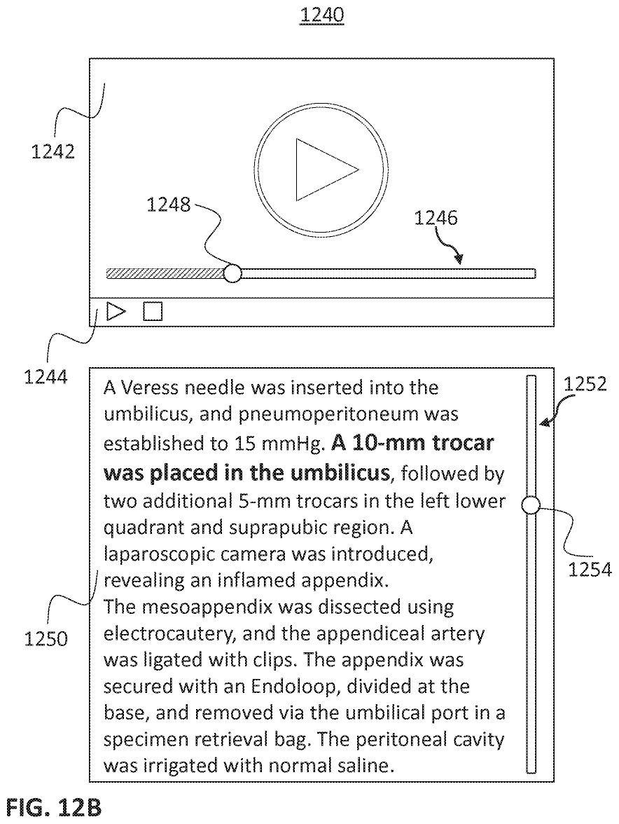

FIG. 12A is a flow diagram of an exemplary process 1200 for synchronized browsing of surgical videos and operative reports, consistent with some embodiments of the present disclosure. In this example, process 1200 may comprise receiving an input of surgical footage of a surgical procedure performed on a patient by a health care provider (step 1002); receiving textual data in a natural language (step 1204), the textual data is associated with the surgical procedure; accessing a data-structure associating different portions of the surgical footage with different portions of the textual data (step 1206); presenting the textual data and a first part of the surgical footage to an individual, with a visual indication of a first part of the textual data (step 1208), the first part of the surgical footage is associated in the data-structure with the first part of the textual data; receiving from the individual a selection of a second part of the textual data (step 1210); using the data-structure to determine a second part of the surgical footage associated with the second part of the textual data (step 1212); presenting the second part of the surgical footage to the individual (step 1214); receiving from the individual a selection of a third part of the surgical footage (step 1216); using the data-structure to determine a third part of the textual data associated with the third part of the surgical footage (step 1218); and presenting a visual indication of the third part of the textual data to the individual (step 1220). In other examples, process 1200 may include additional steps or fewer steps. In other examples, one or more steps of process 1200 may be executed in a different order and/or one or more groups of steps may be executed simultaneously.

In some examples, a system for synchronized browsing of surgical videos and operative reports may include at least one processing unit configured to perform process 1200 and/or one or more of the steps of process 1200. In one example, the system may further comprise a digital communication device configured to receive an input of surgical footage of a surgical procedure (such as the surgical footage of step 1002, a surgical footage of a surgical procedure performed on a patient by a health care provider, a surgical footage of a different surgical procedure, and so forth), and/or to receive textual data in a natural language (such as the textual data of step 1204, textual data from an operative report, different textual data, and so forth). In one example, the system may further comprise at least one image sensor configured to capture surgical footage of a surgical procedure (such as the surgical footage of step 1002, a surgical footage of a surgical procedure performed on a patient by a health care provider, a surgical footage of a different surgical procedure, and so forth). In one example, the system may further comprise at least one digital memory unit configured to store surgical footage of a surgical procedure (such as the surgical footage of step 1002, a surgical footage of a surgical procedure performed on a patient by a health care provider, a surgical footage of a different surgical procedure, and so forth), and/or textual data in a natural language (such as the textual data of step 1204, textual data from an operative report, different textual data, and so forth). In one example, the system may further comprise at least one input device (such as a keyboard, a pointing device, a computer mice, a touchpad, a touchscreen, a microphone for receiving voice commands, a camera for detecting gestures, etc.) configured to receive selections for an individual (such as the selection of the second part of the textual data of step 1210, the selection of the third part of the surgical footage of step 1216, a different selection, and so forth). In one example, the system may further comprise at least one visual output device (such as a computer display screen, a projector, a head-mounted display, etc.) configured to present surgical footages and/or textual data and/or visual indications (for example, as described in step 1208 and/or step 1214 and/or step 1220). In some examples, a non-transitory computer readable medium may store computer implementable instructions that when executed by at least one processor may cause the at least one processor to perform operations for synchronized browsing of surgical videos and operative reports, and the operations may include process 1200 and/or one or more of the steps of process 1200. In some examples, a method for synchronized browsing of surgical videos and operative reports may include performing the steps of process 1200.

In some examples, each of the first part of the textual data (of step 1208), the second part of the textual data (of step 1210) and the third part of the textual data (of step 1218) may include at least one respective sentence in the natural language (or in other examples, at least one respective paragraph in the natural language, at least one word in the natural language, and so forth). In some examples, each of the first part of the textual data (of step 1208), the second part of the textual data (of step 1210) and the third part of the textual data (of step 1218) may include a respective first textual portion in the natural language automatically generated based on a respective analysis of a respective plurality of frames and a respective second textual portion in the natural language manually entered by the health care provider, may include text in the natural language automatically generated based on a respective analysis of a respective plurality of frames, may include text manually entered by the health care provider, and so forth. For example, the second part of the textual data (of step 1210) may include text in the natural language automatically generated based on an analysis of a plurality of frames of the surgical footage, and the third part of the textual data (of step 1218) may include text in the natural language manually entered by the health care provider.

In some examples, step 1204 may comprise receiving textual data in a natural language. The textual data may be associated with a surgical procedure (for example, with the surgical procedure of step 1002, with a different surgical procedure, and so forth). In one example, the textual data may be textual data created manually (for example, by the health care provider of step 1002, by a different person, etc.), may be textual data generated automatically (for example, as described above, and/or based on an analysis of the surgical footage received by step 1002, and/or using process 901, and/or using process 1100, and/or using process 1300, etc.), and so forth. In one example, step 1204 may read the textual data from a digital memory unit, may receive the textual data from an external computing device (for example, using a digital communication device), may generate the textual data automatically, and so forth. In some examples, the textual data received by step 1204 may be textual data from an operative report associated with the surgical procedure. For example, step 1204 may use step 1004 to receive the textual data.

In some examples, step 1206 may comprise accessing a data-structure associating different portions of a surgical footage (such as the surgical footage received by step 1002, a different surgical footage, etc.) with different portions of textual data (such as the textual data received by step 1204, different textual data, and so forth). In one example, the data-structure may create an injective correspondence between the portions of the surgical footage and the portions of the textual data (one non-limiting example of such data-structure may include a bidirectional map). In another example, the data-structure may create a non-injective correspondence between the portions of the surgical footage and the portions of the textual data (one non-limiting example of such data-structure may include two maps, one map that maps portions of the surgical footage to portions of the textual data, and another map that maps portions of the textual data to portions of the surgical footage). In one example, step 1206 may read the data-structure from a digital memory unit, may access a database storing the data-structure, may access and/or receive the data-structure via an external computing device (for example, using a digital communication device), may generate the data-structure automatically, and so forth. One non-limiting example of such data-structure may include data-structure 600, adding an additional column for the positions in the textual data corresponding to the different rows. In one example, a portion of the surgical footage depicting a surgical event may be associated in the data-structure accessed by step 1206 with a portion of the textual data indicative of the surgical event. In another example, a portion of the surgical footage depicting a surgical tool may be associated in the data-structure accessed by step 1206 with a portion of the textual data indicative of the surgical tool. In yet another example, a portion of the surgical footage depicting a surgical tool may be associated in the data-structure accessed by step 1206 with a portion of the textual data indicative of an alternative surgical tool. In some examples, a particular portion of the surgical footage may be associated in the data-structure accessed by step 1206 with a particular portion of the textual data inconsistence with the particular portion of the surgical footage. In one example, process 1200 may further comprise presenting the particular portion of the surgical footage to the individual, with a visual indication of the particular portion of the textual data, with a visual indication of inconsistency and/or a suggested modification to the particular portion of the textual data to correct the inconsistency. In one example, the suggested modification may be automatically generated, for example using step 1314. In another example, the suggested modification may be received from an external process (for example, via a digital communication device, via a memory unit, etc.) or from an individual (for example, via a user interface).

In some examples, a first portion of the surgical footage accessed by step 1002 may depict a first object, a second portion of the surgical footage accessed by step 1002 may depict a second object, a specific noun may be indicative of both the first and second objects, a first portion of the textual data received by step 1204 may include the specific noun adjacent to a first adjective, a second portion of the textual data received by step 1204 may include the specific noun adjacent to a second adjective, and the data-structure accessed by step 1206 may associate the first portion of the surgical footage with the first portion of the textual data based on the first adjective and a characteristic of the first object in the surgical footage, and/or may associate the second portion of the surgical footage with the second portion of the textual data based on the second adjective and a characteristic of the second object in the surgical footage. In some examples, a first portion of the surgical footage accessed by step 1002 may depict a first event, a second portion of the surgical footage accessed by step 1002 may depict a second event, a specific verb may be indicative of both the first and second events, a first portion of the textual data received by step 1204 may include the specific verb adjacent to a first adverb, a second portion of the textual data received by step 1204 may include the specific verb adjacent to a second adverb, and the data-structure accessed by step 1206 may associate the first portion of the surgical footage with the first portion of the textual data based on the first adverb and a characteristic of the first event in the surgical footage, and/or may associate the second portion of the surgical footage with the second portion of the textual data based on the second adverb and a characteristic of the second event in the surgical footage.

In some examples, step 1206 may analyze a plurality of frames of the surgical footage and the textual data to generate the data-structure. For example, step 1206 may use a multi-modal foundation model to analyze the plurality of frames and the textual data to generate the data-structure (for example with a suitable textual prompt, such as ‘find events that are depicted in the attached footage and described in the attached text, and create a table associating beginning and ending time-stamps of the depictions with the corresponding beginning and ending positions in of the description in the text’). In another example, step 1206 may analyze the plurality of frames using a visual event detection algorithm to identify events depicted in the surgical footage (and their locations in the surgical footage), may analyze the textual data using an NLP algorithm to identify descriptions corresponding to the events (and the positions of the descriptions in the textual data), and create the data-structure associating each identified event with the corresponding identified description. In one example, step 1206 may use a machine learning model to analyze the plurality of frames and the textual data to generate the data-structure. The machine learning model may be a machine learning model trained using training examples to create data-structure associating portions of videos with portions of text. An example of such training example may include a sample surgical video and sample textual data, together with a sample data-structure associating different portions of the sample surgical video with different portions of the sample textual data. In one example, step 1206 may use an artificial neural network to analyze the plurality of frames and the textual data to generate the data-structure. For example, the artificial neural network may be configured to identify correspondences between portions of input video and portion of textual input.

In some examples, step 1206 may identify a first mathematical object in a mathematical space, wherein the first mathematical object may correspond to at least part of a first portion of the textual data, for example as described above. Further, step 1206 may identify a second mathematical object in a mathematical space, wherein the second mathematical object may correspond to at least part of a second portion of the textual data, for example as described above. Further, step 1206 may calculate a convolution of at least part of a first portion of the surgical footage to obtain a first numerical value, and/or may calculate a convolution of at least part of a second portion of the surgical footage to obtain a second numerical value. Further, step 1206 may calculate a function of the first mathematical object and the first numerical value to obtain a third mathematical object in the mathematical space, and/or may calculate a function of the first mathematical object and the second numerical value to obtain a fourth mathematical object in the mathematical space, and/or may calculate a function of the second mathematical object and the second numerical value to obtain a fifth mathematical object in the mathematical space, for example as described above. In one example, step 1206 may, based on the third mathematical object, associate in the data-structure the first portion of the surgical footage with the first portion of the textual data. In one example, step 1206 may, based on the fourth mathematical object, avoid associating in the data-structure the second portion of the surgical footage with the first portion of the textual data. In one example, step 1206 may, based on the fifth mathematical object, associate in the data-structure the second portion of the surgical footage with the second portion of the textual data. For example, step 1206 may identify a specific mathematical object in a mathematical space, wherein the specific mathematical object may correspond to at least part of a specific portion of the textual data (for example as described above); may calculate a convolution of at least part of a specific portion of the surgical footage to obtain a specific numerical value; may calculate a function of the specific mathematical object and the specific numerical value to obtain a particular mathematical object in the mathematical space; and may associate in the data-structure the specific portion of the surgical footage with the specific portion of the textual data if and only if the particular mathematical object is in a specific region the mathematical space.

In some examples, a part of a surgical footage (such as a part of the surgical footage of step 1002, a part of a different surgical footage, etc.) may be presented. In some examples, textual data (such as the textual data received by step 1204, different textual data, etc.) may be presented, for example with a visual indication of a part of the textual data. In one example, the part of the surgical footage may be presented simultaneously with the presentation of the textual data and/or of the visual indication. In one example, the part of the surgical footage and the textual data with the visual indication may be presented in a same display device and/or using a same user interface. In another example, the part of the surgical footage may be presented in a different display device and/or using a different software application from the textual data with the visual indication. For example, step 1208 may comprise presenting the textual data and a first part of the surgical footage to an individual (for example, to the health care provider of step 1002, to a different individual, etc.), with a visual indication of a first part of the textual data. In one example, the first part of the surgical footage may be associated in the data-structure with the first part of the textual data. In one example, step 1214 may comprise presenting the second part of the surgical footage to the individual. In one example, step 1220 may comprise presenting a visual indication of the third part of the textual data to the individual. In one example, step 520 and/or process 500 and/or video playback region 410 and/or a video player may be used to present the part of the surgical footage. In one example, the part of surgical footage and/or the textual data and/or the visual indication may be presented using a display device, may be presented using a computer display screen, may be presented using a projector, may be presented using a head mounted display, may be presented in a user interface, and so forth. In some examples, the presentation of the textual data may enable an individual (such as the health care provider of step 1002, the individual of step 1208, a different individual, etc.) to modify the textual data.

In some examples, step 1210 may comprise receiving from an individual (such as, the individual of step 1208 and/or step 1214 and/or step 1216 and/or step 1220, a different individual, etc.) a selection of a second part of the textual data received by step 1204 (and/or a selection of a specific part of another textual data). For example, a user interface used for the presentation by step 1210 may enable the individual to select the second part of the textual data with a pointing device, for example using click and drag to mark the second part of the textual data, using selection of a specific location within the second part of the textual data (an automatic selection algorithm may use the specific location to determine the second part of the textual data, for example using a LLM to understand context, or using simple selection rules, such as a selection of a paragraph that includes the specific location), and so forth. In another example, a user interface used for the presentation by step 1210 may enable the individual to select the second part of the textual data with a keyboard, for example using the arrow keys to mark the second part of the textual data or to indicate the specific location within the second part of the textual data. In yet another example, a user interface used for the presentation by step 1210 may enable the individual to provide input in a natural language indicative of the second part of the textual data (such as, ‘the description of the ligation of the right gonadal vein’), and a LLM may be used to analyze the input in the natural language and the textual data to automatically select the second part of the textual data (for example with a suitable textual prompt, such as ‘which part of the attached textual data best match the attached description’). In one example, step 1210 and/or step 1220 may further comprise halting the presentation of the visual indication of the first part of the textual data. In one example, step 1210 may further comprise presenting a visual indication of the second part of the textual data. Further, step 1220 may halt the presentation of the visual indication of the second part of the textual data.

In some examples, step 1216 may comprise receiving from an individual (such as, the individual of step 1208 and/or step 1210 and/or step 1214 and/or step 1220, a different individual, etc.) a selection of a third part of the surgical footage accessed by step 1002 (and/or a selection of a specific part of a different surgical footage). For example, a user interface used for the presentation by step 1210 may enable the individual to select the third part of the surgical footage, for example using a seek bar control in a video player included in the user interface, using events markers (for example, as described above in relation to FIG. 4), using process 500 and/or step 540, and so forth. In another example, a user interface used for the presentation by step 1210 may enable the individual to provide input in a natural language indicative of the third part of the surgical footage (such as, ‘the ligation of the right gonadal vein’), and a multi-modal foundation model may be used to analyze the input in the natural language and the surgical footage to automatically select the third part of the surgical footage (for example with a suitable textual prompt, such as ‘which part of the attached footage best match the attached description’).

In some examples, step 1212 may comprise using a data-structure (such as the data-structure accessed by step 1206, a different data-structure, etc.) to determine a second part of the surgical footage associated with the second part of the textual data of step 1210 (and/or using the data-structure to determine a portion of surgical footage associated with the portion of textual data). For example, step 1212 may access the data-structure using an indication of the second part of the textual data (such as, a position of the second part of the textual data in the textual data, the text included in the second part of the textual data, etc.), for example as a key, to obtain an indication of the second part of the surgical footage (such as, a position of the second part of the surgical footage in the surgical footage, the frames included in the second part of the surgical footage, and so forth). In some examples, step 1218 may comprise using a data-structure (such as the data-structure accessed by step 1206, a different data-structure, etc.) to determine a third part of the textual data associated with the third part of the surgical footage of step 1216 (and/or using the data-structure to determine a portion of textual data associated with a portion of surgical footage). For example, step 1218 may access the data-structure using an indication of the third part of the surgical footage (such as, a position of the third part of the surgical footage in the surgical footage, the frames included in the third part of the surgical footage, etc.), for example as a key, to obtain an indication of the third part of the textual data (such as, a position of the third part of the textual data in the textual data, the text included in the third part of the textual data, and so forth).

In some examples, process 1200 may further comprise receiving from the individual a modification to the textual data received by step 1204. For example, a user interface used for the presentation by step 1210 may enable the individual to edit the textual data using a keyboard and/or a pointing device, for example in a text editor. In another example, a user interface used for the presentation by step 1210 may enable the individual to provide input in a natural language indicative of a desired to modify the textual data, and process 1200 may further comprise using a multi-modal foundation model to analyze the input in the natural language and the textual data to automatically modify the textual data (for example with a suitable textual prompt, such as ‘modify the attached textual data based on the attached instructions’). Further, process 1200 may further comprise analyzing a plurality of frames of the surgical footage and the modified textual data to update at least one association in the data-structure accessed by step 1206. For example, process 1200 may regenerate the data-structure automatically based on the modified textual data, for example as described above in relation to step 1206. In another example, process 1200 may identify the portions of the textual data modified, may remove associations corresponding to the modified portions from the data-structure, and may add new associations corresponding to the modified portions (for example, automatically as described above in relation to step 1206).

In some examples, process 1200 may further comprise receiving from the individual a selection of a fourth part of the surgical footage accessed by step 1002 (for example as described above in relation to step 1216 and the third part of the surgical footage); determining that the fourth part of the surgical footage is not associated with any portion of the textual data received by step 1204 in the data-structure accessed by step 1206 (for example, by accessing the data-structure with a key based on the fourth part of the surgical footage); and providing an indication of the lack of association (such as a visual indication, a graphical indication, a textual indication, an audible indication, an indication in a natural language, and so forth). In some examples, process 1200 may further comprise receiving from the individual a selection of a fourth part of the textual data received by step 1204 (for example as described above in relation to step 1210 and the second part of the textual data); determining that the fourth part of the textual data is not associated with any portion of the surgical footage in the data-structure accessed by step 1206 (for example, by accessing the data-structure with a key based on the fourth part of the textual data); and providing an indication of the lack of association (such as a visual indication, a graphical indication, a textual indication, an audible indication, an indication in a natural language, and so forth).

In some examples, process 1200 may further comprise presenting a plurality of issues associated with the textual data to the individual (for example, using step 1308, using a user interface, etc.); receiving from the individual a selection of a particular issue of the plurality of issues (for example, using step 1312, using a user interface, using a voice command, using a gesture, using a pointing device, using a keyboard, using a touchscreen, using a touchpad, etc.); and in in response to the selection of the particular issue, presenting to the individual a portion of the surgical footage associated with the particular issue and a visual indication of a portion of the textual data associated with the particular issue (for example, using user interface 1240, using a different user interface, as described above in relation to step 1208, and so forth).

In some examples, process 1200 may further comprise, while presenting the first part of the surgical footage (of step 1208) and the visual indication of the first part of the textual data (of step 1208), receiving an input from the individual for correcting the association in the data-structure accessed by step 1206 of the first part of the surgical footage with the first part of the textual data; and updating the data-structure based on the input from the individual. For example, such correction may include one or more of removing the association, associating the first part of the surgical footage with a different portion of the textual data, associating the first part of the textual data with a different portion of the surgical footage, and so forth. For example, the input from the individual may be received using user interface 1240, using a different user interface, using a voice command, using a gesture, using a pointing device, using a keyboard, using a touchscreen, using a touchpad, and so forth.

In some examples, process 1200 may further comprise providing a user interface control to enable the individual to select a mode of presentation from at least a first mode of presentation and a second mode of presentation. In one example, in the first mode of presentation, a selection of a segment of the surgical footage automatically causes a presentation of a visual indication of a segment of the textual data associated with the selected segment of the surgical footage, and in the second mode of presentation, the selection of the segment of the surgical footage does not cause an automatic presentation of the visual indication of the segment of the textual data associated with the selected segment of the surgical footage. In another example, in the first mode of presentation, a selection of a segment of the textual data automatically causes a presentation of a segment of the surgical footage associated with the selected segment of the textual data, and in the second mode of presentation, the selection of the segment of the textual data does not cause an automatic presentation of the segment of the surgical footage associated with the selected segment of the textual data.

In some examples, process 1200 may further comprise presenting a playback of the surgical footage received by step 1002; and while the playback is in different portions of the surgical footage, presenting corresponding visual indications of the corresponding portions of the textual data based on the data-structure accessed by step 1206. For example, while the playback is in a first portion of the surgical footage, process 1200 may present a visual indication of a first portion of the textual data, the first portion of the surgical footage may be associated in the data-structure with the first portion of the textual data, and/or while the playback is in a second portion of the surgical footage, process 1200 may present a visual indication of a second portion of the textual data, the second portion of the surgical footage may be associated in the data-structure with the second portion of the textual data.

FIG. 12B is a block diagram illustrating an example user interface 1240, consistent with some embodiments of the present disclosure. In this example, user interface 1240 may comprise a video playback region 1242 (for example, as described above in relation to video playback region 410) and a presentation of textual data 1250. In one example, the video playback region 1242 may be associated with a control bar 1244 (for example, as described above in relation to control bar 412), a timeline 1246 (for example, as described above in relation to timeline 420), a video position indicator 1248 (for example, as described above in relation to position indicator 424), and so forth. In one example, the presentation of textual data 1250 may be associated with a range slider 1252, a text position indicator 1254, and so forth. In this example, range slider 1252 may enable an individual to display different portions of textual data 1250 when the textual data is too long to be displayed entirely in the presentation area, and text position indicator 1254 may indicate the portion of the textual data being presented at the moment. In one example, a visual indication of a portion of the textual data presented in 1250 may be displayed. In the example of FIG. 12B, the portion of the textual data presented in 1250 is displayed using different font properties (in this example, bold vs regular and a larger font size). Some non-limiting examples of such visual indication may include display using different font and/or different font properties, display using different font color, display over different background color and/or texture, display of an arrow or other visual symbol indicating the portion, a box or another shape surrounding the portion, or any other text highlighting method. In some examples, step 1208 may present the textual data received by step 1204 in 1250, may present the first part of the surgical footage received by step 1002 in video playback region 1242 (for example by setting the playback location to a frame included in the first part), and may present a visual indication of the first part of the textual data as described above. Further, step 1214 may present the second part of the surgical footage in video playback region 1242, for example by setting the playback location to a frame included in the second part. Further, step 1220 may present a visual indication of the third part of the textual data as described above. In some examples, step 1210 may use user interface 1240 to receive from the individual the selection of a second part of the textual data (for example, by selecting portion of the textual data in 1250), and/or step 1216 may use user interface 1240 to receive from the individual the selection of a third part of the surgical footage (for example, by repositioning video position indicator 1248).

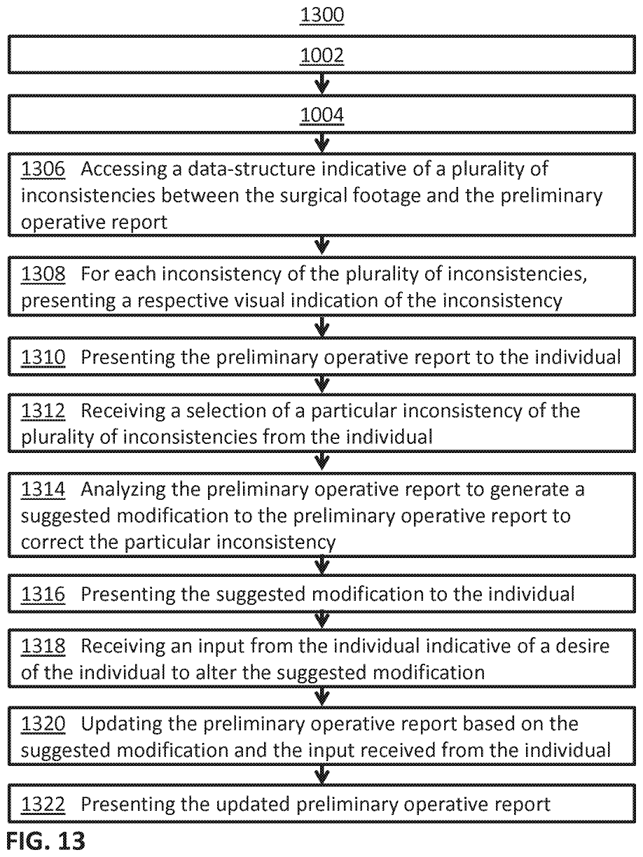

FIG. 13 is a flow diagram of an exemplary process 1300 for assisted resolution of inconsistencies between surgical footages and operative reports, consistent with some embodiments of the present disclosure. In this example, process 1300 may comprise receiving an input of surgical footage of a surgical procedure performed on a patient by a health care provider (step 1002); receiving a preliminary operative report associated with the surgical procedure (step 1004); accessing a data-structure indicative of a plurality of inconsistencies between the surgical footage and the preliminary operative report (step 1306); for each inconsistency of the plurality of inconsistencies, presenting a respective visual indication of the inconsistency to an individual (step 1308); presenting the preliminary operative report to the individual (step 1310); receiving a selection of a particular inconsistency of the plurality of inconsistencies from the individual (step 1312); analyzing the preliminary operative report to generate a suggested modification to the preliminary operative report to correct the particular inconsistency (step 1314); presenting the suggested modification to the individual (step 1316); receiving an input from the individual indicative of a desire of the individual to alter the suggested modification (step 1318); updating the preliminary operative report based on the suggested modification and the input received from the individual (step 1320); and presenting the updated preliminary operative report to the individual (step 1322). In other examples, process 1300 may include additional steps or fewer steps. For example, steps 1318, 1320 and/or 1322 may be excluded from process 1300. In other examples, one or more steps of process 1300 may be executed in a different order and/or one or more groups of steps may be executed simultaneously.

In some examples, a system for assisted resolution of inconsistencies between surgical footages and operative reports may include at least one processing unit configured to perform process 1300 and/or one or more of the steps of process 1300. In one example, the system may further comprise a digital communication device configured to receive an input of surgical footage of a surgical procedure (such as the surgical footage of step 1002, a surgical footage of a surgical procedure performed on a patient by a health care provider, a surgical footage of a different surgical procedure, and so forth), and/or to receive a preliminary operative report associated with a surgical procedure (such as the preliminary operative report of step 1004, a different preliminary operative report, etc.), and/or to receive the selection of the particular inconsistency of step 1312 (for example, from a personal computing device of an individual, from a cloud platform, etc.), and/or to receive the input of step 1318 (for example, from a personal computing device of an individual, from a cloud platform, etc.), and so forth. In one example, the system may further comprise at least one image sensor configured to capture surgical footage of a surgical procedure (such as the surgical footage of step 1002, a surgical footage of a surgical procedure performed on a patient by a health care provider, a surgical footage of a different surgical procedure, and so forth). In one example, the system may further comprise at least one digital memory unit configured to store surgical footage of a surgical procedure (such as the surgical footage of step 1002, a surgical footage of a surgical procedure performed on a patient by a health care provider, a surgical footage of a different surgical procedure, and so forth), and/or a preliminary operative report associated with a surgical procedure (such as the preliminary operative report of step 1004, a different preliminary operative report, etc.), and/or the selection of the particular inconsistency of step 1312, and/or the input of step 1318. Further, the at least one processing unit may be configured to read from the at least one digital memory unit the surgical footage, and/or the preliminary operative report from the at least one digital memory unit, and/or the selection of the particular inconsistency of step 1312, and/or the input of step 1318. In one example, the system may further comprise at least one input device (such as a keyboard, a pointing device, a computer mice, a touchpad, a touchscreen, a microphone for receiving voice commands, a camera for detecting gestures, etc.) configured to receive selections for an individual (such as the selection of the particular inconsistency of step 1312, a different selection, and so forth), and/or to receive inputs from an individual (such as the input of step 1318, a different input, and so forth). In one example, the system may further comprise at least one visual output device (such as a computer display screen, a projector, a head-mounted display, etc.) configured to present surgical footages (such as the surgical footage received by step 1002, a different surgical footage, etc.), and/or to present a preliminary operative report (for example, as described in relation to step 1310 and/or step 1322), and/or to present visual indications of inconsistencies (for example, as described in relation to step 1308), and/or to present suggested modification (for example, as described in relation to step 1316). In some examples, a non-transitory computer readable medium may store computer implementable instructions that when executed by at least one processor may cause the at least one processor to perform operations for assisted resolution of inconsistencies between surgical footages and operative reports, and the operations may include process 1300 and/or one or more of the steps of process 1300. In some examples, a method for assisted resolution of inconsistencies between surgical footages and operative reports may include performing the steps of process 1300.

In some examples, step 1306 may comprise accessing a data-structure indicative of a plurality of inconsistencies between surgical footage of a surgical procedure (such as the surgical footage received by step 1002, a surgical footage of a surgical procedure performed on a patient by a health care provider, a surgical footage of a different surgical procedure, and so forth) and a preliminary operative report associated with the surgical procedure (such as the preliminary operative report of step 1004, a different preliminary operative report, and so forth). For example, the data-structure may be a software container of inconsistencies including the plurality of inconsistencies. In another example, the data-structure may be a database containing the plurality of inconsistencies. In one example, step 1306 may read the data-structure from a digital memory unit, may access a database storing the data-structure, may receive the data-structure from an external computing device (for example, using a digital communication device), may generate the data-structure automatically, and so forth. In some examples, step 1306 may analyze the preliminary operative report and the surgical footage to identify the plurality of inconsistencies, for example using step using step 1006. Further, step 1306 may generate the data-structure from the identified plurality of inconsistencies, and/or may add the identified plurality of inconsistencies to an existing data-structure.

In some examples, step 1308 may comprise presenting an indication (such as a visual indication, an audible indication, a textual indication, a graphical indication, etc.) of an inconsistency between a surgical footage and a preliminary operative report (such as an inconsistency of the plurality of inconsistencies of step 1306, a different inconsistency, etc.), for example to an individual. In one example, step 1308 may comprise, for each inconsistency of the plurality of inconsistencies of step 1306, presenting a respective indication (such as a respective visual indication, a respective audible indication, a respective textual indication, a respective graphical indication, etc.) of the inconsistency to an individual. For example, step 1308 may present the visual indicators via a user interface. In another example, step 1308 may present the visual indicators using at least one visual output device (such as a computer display screen, a projector, a head-mounted display, and so forth). In yet another example, a visual indicator presented by step 1308 may be a graphical indication, may be a textual indication, and so forth. In one example, step 1308 may, for each inconsistency of the plurality of inconsistencies of step 1306, use step 1008 to present the respective indication of the inconsistency. In one example, a visual indication of a specific inconsistency (such as the visual indication of a specific inconsistency of the plurality of inconsistencies presented by step 1308) may include a visual indication of a portion of the preliminary operative report associated with the specific inconsistency and/or a visual presentation of a portion of the surgical footage associated with the specific inconsistency, for example as described above in relation to step 1008 and/or step 1208.

In some examples, step 1310 may comprise presenting a preliminary operative report (such as operative report 701, the preliminary operative report received by step 1004, a different preliminary operative report, etc.) to an individual (such as the individual of step 1308, a different individual, etc.), for example as described above. In some examples, step 1322 may comprise presenting an updated preliminary operative report (such as the updated preliminary operative report generated by step 1320, a different updated preliminary operative report, etc.) to an individual (such as the individual of step 1308 and/or step 1310 and/or step 1312 and/or step 1316 and/or step 1318, a different individual, and so forth). In some examples, step 1310 and/or step 1322 may present the preliminary operative report and/or the updated preliminary operative report visually, may present the preliminary operative report and/or the updated preliminary operative report audibly, may present the preliminary operative report and/or the updated preliminary operative report via a user interface (such as user interface 1240, the user interface used by step 1308, a text editor, a different user interface, etc.), and so forth.

In some examples, step 1312 may comprise receiving a selection of a particular inconsistency (for example a selection of a particular inconsistency of the plurality of inconsistencies of step 1306), for example from an individual (such as the individual of step 1308 and/or step 1310, a different individual, and so forth). For example, a user interface (such as the user interface used by step 1308 and/or step 1310, a different user interface, etc.) may enable the individual to select the particular inconsistency (for example, using a pointing device, using a keyboard, using a touchpad, using a touchscreen, using gestures, using voice commands, etc.), and step 1312 may receive the selection of the particular inconsistency via the user interface. In one example, step 1312 may read an indication of the selection of the particular inconsistency from a digital memory unit, may receive an indication of the selection of the particular inconsistency from an external computing device (for example, using a digital communication device), may receive the selection of the particular inconsistency from a different process, and so forth.

In some examples, step 1314 may comprise analyzing a preliminary operative report (such as the preliminary operative report received by step 1004, a different preliminary operative report, etc.) and/or a surgical footage (such as the surgical footage received by step 1002, a different surgical footage, etc.) to generate a suggested modification to the preliminary operative report to correct a particular inconsistency (such as the particular inconsistency of step 1312, a different inconsistency, and so forth). In one example, step 1314 may use a trained machine learning model to analyze the preliminary operative report and/or the surgical footage and/or additional information to generate the suggested modification to the preliminary operative report to correct the particular inconsistency. The trained machine learning model may be a machine learning model trained using training examples to generate modifications to preliminary operative reports to correct inconsistencies based on the preliminary operative reports and/or surgical footages and/or additional information. An example of such training examples may include a sample preliminary operative report, an indication of a sample inconsistency, optionally a sample surgical video, and optionally additional information, together with a sample modification to the sample preliminary operative report to correct the sample inconsistency. In some examples, step 1314 may use an artificial neural network to analyze the preliminary operative report and/or the surgical footage to generate the suggested modification to the preliminary operative report to correct the particular inconsistency. For example, the artificial neural network may be configured to generate modifications to preliminary operative reports to correct inconsistencies based on the preliminary operative reports and/or surgical footages and/or additional information. In some examples, step 1314 may use a multi-modal foundation model to analyze the preliminary operative report and/or the surgical footage to generate the suggested modification to the preliminary operative report to correct the particular inconsistency (for example with a suitable textual prompt, such as ‘suggest a modification to the attached operative report to correct the inconsistency {textual description of the particular inconsistency} with the attached surgical video’).

In some examples, step 1314 may identify a first mathematical object in a mathematical space, wherein the first mathematical object corresponds to at least part of the preliminary operative report (for example, at least part of a textual portion the preliminary operative report, at least part of a numerical portion of the preliminary operative report, at least one code indicated in the preliminary operative report, etc.), for example as described above. Further, step 1314 may calculate a convolution of at least part of the surgical footage to obtain a numerical value. Further, step 1314 may calculate a function of the first mathematical object and the numerical value to obtain a second mathematical object in the mathematical space, for example as described above. Further, step 1314 may base the generation of the suggested modification to the preliminary operative report on the second mathematical object. For example, when the second mathematical object is in a selected group of mathematical objects (for example, in a selected region of the mathematical space), step 1314 may include one modification in the generated suggested modification, and when the second mathematical object is not in the selected group of mathematical objects (for example, outside the selected region of the mathematical space), step 1314 may include a different modification in the generated suggested modification.

In some examples, the suggested modification generated by step 1314 may include an automatically generated text in a natural language and/or a position in the preliminary operative report for an insertion of the automatically generated text. For example, step 1314 may analyze the preliminary operative report and/or the surgical footage to generate the automatically generated text and/or to determine the position in the preliminary operative report for the insertion of the automatically generated text. In one example, step 1314 may use a LLM to generate the automatically generated text and/or to determine the position in the preliminary operative report for the insertion of the automatically generated text (for example with a suitable textual prompt, such as ‘generate a textual description of the ligation of the right gonadal vein in the attached surgical footage’, or ‘generate a textual description adding details not included in the attached operative report on the ligation of the right gonadal vein in the attached surgical footage, and identify a position in the attached operative report an insertion of the textual description’). In one example, the automatically generated text may identify an event depicted in the surgical footage that is not reflected in the preliminary operative report, and/or may indicate a characteristic of the event determined by analyzing the surgical footage. In another example, the automatically generated text may identify a surgical tool depicted in the surgical footage that is not reflected in the preliminary operative report, and/or may indicate a characteristic of the surgical tool determined by analyzing the surgical footage. In yet another example, the automatically generated text may indicate a condition of an anatomical structure depicted in the surgical footage.

In some examples, the suggested modification generated by step 1314 may include a removal of an automatically selected portion of the preliminary operative report. For example, step 1314 may analyze the preliminary operative report and/or the surgical footage to select the automatically selected portion. In one example, step 1314 may use a LLM to select the automatically selected portion (for example with a suitable textual prompt, such as ‘identify the portion of the attached operative report describing a ligation of the right gonadal vein’, or ‘identify the portion of the attached operative report describing an event that is not reflected in the attached surgical footage’). In one example, the automatically selected portion may indicate an event that is not depicted in the surgical footage. In another example, the automatically selected portion may indicate a surgical tool that is not depicted in the surgical footage.

In some examples, the suggested modification generated by step 1314 may include a modification of an automatically selected portion of a text in a natural language included in the preliminary operative report. For example, step 1314 may analyze the preliminary operative report and/or the surgical footage to select the automatically selected portion and/or to determine the modification to the automatically selected portion. In one example, step 1314 may use a LLM to select the automatically selected portion to determine the modification to the automatically selected portion (for example with a suitable textual prompt, such as ‘identify the portion of the attached operative report describing a ligation of the right gonadal vein, and suggest a modification to the identified portion reflecting that the vein was ligated using a bipolar electrocautery rather than suturing’, or ‘identify the portion of the attached operative report including a characteristic of an event that is different from a characteristic of the event in the attached surgical footage, and suggest a modification to the identified portion to correct the characteristic of the event to be the consistence with the attached surgical footage’). In one example, the modification of the automatically selected portion of the text may include at least one of a change of a characteristic of an event to a characteristic determined based on an analysis of the surgical footage, a change of a characteristic of a surgical tool to a characteristic determined based on an analysis of the surgical footage, or a change of a characteristic of an anatomical structure to a characteristic determined based on an analysis of the surgical footage.

In some examples, the particular inconsistency of step 1312 may include an event depicted in the surgical footage received by step 1002 that is not reflected in the preliminary operative report received by step 1004. Further, the suggested modification generated by step 1314 may include an automatically generated text in a natural language indicating the event and/or a position in the preliminary operative report for an insertion of the automatically generated text. In some examples, the particular inconsistency of step 1312 may include a surgical tool depicted in the surgical footage received by step 1002 that is not reflected in the preliminary operative report received by step 1004. Further, the suggested modification generated by step 1314 may include an automatically generated text in a natural language indicating the surgical tool and/or a position in the preliminary operative report for an insertion of the automatically generated text. In some examples, the particular inconsistency of step 1312 may include an inconsistency in a quantity of surgical staples and/or an inconsistency in a color associated with surgical staples. Further, the suggested modification generated by step 1314 may include a replacement of at least one word in a natural language indicative of a quantity and/or of a color. In some examples, the particular inconsistency of step 1312 may include a secondary surgical trauma depicted in the surgical footage received by step 1002 but not reflected in the preliminary operative report received by step 1004. Further, the suggested modification generated by step 1314 may include an automatically generated text in a natural language indicating the secondary surgical trauma and/or a position in the preliminary operative report for an insertion of the automatically generated text. In some examples, the particular inconsistency of step 1312 may include an anatomical structure depicted in the surgical footage received by step 1002 in a condition that is not reflected in the preliminary operative report received by step 1004. Further, the suggested modification generated by step 1314 may include an automatically generated text in a natural language indicating the condition and/or a position in the preliminary operative report for an insertion of the automatically generated text. In some examples, the particular inconsistency of step 1312 may include an anatomical structure depicted in the surgical footage received by step 1002 in a size that is different from a size of the anatomical structure reflected in the preliminary operative report received by step 1004. Further, the suggested modification generated by step 1314 may include a replacement and/or an addition of at least one word in a natural language indicative of size. In some examples, the particular inconsistency of step 1312 may include an event indicated in the preliminary operative report received by step 1004 that is not depicted in the surgical footage received by step 1002. Further, the suggested modification generated by step 1314 may include a removal of an automatically selected portion of the preliminary operative report indicative of the event. In some examples, the particular inconsistency of step 1312 may include a surgical tool indicated in the preliminary operative report received by step 1004 that is not depicted in the surgical footage received by step 1002. Further, the suggested modification generated by step 1314 may include a removal of an automatically selected portion of the preliminary operative report indicative of the surgical tool.

In some examples, the particular inconsistency of step 1312 may be based on a noun and an adjective adjacent to the noun in the preliminary operative report received by step 1004. In one example, the noun may be indicative of an element in the surgical footage received by step 1002, and the particular inconsistency may include a characteristic of the element in the surgical footage being inconsistence with the adjective. Further, the suggested modification generated by step 1314 may include a replacement of the adjective with a different adjective selected based on an analysis of the surgical footage. In some examples, the particular inconsistency of step 1312 may be based on a verb and an adverb adjacent to the verb in the preliminary operative report received by step 1004. In one example, the verb may be indicative of a surgical action in the surgical footage received by step 1002, and the particular inconsistency may include a characteristic of the surgical action in the surgical footage being inconsistence with the adverb. Further, the suggested modification generated by step 1314 may include a replacement of the adverb with a different adverb selected based on an analysis of the surgical footage. In one example, the characteristic of the surgical action may be an extent of the surgical action, may be a timing of the surgical action may be a type of the surgical action, and so forth.

In some examples, the particular inconsistency of step 1312 may be based on an order of events in the surgical footage received by step 1002 being different from an order of the events indicated in the preliminary operative report received by step 1004. Further, the suggested modification generated by step 1314 may include a modification to the preliminary operative report to reflect the order of the events in the surgical footage. In some examples, the particular inconsistency of step 1312 may be based on a timing of an event in the surgical footage received by step 1002 being different from a timing of the event indicated in the preliminary operative report received by step 1004. Further, the suggested modification generated by step 1314 may include a modification to the preliminary operative report to reflect the timing of the event in the surgical footage. In some examples, the particular inconsistency of step 1312 may include an inconsistency in a duration of a phase of the surgical procedure. Further, the suggested modification generated by step 1314 may include a modification to the preliminary operative report to reflect the duration of the phase in the surgical footage received by step 1002. In some examples, the particular inconsistency of step 1312 may include an inconsistency in a CWT of a colonoscopy. Further, the suggested modification generated by step 1314 may include a modification to the preliminary operative report to reflect the CWT in the surgical footage received by step 1002.

In some examples, the particular inconsistency of step 1312 may include an inconsistency in a surgical technique. Some non-limiting examples of such surgical technique may include laparoscopic surgery, robotic surgery, open surgery, and so forth. Further, the suggested modification generated by step 1314 may include a modification to the preliminary operative report received by step 1004 to indicate the surgical technique corresponding to the surgical footage. In some examples, the preliminary operative report received by step 1004 may indicate at least one type of procedure. Further, the particular inconsistency of step 1312 may include a particular type of procedure consistence with the surgical footage received by step 1002 but not indicated by the preliminary operative report. Further, the suggested modification generated by step 1314 may include adding an indication of the particular type of procedure to the preliminary operative report. For example, the preliminary operative report may indicate ‘Robotic-assisted Radical Prostatectomy’ but not ‘Robotic-assisted Inguinal Hernia Repair’, while the surgical footage may be consistence with both, and the suggested modification may add an indication of ‘Robotic-assisted Inguinal Hernia Repair’ to the preliminary operative report. In some examples, the particular inconsistency of step 1312 may include a particular type of procedure indicated by the preliminary operative report received by step 1004 but inconsistence with the surgical footage received by step 1002. Further, the suggested modification generated by step 1314 may include removing the indication of the particular type of procedure from the preliminary operative report. For example, the preliminary operative report may indicate ‘Robotic-assisted Radical Prostatectomy’ and ‘Robotic-assisted Inguinal Hernia Repair’, while the surgical footage may be consistence only with ‘Robotic-assisted Radical Prostatectomy’, and the suggested modification may remove the indication of ‘Robotic-assisted Inguinal Hernia Repair’ from the preliminary operative report.

In some examples, step 1316 may comprise presenting a suggested modification (such as the suggested modification generated by step 1314, a suggested modification to the preliminary operative report received by step 1004, a suggested modification to a different preliminary operative report, etc.) to an individual (such as the individual of step 1308 and/or step 1310 and/or step 1312, a different individual, and so forth). In one example, step 1316 may present the suggested modification visually, may presented the suggested modification audibly, may present the suggested modification textually, may present the suggested modification graphically, may present the suggested modification via a user interface (such as user interface 1240, the user interface used by step 1308 and/or step 1310, a different user interface, etc.), and so forth. In one example, step 1316 may provide a natural language description of the suggested modification (the description may include at least one word and/or at least one phrase not included in the suggested modification and/or in the preliminary operative report). In one example, step 1316 may comprise presenting a first version of the preliminary operative report updated based on the suggested modification (for example as described above in relation to the preliminary operative report and/or step 1310), for example with a visual indication of the suggested modification (for example, in a ‘Track Changes’format). In some examples, the presenting the suggested modification to the individual by step 1316 may include presenting to the individual a modified version of the preliminary operative report including a modified portion of the preliminary operative report based on the suggested modification, for example with a visual indication of the modified portion.

In some examples, step 1318 may comprise receiving an input from an individual (such as the individual of step 1308 and/or step 1310 and/or step 1312 and/or step 1316, a different individual, and so forth) indicative of a desire of the individual to alter a suggested modification (for example, a suggested modification to a preliminary operative report, the suggested modification generated by step 1314, the suggested modification presented by step 1316, a different suggested modification, and so forth). For example, a user interface used for the presentation of the suggested modification by step 1316 may enable the individual to alter the suggested modification (and/or other portions of the preliminary operative report) using a keyboard and/or a pointing device, for example in a text editor. In another example, a user interface used for the presentation of the suggested modification by step 1316 may enable the individual to provide an input in a natural language indicative of a desired to alter the suggested modification (and/or other portions of the preliminary operative report), and step 1318 may receive from the individual an input in a natural language indicative of a desired to alter the suggested modification via the user interface.

In some examples, step 1320 may comprise updating a preliminary operative report (such as the preliminary operative report received by step 1004, a different preliminary operative report, etc.) based on a suggested modification (for example, a suggested modification to the preliminary operative report, the suggested modification generated by step 1314, a different suggested modification, etc.) and/or an input (such as an input received from an individual, the input from the individual received by step 1318, and so forth). For example, step 1316 may present a first version of the preliminary operative report updated based on the suggested modification (for example as described above) in a text editor, step 1318 may receive inputs from a keyboard and/or a pointing device for altering the first version of the preliminary operative report updated based on the suggested modification, and thereby step 1320 may further update the first updated version of the preliminary operative report. In another example, step 1318 may receive from the individual an input in a natural language indicative of a desired to alter the suggested modification (for example as described above), and step 1320 may use a LLM model to analyze the input in the natural language and/or the preliminary operative report and/or the suggested modification to update the preliminary operative report based on the suggested modification and the input in the natural language (for example with a suitable textual prompt, such as ‘modify the attached preliminary operative report based on the attached suggested modification and the attached instructions received in response to the suggested modification’).

In some examples, the input received from the individual by step 1318 may be an input in a natural language. Further, step 1320 may comprise analyzing the input received from the individual to determine at least one phrase for insertion, for example as described above. In one example, the at least one phrase may not be included in any one of the input received from the individual, the preliminary operative report and the suggested modification. Further, the updating the preliminary operative report by step 1320 may include inserting the at least one phrase. In some examples, the input received from the individual by step 1318 may be an input in a natural language. Further, step 1320 may analyze a plurality of frames of the surgical footage received by step 1002 based on the input received from the individual to determine at least one phrase for insertion. In one example, the at least one phrase may not be included in any one of the input received from the individual, the preliminary operative report and the suggested modification. Further, step 1320 may insert the at least one phrase into the preliminary operative report.

In some examples, the particular inconsistency of step 1312 may be an inconsistency based on the preliminary operative report, the surgical footage and a textual medical history of the patient (for example, a textual medical history of the patient in a natural language). In one example, the visual indication of the particular inconsistency presented by step 1308 may include a visual indication of information from the textual medical history associated with the particular inconsistency. Further, step 1314 may comprise analyzing the preliminary operative report and/or the textual medical history and/or the surgical footage to generate the suggested modification to the preliminary operative report to correct the particular inconsistency. For example, step 1314 may use at least part of the textual medical history as the additional information in the machine learning model described above in relation to step 1314. In another example, step 1314 may use at least part of the textual medical history as the additional information in the artificial neural network described above in relation to step 1314.

In some examples, the particular inconsistency of step 1312 may be an inconsistency based on the preliminary operative report, the surgical footage and an historic medical image of the patient. In one example, the visual indication of the particular inconsistency presented by step 1308 may include a visual indication of at least a portion of the historic medical image associated with the particular inconsistency. Further, step 1314 may comprise analyzing the preliminary operative report and/or the historic medical image and/or the surgical footage to generate the suggested modification to the preliminary operative report to correct the particular inconsistency. For example, step 1314 may use at least part of the historic medical image as the additional information in the machine learning model described above in relation to step 1314. In another example, step 1314 may use at least part of the historic medical image as the additional information in the artificial neural network described above in relation to step 1314.

In some examples, the particular inconsistency of step 1312 may be an inconsistency based on the preliminary operative report, the surgical footage and audio data recorded during the surgical procedure. Further, step 1314 may comprise analyzing the preliminary operative report and/or the audio data and/or the surgical footage to generate the suggested modification to the preliminary operative report to correct the particular inconsistency. For example, step 1314 may use at least part of the audio data as the additional information in the machine learning model described above in relation to step 1314. In another example, step 1314 may use at least part of the audio data as the additional information in the artificial neural network described above in relation to step 1314. In one example, the audio data may include speech in a natural language produced by the health care provider, and the suggested modification may be based on the speech. In another example, the audio data may include a conversation between the health care provider and another person, and the suggested modification may be based on the conversation.

What is claimed is:

1. A non-transitory computer readable medium containing instructions that when executed by at least one processor cause the at least one processor to perform operations for assisted resolution of inconsistencies between surgical footages and operative reports, the operations comprising:
receiving an input of surgical footage of a surgical procedure performed on a patient by a health care provider;
receiving a preliminary operative report associated with the surgical procedure;
accessing a data-structure indicative of a plurality of inconsistencies between the surgical footage and the preliminary operative report;
for each inconsistency of the plurality of inconsistencies, presenting a respective visual indication of the inconsistency to an individual;
presenting the preliminary operative report to the individual;
receiving a selection of a particular inconsistency of the plurality of inconsistencies from the individual;
analyzing the preliminary operative report to generate a suggested modification to the preliminary operative report to correct the particular inconsistency;
presenting the suggested modification to the individual;
receiving an input from the individual indicative of a desire of the individual to alter the suggested modification;
updating the preliminary operative report based on the suggested modification and the input received from the individual; and
presenting the updated preliminary operative report to the individual.

2. The non-transitory computer readable medium of claim 1, wherein the operations further comprise analyzing the preliminary operative report and the surgical footage to identify the plurality of inconsistencies.

3. The non-transitory computer readable medium of claim 1, wherein the operations further comprise:
identifying a first mathematical object in a mathematical space, the first mathematical object corresponds to at least part of the preliminary operative report;
calculating a convolution of at least part of the surgical footage to obtain a numerical value;
calculating a function of the first mathematical object and the numerical value to obtain a second mathematical object in the mathematical space; and
basing the generation of the suggested modification to the preliminary operative report on the second mathematical object.

4. The non-transitory computer readable medium of claim 1, wherein the particular inconsistency is based on the preliminary operative report, the surgical footage and a textual medical history of the patient, and the operations further comprise analyzing the preliminary operative report and the textual medical history to generate the suggested modification to the preliminary operative report to correct the particular inconsistency.

5. The non-transitory computer readable medium of claim 1, wherein the particular inconsistency is based on the preliminary operative report, the surgical footage and audio data recorded during the surgical procedure, and the operations further comprise analyzing the preliminary operative report and the audio data to generate the suggested modification to the preliminary operative report to correct the particular inconsistency.

6. The non-transitory computer readable medium of claim 1, wherein the particular inconsistency includes an event depicted in the surgical footage that is not reflected in the preliminary operative report, and the suggested modification includes an automatically generated text in a natural language indicating the event and a position in the preliminary operative report for an insertion of the automatically generated text.

7. The non-transitory computer readable medium of claim 1, wherein the particular inconsistency includes a surgical tool depicted in the surgical footage that is not reflected in the preliminary operative report, and the suggested modification includes an automatically generated text in a natural language indicating the surgical tool and a position in the preliminary operative report for an insertion of the automatically generated text.

8. The non-transitory computer readable medium of claim 1, wherein the particular inconsistency includes at least one of inconsistency in a quantity of surgical staples or inconsistency in a color associated with surgical staples, and wherein the suggested modification includes a replacement of at least one word in a natural language indicative of at least one of a quantity or a color.

9. The non-transitory computer readable medium of claim 1, wherein the particular inconsistency includes a secondary surgical trauma depicted in the surgical footage but not reflected in the preliminary operative report, and the suggested modification includes an automatically generated text in a natural language indicating the secondary surgical trauma and a position in the preliminary operative report for an insertion of the automatically generated text.

10. The non-transitory computer readable medium of claim 1, wherein the particular inconsistency includes an anatomical structure depicted in the surgical footage in a condition that is not reflected in the preliminary operative report, and the suggested modification includes an automatically generated text in a natural language indicating the condition.

11. The non-transitory computer readable medium of claim 1, wherein the particular inconsistency includes an anatomical structure depicted in the surgical footage in a size that is different from a size of the anatomical structure reflected in the preliminary operative report, and the suggested modification includes a replacement of at least one word in a natural language indicative of size.

12. The non-transitory computer readable medium of claim 1, wherein the particular inconsistency includes an event that is not depicted in the surgical footage, and the suggested modification includes a removal of an automatically selected portion of the preliminary operative report indicative of the event.

13. The non-transitory computer readable medium of claim 1, wherein the particular inconsistency is based on a noun and an adjective adjacent to the noun in the preliminary operative report, the noun is indicative of an element in the surgical footage, the particular inconsistency includes a characteristic of the element in the surgical footage being inconsistence with the adjective, and the suggested modification includes a replacement of the adjective with a different adjective selected based on an analysis of the surgical footage.

14. The non-transitory computer readable medium of claim 1, wherein the particular inconsistency includes an inconsistency in a duration of a phase of the surgical procedure, and the suggested modification includes a modification to the preliminary operative report to reflect the duration of the phase in the surgical procedure.

15. The non-transitory computer readable medium of claim 1, wherein the particular inconsistency includes an inconsistency in a surgical technique, and the suggested modification includes a modification to the preliminary operative report to indicate the surgical technique corresponding to the surgical footage.

16. The non-transitory computer readable medium of claim 1, wherein the preliminary operative report indicates at least one type of procedure, the particular inconsistency includes a particular type of procedure consistence with the surgical footage but not indicated by the preliminary operative report, and the suggested modification includes adding an indication of the particular type of procedure to the preliminary operative report.

17. The non-transitory computer readable medium of claim 1, wherein the particular inconsistency includes a particular type of procedure indicated by the preliminary operative report but inconsistence with the surgical footage, and the suggested modification includes removing the indication of the particular type of procedure from the preliminary operative report.

18. The non-transitory computer readable medium of claim 1, wherein the input received from the individual is an input in a natural language, and wherein the operations further comprise:
analyzing a plurality of frames of the surgical footage based on the input received from the individual to determine at least one phrase for insertion, the at least one phrase is not included in any one of the input received from the individual, the preliminary operative report and the suggested modification; and
inserting the at least one phrase into the preliminary operative report.

19. A system for assisted resolution of inconsistencies between surgical footages and operative reports, the system comprising:
at least one processing unit configured to perform operations, the operations comprise:
receiving an input of surgical footage of a surgical procedure performed on a patient by a health care provider;
receiving a preliminary operative report associated with the surgical procedure;
accessing a data-structure indicative of a plurality of inconsistencies between the surgical footage and the preliminary operative report;
for each inconsistency of the plurality of inconsistencies, presenting a respective visual indication of the inconsistency to an individual;
presenting the preliminary operative report to the individual;
receiving a selection of a particular inconsistency of the plurality of inconsistencies from the individual;
analyzing the preliminary operative report to generate a suggested modification to the preliminary operative report to correct the particular inconsistency;
presenting the suggested modification to the individual;
receiving an input from the individual indicative of a desire of the individual to alter the suggested modification;
updating the preliminary operative report based on the suggested modification and the input received from the individual; and
presenting the updated preliminary operative report to the individual.

20. A method for assisted resolution of inconsistencies between surgical footages and operative reports, the method comprising:
receiving an input of surgical footage of a surgical procedure performed on a patient by a health care provider;
receiving a preliminary operative report associated with the surgical procedure;
accessing a data-structure indicative of a plurality of inconsistencies between the surgical footage and the preliminary operative report;
for each inconsistency of the plurality of inconsistencies, presenting a respective visual indication of the inconsistency to an individual;
presenting the preliminary operative report to the individual;
receiving a selection of a particular inconsistency of the plurality of inconsistencies from the individual;
analyzing the preliminary operative report to generate a suggested modification to the preliminary operative report to correct the particular inconsistency;
presenting the suggested modification to the individual;
receiving an input from the individual indicative of a desire of the individual to alter the suggested modification;
updating the preliminary operative report based on the suggested modification and the input received from the individual; and
presenting the updated preliminary operative report to the individual.

21. A non-transitory computer readable medium containing instructions that when executed by at least one processor cause the at least one processor to perform operations, the operations comprising:
receiving an input of surgical footage of a surgical procedure performed on a patient by a health care provider;
receiving a preliminary operative report associated with the surgical procedure;
analyzing the preliminary operative report and the surgical footage to identify at least one inconsistency between the preliminary operative report and the surgical footage; and
providing an indication of the identified at least one inconsistency.

22. The non-transitory computer readable medium of claim 21, wherein the operations further comprise:
analyzing the preliminary operative report and the surgical footage to identify a portion of the preliminary operative report associated with the identified at least one inconsistency and a portion of the surgical footage associated with the identified at least one inconsistency; and
providing an indication of the identified portion of the preliminary operative report and an indication of the identified portion of the surgical footage.

23. The non-transitory computer readable medium of claim 21, wherein the operations further comprise:
accessing a textual medical history of the patient; and
analyzing the preliminary operative report, the surgical footage and the textual medical history of the patient to identify the at least one inconsistency between the preliminary operative report and the surgical footage.

24. The non-transitory computer readable medium of claim 21, wherein the operations further comprise:
accessing audio data recorded during the surgical procedure; and
analyzing the preliminary operative report, the surgical footage and the audio data to identify the at least one inconsistency between the preliminary operative report and the surgical footage.

25. The non-transitory computer readable medium of claim 21, wherein the provided indication of the identified at least one inconsistency includes a suggestion of a modification to the preliminary operative report to correct the at least one inconsistency, the modification includes at least one of (i) a replacement of a first adjective by a second adjective in the preliminary operative report, (ii) an automatically generated text and a position in the preliminary operative report for an insertion of the automatically generated text, or (iii) a removal of an automatically selected portion of the preliminary operative report.

26. The non-transitory computer readable medium of claim 21, wherein the operations further comprise:
identifying a first mathematical object in a mathematical space, the first mathematical object corresponds to at least part of the preliminary operative report;
calculating a convolution of at least part of the surgical footage to obtain a numerical value;
calculating a function of the first mathematical object and the numerical value to obtain a second mathematical object in the mathematical space; and
basing the identification of the at least one inconsistency between the preliminary operative report and the surgical footage on the second mathematical object.

27. The non-transitory computer readable medium of claim 21, wherein the identified at least one inconsistency includes an event depicted in the surgical footage that is not reflected in the preliminary operative report.

28. The non-transitory computer readable medium of claim 21, wherein the operations further comprise:
analyzing the preliminary operative report and the surgical footage to determine that a first event is depicted in the surgical footage and is not reflected in the preliminary operative report;
analyzing the preliminary operative report and the surgical footage to determine that a second event is depicted in the surgical footage and is not reflected in the preliminary operative report;
analyzing the surgical footage to determine a characteristic of the first event;
analyzing the surgical footage to determine a characteristic of the second event;
based on the characteristic of the first event, including the first event in the identified at least one inconsistency; and
based on the characteristic of the second event, excluding the second event from the identified at least one inconsistency.

29. The non-transitory computer readable medium of claim 21, wherein the identified at least one inconsistency includes at least one of a surgical tool depicted in the surgical footage that is not reflected in the preliminary operative report, an inconsistency in a quantity of surgical staples, or an inconsistency in a color associated with surgical staples.

30. The non-transitory computer readable medium of claim 21, wherein the operations further comprise:
analyzing the preliminary operative report and the surgical footage to determine that a first surgical tool is depicted in the surgical footage and is not reflected in the preliminary operative report;
analyzing the preliminary operative report and the surgical footage to determine that a second surgical tool is depicted in the surgical footage and is not reflected in the preliminary operative report;
analyzing the surgical footage to determine a characteristic of the first surgical tool;
analyzing the surgical footage to determine a characteristic of the second surgical tool;
based on the characteristic of the first surgical tool, including the first surgical tool in the identified at least one inconsistency; and
based on the characteristic of the second surgical tool, excluding the second surgical tool from the identified at least one inconsistency.

31. The non-transitory computer readable medium of claim 21, wherein the identified at least one inconsistency includes a secondary surgical trauma depicted in the surgical footage but not reflected in the preliminary operative report.

32. The non-transitory computer readable medium of claim 21, wherein the identified at least one inconsistency includes at least one of an anatomical structure depicted in the surgical footage in a condition that is not reflected in the preliminary operative report, or an anatomical structure depicted in the surgical footage in a size that is different from a size of the anatomical structure reflected in the preliminary operative report.

33. The non-transitory computer readable medium of claim 21, wherein the identified at least one inconsistency includes an event indicated in the preliminary operative report that is not depicted in the surgical footage.

34. The non-transitory computer readable medium of claim 21, wherein the identified at least one inconsistency includes a surgical tool indicated in the preliminary operative report that is not depicted in the surgical footage.

35. The non-transitory computer readable medium of claim 21, wherein the identified at least one inconsistency is based on a noun and an adjective adjacent to the noun in the preliminary operative report, and wherein the at least one inconsistency is based on a characteristic of an element in the surgical footage indicated by the noun being inconsistence with the adjective.

36. The non-transitory computer readable medium of claim 21, wherein the identified at least one inconsistency is based on an order of events in the surgical footage being different from an order of the events indicated in the preliminary operative report.

37. The non-transitory computer readable medium of claim 21, wherein the identified at least one inconsistency includes an inconsistency in a duration of a phase of the surgical procedure.

38. The non-transitory computer readable medium of claim 21, wherein the identified at least one inconsistency includes at least one of an inconsistency in a surgical technique or an inconsistency a type of procedure consistence with the surgical footage that is not indicated by the preliminary operative report.

39. A system, the system comprising:
at least one processing unit configured to perform operations, the operations comprise:
receiving an input of surgical footage of a surgical procedure performed on a patient by a health care provider;
receiving a preliminary operative report associated with the surgical procedure;
analyzing the preliminary operative report and the surgical footage to identify at least one inconsistency between the preliminary operative report and the surgical footage; and
providing an indication of the identified at least one inconsistency.

40. A method, the method comprising:
receiving an input of surgical footage of a surgical procedure performed on a patient by a health care provider;
receiving a preliminary operative report associated with the surgical procedure;
analyzing the preliminary operative report and the surgical footage to identify at least one inconsistency between the preliminary operative report and the surgical footage; and
providing an indication of the identified at least one inconsistency.

41. A non-transitory computer readable medium containing instructions that when executed by at least one processor cause the at least one processor to perform operations, the operations comprising:
receiving an input of an identifier of a patient;
receiving an input of an identifier of a health care provider;
receiving an input of surgical footage of a surgical procedure performed on the patient by the health care provider;
analyzing a plurality of frames of the surgical footage to derive image-based text for populating an operative report of the surgical procedure; and
causing the derived image-based text to populate the operative report of the surgical procedure.

42. The non-transitory computer readable medium of claim 41, wherein the derived image-based text includes a noun indicative of an anatomical structure depicted in the surgical footage, and an adjective adjacent to the noun, the adjective is indicative of a characteristic of the anatomical structure.

43. The non-transitory computer readable medium of claim 41, wherein the derived image-based text includes a noun indicative of an event depicted in the surgical footage, and an adjective adjacent to the noun, the adjective is indicative of a characteristic of the event.

44. The non-transitory computer readable medium of claim 41, wherein the derived image-based text includes a noun indicative of a surgical tool depicted in the surgical footage, and an adjective adjacent to the noun, the adjective is indicative of a characteristic of the surgical tool.

45. The non-transitory computer readable medium of claim 41, wherein the derived image-based text includes a verb indicative of a surgical action depicted in the surgical footage, and an adverb adjacent to the verb, the adverb is indicative of a characteristic of the surgical action.

46. The non-transitory computer readable medium of claim 41, wherein the derived image-based text is derived in a style selected based on the health care provider.

47. The non-transitory computer readable medium of claim 41, wherein the derived image-based text is indicative of at least one of a surgical technique depicted in the surgical footage or of a plurality of types of the surgical procedure depicted in the surgical footage.

48. The non-transitory computer readable medium of claim 41, wherein the derived image-based text is indicative of a duration of a phase of the surgical procedure.

49. The non-transitory computer readable medium of claim 41, wherein the operations further comprise:
analyzing the plurality of frames to identify a first surgical event, a second surgical event, and a third surgical event;
analyzing the plurality of frames to determine a temporal order of the first, second and third surgical events; and
including in the derived image-based text an indication of the temporal order of the first, second and third surgical events.

50. The non-transitory computer readable medium of claim 41, wherein operations further comprise:
approaching the health care provider via a user interface to obtain a specific detail, the specific detail is not available from the surgical footage; and
including information based on the specific detail in the derived image-based text.

51. The non-transitory computer readable medium of claim 41, wherein the operations further comprise:
accessing a textual medical history of the patient; and
analyzing the plurality of frames and the textual medical history of the patient to identify information from the textual medical history for integration into the operative report.

52. The non-transitory computer readable medium of claim 41, wherein the operations further comprise:
accessing audio data recorded during the surgical procedure; and
analyzing the plurality of frames and the audio data to determine information based on the audio data for integration into the operative report.

53. The non-transitory computer readable medium of claim 41, wherein the derived image-based text includes an indication that a specific surgical action was not performed during the surgical procedure.

54. The non-transitory computer readable medium of claim 41, wherein the derived image-based text includes a post-operative diagnosis, and the operations further comprise analyzing the plurality of frames to determine the post-operative diagnosis.

55. The non-transitory computer readable medium of claim 41, wherein the derived image-based text includes an indication of at least one intraoperative complication determined based on an analysis of the plurality of frames.

56. The non-transitory computer readable medium of claim 41, wherein the derived image-based text includes a first portion and a second portion, the first portion includes summarized key details observed during the surgical procedure, and the second portion includes detailed description of the surgical procedure.

57. The non-transitory computer readable medium of claim 41, wherein the operations further comprise:
analyzing the plurality of frames of the surgical footage to identify a first intraoperative event and a second intraoperative event;
determining a first level of implication associated with the first intraoperative event;
determining a second level of implication associated with the second intraoperative event;
based on the first level of implication, excluding any indication of the first intraoperative event from the derived image-based text; and
based on the second level of implication, including an indication of the second intraoperative event in the derived image-based text.

58. The non-transitory computer readable medium of claim 41, wherein the operations further comprise:
calculating a convolution of a first part of the surgical footage to obtain a first numerical result value;
calculating a convolution of a second part of the surgical footage to obtain a second numerical result value;
calculating a convolution of a third part of the surgical footage to obtain a third numerical result value;
calculating a function of the first numerical result value and the second numerical result value to obtain a first mathematical object;
calculating a function of the first numerical result value and the third numerical result value to obtain a second mathematical object;
selecting an adjective based on the first mathematical object;
selecting a noun based on the second mathematical object; and
including the adjective and the noun in the derived image-based text.

59. A system, the system comprising:
at least one processing unit configured to perform operations, the operations comprise:
receiving an input of an identifier of a patient;
receiving an input of an identifier of a health care provider;
receiving an input of surgical footage of a surgical procedure performed on the patient by the health care provider;
analyzing a plurality of frames of the surgical footage to derive image-based text for populating an operative report of the surgical procedure; and
causing the derived image-based text to populate the operative report of the surgical procedure.

60. A method, the method comprising:
receiving an input of an identifier of a patient;
receiving an input of an identifier of a health care provider;
receiving an input of surgical footage of a surgical procedure performed on the patient by the health care provider;
analyzing a plurality of frames of the surgical footage to derive image-based text for populating an operative report of the surgical procedure; and
causing the derived image-based text to populate the operative report of the surgical procedure.

61. A non-transitory computer readable medium containing instructions that when executed by at least one processor cause the at least one processor to perform operations for synchronized browsing of surgical videos and operative reports, the operations comprising:
receiving an input of surgical footage of a surgical procedure performed on a patient by a health care provider;
receiving textual data in a natural language, the textual data is associated with the surgical procedure;
accessing a data-structure associating different portions of the surgical footage with different portions of the textual data;
presenting the textual data and a first part of the surgical footage to an individual, with a visual indication of a first part of the textual data, the first part of the surgical footage is associated in the data-structure with the first part of the textual data;
receiving from the individual a selection of a second part of the textual data;
using the data-structure to determine a second part of the surgical footage associated with the second part of the textual data;
presenting the second part of the surgical footage to the individual;
receiving from the individual a selection of a third part of the surgical footage;
using the data-structure to determine a third part of the textual data associated with the third part of the surgical footage; and
presenting a visual indication of the third part of the textual data to the individual.

62. The non-transitory computer readable medium of claim 61, wherein the textual data is textual data from an operative report associated with the surgical procedure.

63. The non-transitory computer readable medium of claim 61, wherein each of the first, second and third parts of the textual data includes a respective first textual portion in the natural language automatically generated based on a respective analysis of a respective plurality of frames and a respective second textual portion in the natural language manually entered by the health care provider.

64. The non-transitory computer readable medium of claim 61, wherein the operations further comprise analyzing a plurality of frames of the surgical footage and the textual data to generate the data-structure.

65. The non-transitory computer readable medium of claim 64, wherein the analyzing includes:
identifying a first mathematical object in a mathematical space, the first mathematical object corresponds to at least part of a first portion of the textual data;
identifying a second mathematical object in a mathematical space, the second mathematical object corresponds to at least part of a second portion of the textual data;
calculating a convolution of at least part of a first portion of the surgical footage to obtain a first numerical value;
calculating a convolution of at least part of a second portion of the surgical footage to obtain a second numerical value;
calculating a function of the first mathematical object and the first numerical value to obtain a third mathematical object in the mathematical space;
based on the third mathematical object, associating in the data-structure the first portion of the surgical footage with the first portion of the textual data;
calculating a function of the first mathematical object and the second numerical value to obtain a fourth mathematical object in the mathematical space;
based on the fourth mathematical object, avoiding associating in the data-structure the second portion of the surgical footage with the first portion of the textual data;
calculating a function of the second mathematical object and the second numerical value to obtain a fifth mathematical object in the mathematical space; and
based on the fifth mathematical object, associating in the data-structure the second portion of the surgical footage with the second portion of the textual data.

66. The non-transitory computer readable medium of claim 61, wherein the operations further comprise:
receiving from the individual a modification to the textual data; and
analyzing a plurality of frames of the surgical footage and the modified textual data to update at least one association in the data-structure.

67. The non-transitory computer readable medium of claim 61, wherein a portion of the surgical footage depicting a surgical event is associated in the data-structure with a portion of the textual data indicative of the surgical event.

68. The non-transitory computer readable medium of claim 61, wherein a portion of the surgical footage depicting a surgical tool is associated in the data-structure with a portion of the textual data indicative of an alternative surgical tool.

69. The non-transitory computer readable medium of claim 61, wherein a portion of the surgical footage is associated in the data-structure with a portion of the textual data inconsistence with the portion of the surgical footage.

70. The non-transitory computer readable medium of claim 69, wherein the operations further comprise presenting the portion of the surgical footage to the individual, with a visual indication of the portion of the textual data, and a suggested modification to the portion of the textual data to correct the inconsistency.

71. The non-transitory computer readable medium of claim 61, wherein a first portion of the surgical footage depicts a first object, a second portion of the surgical footage depicts a second object, a specific noun is indicative of both the first and second objects, a first portion of the textual data includes the specific noun adjacent to a first adjective, a second portion of the textual data includes the specific noun adjacent to a second adjective, and the data-structure associates the first portion of the surgical footage with the first portion of the textual data based on the first adjective and a characteristic of the first object in the surgical footage, and associates the second portion of the surgical footage with the second portion of the textual data based on the second adjective and a characteristic of the second object in the surgical footage.

72. The non-transitory computer readable medium of claim 61, wherein a first portion of the surgical footage depicts a first event, a second portion of the surgical footage depicts a second event, a specific verb is indicative of both the first and second events, a first portion of the textual data includes the specific verb adjacent to a first adverb, a second portion of the textual data includes the specific verb adjacent to a second adverb, and the data-structure associates the first portion of the surgical footage with the first portion of the textual data based on the first adverb and a characteristic of the first event in the surgical footage, and associates the second portion of the surgical footage with the second portion of the textual data based on the second adverb and a characteristic of the second event in the surgical footage.

73. The non-transitory computer readable medium of claim 61, wherein the operations further comprise:
receiving from the individual a selection of a fourth part of the surgical footage;
determining that the fourth part of the surgical footage is not associated with any portion of the textual data in the data-structure; and
providing a visual indication of the lack of association.

74. The non-transitory computer readable medium of claim 61, wherein the operations further comprise:
receiving from the individual a selection of a fourth part of the textual data;
determining that the fourth part of the textual data is not associated with any portion of the surgical footage in the data-structure; and
providing a visual indication of the lack of association.

75. The non-transitory computer readable medium of claim 61, wherein the operations further comprise:
presenting a plurality of issues associated with the textual data to the individual;
receiving from the individual a selection of a particular issue of the plurality of issues; and
in response to the selection of the particular issue, presenting to the individual a portion of the surgical footage associated with the particular issue and a visual indication of a portion of the textual data associated with the particular issue.

76. The non-transitory computer readable medium of claim 61, wherein the operations further comprise:
while presenting the first part of the surgical footage and the visual indication of the first part of the textual data, receiving an input from the individual for correcting the association in the data-structure of the first part of the surgical footage with the first part of the textual data; and
updating the data-structure based on the input from the individual.

77. The non-transitory computer readable medium of claim 61, wherein the operations further comprise providing a user interface control to enable the individual to select a mode of presentation from at least a first mode of presentation and a second mode of presentation, wherein in the first mode of presentation, a selection of a segment of the textual data automatically causes a presentation of a segment of the surgical footage associated with the selected segment of the textual data, and wherein in the second mode of presentation, the selection of the segment of the textual data does not cause an automatic presentation of the segment of the surgical footage associated with the selected segment of the textual data.

78. The non-transitory computer readable medium of claim 61, wherein the operations further comprise:
presenting a playback of the surgical footage;
while the playback is in a first portion of the surgical footage, presenting a visual indication of a first portion of the textual data, the first portion of the surgical footage is associated in the data-structure with the first portion of the textual data; and
while the playback is in a second portion of the surgical footage, presenting a visual indication of a second portion of the textual data, the second portion of the surgical footage is associated in the data-structure with the second portion of the textual data.

79. A system for synchronized browsing of surgical videos and operative reports, the system comprising:
at least one processing unit configured to perform operations, the operations comprise:
receiving an input of surgical footage of a surgical procedure performed on a patient by a health care provider;
receiving textual data in a natural language, the textual data is associated with the surgical procedure;
accessing a data-structure associating different portions of the surgical footage with different portions of the textual data;
presenting the textual data and a first part of the surgical footage to an individual, with a visual indication of a first part of the textual data, the first part of the surgical footage is associated in the data-structure with the first part of the textual data;
receiving from the individual a selection of a second part of the textual data;
using the data-structure to determine a second part of the surgical footage associated with the second part of the textual data;
presenting the second part of the surgical footage to the individual;
receiving from the individual a selection of a third part of the surgical footage;
using the data-structure to determine a third part of the textual data associated with the third part of the surgical footage; and
presenting a visual indication of the third part of the textual data to the individual.

80. A method for synchronized browsing of surgical videos and operative reports, the method comprising:
receiving an input of surgical footage of a surgical procedure performed on a patient by a health care provider;
receiving textual data in a natural language, the textual data is associated with the surgical procedure;
accessing a data-structure associating different portions of the surgical footage with different portions of the textual data;
presenting the textual data and a first part of the surgical footage to an individual, with a visual indication of a first part of the textual data, the first part of the surgical footage is associated in the data-structure with the first part of the textual data;
receiving from the individual a selection of a second part of the textual data;
using the data-structure to determine a second part of the surgical footage associated with the second part of the textual data;
presenting the second part of the surgical footage to the individual;
receiving from the individual a selection of a third part of the surgical footage;
using the data-structure to determine a third part of the textual data associated with the third part of the surgical footage; and
presenting a visual indication of the third part of the textual data to the individual.
